SEQUENCE LISTING (59 sequences)

SEQ ID NO: 1 - protein, 118 aa, synthetic construct
  source 1..118: note = Chimeric VH AA
EVQLQQSGAELVKPGASVKLSCTASGFNIKDTYMHWVKQRPEQGLEWIGRIDPANGNTQYDPKFQGKATIPADTSSNTAYLQLSSLTSEDTAVYYCVYGRGSAWFAFWGQGTLVTVSA

SEQ ID NO: 2 - protein, 107 aa, synthetic construct
  source 1..107: note = Chimeric VL AA
DIQMTQTTSSLSASLGDRVTISCRASQDISNYLNWYQQKPDGTVKLLIYYTSRLHSGVPSRFSGSGSGTDYSLTISNLEQEDIATYFCQQGNTLPWTFGGGTKLEIK

SEQ ID NO: 3 - protein, 8 aa, synthetic construct
  source 1..8: note = CDR H1 AA
GFNIKDTY

SEQ ID NO: 4 - protein, 8 aa, synthetic construct
  source 1..8: note = CDR H2 AA
IDPANGNT

SEQ ID NO: 5 - protein, 11 aa, synthetic construct
  source 1..11: note = CDR H3 AA
VYGRGSAWFAF

SEQ ID NO: 6 - protein, 6 aa, synthetic construct
  source 1..6: note = CDR L1 AA
QDISNY

SEQ ID NO: 7 - skipped

SEQ ID NO: 8 - protein, 9 aa, synthetic construct
  source 1..9: note = CDR L3 AA
QQGNTLPWT

SEQ ID NO: 9 - protein, 484 aa, synthetic construct
  source 1..484: note = Chimeric Antigen Receptor AA #1 (4-1BB)
MALPVTALLLPLALLLHAARPDIQMTQTTSSLSASLGDRVTISCRASQDISNYLNWYQQKPDGTVKLLIYYTSRLHSGVPSRFSGSGSGTDYSLTISNLEQEDIATYFCQQGNTLPWTFGGGTKLEIKGGGGSGGGGSGGGGSEVQLQQSGAELVKPGASVKLSCTASGFNIKDTYMHWVKQRPEQGLEWIGRIDPANGNTQYDPKFQGKATIPADTSSNTAYLQLSSLTSEDTAVYYCVYGRGSAWFAFWGQGTLVTVSATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 10 - DNA, 354 bp, synthetic construct
  source 1..354: mol_type = other DNA; note = VH DNA (CAR)
gaggtgcagctgcaacaaagcggcgccgagctggtgaagcccggcgccagcgtgaaactgagctgtaccgctagcggatttaacatcaaggacacatatatgcactgggtcaaacagcggcccgagcagggcctcgaatggatcggccggatcgaccctgccaacggcaatactcaatacgaccccaagttccagggcaaggccacaattcctgctgatacatcttctaataccgcctatctgcagctgagcagcctgacctccgaggataccgccgtgtactactgcgtgtacggcagaggctctgcctggttcgccttttggggccagggaaccctggttacggtgtccgcc

SEQ ID NO: 11 - DNA, 321 bp, synthetic construct
  source 1..321: mol_type = other DNA; note = VL DNA (CAR)
gacatccagatgacacagaccaccagcagcctgtcagcttctctgggagatagagtgaccatcagctgcagagccagccaggacatcagcaactacctgaactggtatcagcagaagcctgatggcacagtgaagctgctgatctactacaccagccggctgcacagcggcgtgccatctagattcagcggaagcggcagcggcaccgactacagcctcacaatcagcaacctggaacaggaggacatcgccacctacttctgccagcagggcaacacactgccttggaccttcggcggcggcaccaagctggaaatcaag

SEQ ID NO: 12 - DNA, 24 bp, synthetic construct
  source 1..24: mol_type = other DNA; note = CDR H1 DNA
ggatttaacatcaaggacacatat

SEQ ID NO: 13 - DNA, 24 bp, synthetic construct
  source 1..24: mol_type = other DNA; note = CDR H2 DNA
atcgaccctgccaacggcaatact

SEQ ID NO: 14 - DNA, 45 bp, synthetic construct
  source 1..45: mol_type = other DNA; note = CDR H3 DNA
gtgtactactgcgtgtacggcagaggctctgcctggttcgccttt

SEQ ID NO: 15 - DNA, 18 bp, synthetic construct
  source 1..18: mol_type = other DNA; note = CDR L1 DNA
caggacatcagcaactac

SEQ ID NO: 16 - skipped

SEQ ID NO: 17 - DNA, 27 bp, synthetic construct
  source 1..27: mol_type = other DNA; note = CDR L3 DNA
cagcagggcaacacactgccttggacc

SEQ ID NO: 18 - DNA, 1455 bp, synthetic construct
  source 1..1455: mol_type = other DNA; note = Chimeric Antigen Receptor DNA #1 (4-1BB)
atggccctgcctgtgaccgccctgctgctgccactggccctgctgctgcacgccgctagacctgacatccagatgacacagaccaccagcagcctgtcagcttctctgggagatagagtgaccatcagctgcagagccagccaggacatcagcaactacctgaactggtatcagcagaagcctgatggcacagtgaagctgctgatctactacaccagccggctgcacagcggcgtgccatctagattcagcggaagcggcagcggcaccgactacagcctcacaatcagcaacctggaacaggaggacatcgccacctacttctgccagcagggcaacacactgccttggaccttcggcggcggcaccaagctggaaatcaagggcggaggcggcagcggcggcggaggctctggcggcggaggttccgaggtgcagctgcaacaaagcggcgccgagctggtgaagcccggcgccagcgtgaaactgagctgtaccgctagcggatttaacatcaaggacacatatatgcactgggtcaaacagcggcccgagcagggcctcgaatggatcggccggatcgaccctgccaacggcaatactcaatacgaccccaagttccagggcaaggccacaattcctgctgatacatcttctaataccgcctatctgcagctgagcagcctgacctccgaggataccgccgtgtactactgcgtgtacggcagaggctctgcctggttcgccttttggggccagggaaccctggttacggtgtccgccacaaccacccctgcccctagacctcctacccctgctccaaccatcgccagccagcccctgtctctgcggcctgaagcttgcagacccgctgctggcggcgccgtgcacacgaggggactggacttcgcctgcgacatctacatttgggcccctctggccggcacctgcggcgtgctgctgctgagcctggtcatcacactgtactgcaagagaggcagaaaaaagctgctgtacatcttcaagcagccttttatgcggcccgtgcagaccacacaggaggaagatggctgctcttgtagattccccgaggaagaggaaggcggatgtgaactgagagtgaagttcagcagaagcgccgatgcccctgcctaccagcagggacagaaccagctgtacaacgagctgaacctgggcagacgggaagagtacgacgtgctggataagcggcggggtcgggaccccgagatgggaggcaagcctagaaggaagaacccccaagagggcctttataatgagctgcagaaagataagatggccgaagcctacagcgagatcggcatgaagggcgagagacgcagaggcaaaggccacgacggcctgtaccaaggcctctccaccgccacaaaggacacctacgacgccctgcacatgcaggccctgcctccaagatga

SEQ ID NO: 19 - protein, 5 aa, synthetic construct
  source 1..5: note = CDR H1 AA
DTYMH

SEQ ID NO: 20 - protein, 17 aa, synthetic construct
  source 1..17: note = CDR H2 AA
RIDPANGNTQYDPKFQG

SEQ ID NO: 21 - protein, 9 aa, synthetic construct
  source 1..9: note = CDR H3 AA
GRGSAWFAF

SEQ ID NO: 22 - protein, 11 aa, synthetic construct
  source 1..11: note = CDR L1 AA
RASQDISNYLN

SEQ ID NO: 23 - protein, 7 aa, synthetic construct
  source 1..7: note = CDR L2 AA
YTSRLHS

SEQ ID NO: 24 - protein, 6 aa, synthetic construct
  source 1..6: note = CDR L2 AA
YTSRLH

SEQ ID NO: 25 - DNA, 15 bp, synthetic construct
  source 1..15: mol_type = other DNA; note = CDR H1 DNA
gacacatatatgcac

SEQ ID NO: 26 - DNA, 51 bp, synthetic construct
  source 1..51: mol_type = other DNA; note = CDR H2 DNA
cggatcgaccctgccaacggcaatactcaatacgaccccaagttccagggc

SEQ ID NO: 27 - DNA, 36 bp, synthetic construct
  source 1..36: mol_type = other DNA; note = CDR H3 DNA
tgcgtgtacggcagaggctctgcctggttcgccttt

SEQ ID NO: 28 - DNA, 33 bp, synthetic construct
  source 1..33: mol_type = other DNA; note = CDR L1 DNA
agagccagccaggacatcagcaactacctgaac

SEQ ID NO: 29 - DNA, 21 bp, synthetic construct
  source 1..21: mol_type = other DNA; note = CDR L2 DNA
tacaccagccggctgcacagc

SEQ ID NO: 30 - DNA, 18 bp, synthetic construct
  source 1..18: mol_type = other DNA; note = CDR L2 DNA
tacaccagccggctgcac

SEQ ID NO: 31 - protein, 179 aa, synthetic construct
  source 1..179: note = human CD94
MAVFKTTLWRLISGTLGIICLSLMSTLGILLKNSFTKLSIEPAFTPGPNIELQKDSDCCSCQEKWVGYRCNCYFISSEQKTWNESRHLCASQKSSLLQLQNTDELDFMSSSQQFYWIGLSYSEEHTAWLWENGSALSQYLFPSFETFNTKNCIAYNPNGNALDESCEDKNRYICKQQLI

SEQ ID NO: 32 - protein, 21 aa, synthetic construct
  source 1..21: note = CD8 alpha signal peptide AA
MALPVTALLLPLALLLHAARP

SEQ ID NO: 33 - protein, 240 aa, synthetic construct
  source 1..240: note = scFv AA
DIQMTQTTSSLSASLGDRVTISCRASQDISNYLNWYQQKPDGTVKLLIYYTSRLHSGVPSRFSGSGSGTDYSLTISNLEQEDIATYFCQQGNTLPWTFGGGTKLEIKGGGGSGGGGSGGGGSEVQLQQSGAELVKPGASVKLSCTASGFNIKDTYMHWVKQRPEQGLEWIGRIDPANGNTQYDPKFQGKATIPADTSSNTAYLQLSSLTSEDTAVYYCVYGRGSAWFAFWGQGTLVTVSA

SEQ ID NO: 34 - protein, 15 aa, synthetic construct
  source 1..15: note = linker AA
GGGGSGGGGSGGGGS

SEQ ID NO: 35 - protein, 45 aa, synthetic construct
  source 1..45: note = CD8 alpha hinge domain AA
TTTPAPRPPTPAPTIASQPLSLRPEACRPAAGGAVHTRGLDFACD

SEQ ID NO: 36 - protein, 24 aa, synthetic construct
  source 1..24: note = CD8 alpha transmembrane domain AA
IYIWAPLAGTCGVLLLSLVITLYC

SEQ ID NO: 37 - protein, 42 aa, synthetic construct
  source 1..42: note = 4-1BB costimulatory domain AA
KRGRKKLLYIFKQPFMRPVQTTQEEDGCSCRFPEEEEGGCEL

SEQ ID NO: 38 - protein, 112 aa, synthetic construct
  source 1..112: note = CD3 zeta costimulatory domain AA
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 39 - DNA, 63 bp, synthetic construct
  source 1..63: mol_type = other DNA; note = CD8 alpha signal peptide DNA
atggccctgcctgtgaccgccctgctgctgccactggccctgctgctgcacgccgctagacct

SEQ ID NO: 40 - DNA, 720 bp, synthetic construct
  source 1..720: mol_type = other DNA; note = scFv DNA
gacatccagatgacacagaccaccagcagcctgtcagcttctctgggagatagagtgaccatcagctgcagagccagccaggacatcagcaactacctgaactggtatcagcagaagcctgatggcacagtgaagctgctgatctactacaccagccggctgcacagcggcgtgccatctagattcagcggaagcggcagcggcaccgactacagcctcacaatcagcaacctggaacaggaggacatcgccacctacttctgccagcagggcaacacactgccttggaccttcggcggcggcaccaagctggaaatcaagggcggaggcggcagcggcggcggaggctctggcggcggaggttccgaggtgcagctgcaacaaagcggcgccgagctggtgaagcccggcgccagcgtgaaactgagctgtaccgctagcggatttaacatcaaggacacatatatgcactgggtcaaacagcggcccgagcagggcctcgaatggatcggccggatcgaccctgccaacggcaatactcaatacgaccccaagttccagggcaaggccacaattcctgctgatacatcttctaataccgcctatctgcagctgagcagcctgacctccgaggataccgccgtgtactactgcgtgtacggcagaggctctgcctggttcgccttttggggccagggaaccctggttacggtgtccgcc

SEQ ID NO: 41 - DNA, 45 bp, synthetic construct
  source 1..45: mol_type = other DNA; note = linker DNA
ggcggaggcggcagcggcggcggaggctctggcggcggaggttcc

SEQ ID NO: 42 - DNA, 135 bp, synthetic construct
  source 1..135: mol_type = other DNA; note = CD8 alpha hinge domain DNA
acaaccacccctgcccctagacctcctacccctgctccaaccatcgccagccagcccctgtctctgcggcctgaagcttgcagacccgctgctggcggcgccgtgcacacgaggggactggacttcgcctgcgac

SEQ ID NO: 43 - DNA, 72 bp, synthetic construct
  source 1..72: mol_type = other DNA; note = CD8 alpha transmembrane domain DNA
atctacatttgggcccctctggccggcacctgcggcgtgctgctgctgagcctggtcatcacactgtactgc

SEQ ID NO: 44 - DNA, 126 bp, synthetic construct
  source 1..126: mol_type = other DNA; note = 4-1BB costimulatory domain DNA
aagagaggcagaaaaaagctgctgtacatcttcaagcagccttttatgcggcccgtgcagaccacacaggaggaagatggctgctcttgtagattccccgaggaagaggaaggcggatgtgaactg

SEQ ID NO: 45 - DNA, 339 bp, synthetic construct
  source 1..339: mol_type = other DNA; note = CD3 zeta costimulatory domain DNA
agagtgaagttcagcagaagcgccgatgcccctgcctaccagcagggacagaaccagctgtacaacgagctgaacctgggcagacgggaagagtacgacgtgctggataagcggcggggtcgggaccccgagatgggaggcaagcctagaaggaagaacccccaagagggcctttataatgagctgcagaaagataagatggccgaagcctacagcgagatcggcatgaagggcgagagacgcagaggcaaaggccacgacggcctgtaccaaggcctctccaccgccacaaaggacacctacgacgccctgcacatgcaggccctgcctccaagatga

SEQ ID NO: 46 - protein, 448 aa, synthetic construct
  source 1..448: note = Chimeric Heavy chain AA
EVQLQQSGAELVKPGASVKLSCTASGFNIKDTYMHWVKQRPEQGLEWIGRIDPANGNTQYDPKFQGKATIPADTSSNTAYLQLSSLTSEDTAVYYCVYGRGSAWFAFWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 47 - protein, 214 aa, synthetic construct
  source 1..214: note = Chimeric Light chain AA
DIQMTQTTSSLSASLGDRVTISCRASQDISNYLNWYQQKPDGTVKLLIYYTSRLHSGVPSRFSGSGSGTDYSLTISNLEQEDIATYFCQQGNTLPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 48 - DNA, 1347 bp, synthetic construct
  source 1..1347: mol_type = other DNA; note = Chimeric Heavy chain DNA
gaggttcagctgcaacagtctggggcagagcttgtgaagccaggggcctcagtcaagttgtcctgcacagcttctggcttcaacattaaagacacctatatgcactgggtgaagcagaggcctgaacagggcctggagtggattggaaggattgatcctgcgaatggtaatactcaatatgacccgaagttccagggcaaggccactataccagcagacacatcctccaacacagcctacctgcagctcagcagcctgacatctgaggacactgccgtctattattgtgtctacggtcgtggctcggcctggtttgctttctggggccaagggactctggtcactgtctctgcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagccgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggacgagctcaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 49 - DNA, 517 bp, synthetic construct
  source 1..517: mol_type = other DNA; note = Chimeric Light chain DNA
gatatccagatgacacagactacatcctccctgtctgcctctctgggagacagagtcaccatcagttgcagggcaagtcaggacattagcaattatttaaactggtatcagcagaaaccagatggaactgttaaactcctgatctactacacatcaagattacactcaggagtcccatcaaggttcagtggcagtgggtctggaacagattattctctcaccattagcaacctggagcaagaagatattgccacttacttttgtcaacagggtaatacgcttccgtggacgttcggtggaggcaccaagctggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacct

SEQ ID NO: 50 - DNA, 354 bp, synthetic construct
  source 1..354: mol_type = other DNA; note = Chimeric VH DNA
gaggttcagctgcaacagtctggggcagagcttgtgaagccaggggcctcagtcaagttgtcctgcacagcttctggcttcaacattaaagacacctatatgcactgggtgaagcagaggcctgaacagggcctggagtggattggaaggattgatcctgcgaatggtaatactcaatatgacccgaagttccagggcaaggccactataccagcagacacatcctccaacacagcctacctgcagctcagcagcctgacatctgaggacactgccgtctattattgtgtctacggtcgtggctcggcctggtttgctttctggggccaagggactctggtcactgtctctgca

SEQ ID NO: 51 - DNA, 324 bp, synthetic construct
  source 1..324: mol_type = other DNA; note = Chimeric VL DNA
gatatccagatgacacagactacatcctccctgtctgcctctctgggagacagagtcaccatcagttgcagggcaagtcaggacattagcaattatttaaactggtatcagcagaaaccagatggaactgttaaactcctgatctactacacatcaagattacactcaggagtcccatcaaggttcagtggcagtgggtctggaacagattattctctcaccattagcaacctggagcaagaagatattgccacttacttttgtcaacagggtaatacgcttccgtggacgttcggtggaggcaccaagctggaaatcaaacgt

SEQ ID NO: 52 - protein, 37 aa, Synthetic construct
  source 1..37: note = CD28 costimulatory domain AA
KRSRLLHSDYMNMTPRRPGPTRKHYQPYAPPRDFAAY

SEQ ID NO: 53 - protein, 37 aa, Synthetic construct
  source 1..37: note = OX40 costimulatory domain AA
RRDQRLPPDAHKPPGGGSFRTPIQEEQADAHSTLAKI

SEQ ID NO: 54 - DNA, 111 bp, Synthetic construct
  source 1..111: mol_type = other DNA; note = CD28 costimulatory domain DNA
aaacgcagccgcctgctgcatagcgattatatgaacatgaccccgcgccgcccgggcccgacccgcaaacattatcagccgtatgcgccgccgcgcgattttgcggcgtat

SEQ ID NO: 55 - DNA, 111 bp, Synthetic construct
  source 1..111: mol_type = other DNA; note = OX40 costimulatory domain DNA
cggagggaccagaggctgccccccgatgcccacaagccccctgggggaggcagtttccggacccccatccaagaggagcaggccgacgcccactccaccctggccaagatc

SEQ ID NO: 56 - protein, 479 aa, Synthetic construct
  source 1..479: note = Chimeric Antigen Receptor AA #2 (CD28)
MALPVTALLLPLALLLHAARPDIQMTQTTSSLSASLGDRVTISCRASQDISNYLNWYQQKPDGTVKLLIYYTSRLHSGVPSRFSGSGSGTDYSLTISNLEQEDIATYFCQQGNTLPWTFGGGTKLEIKGGGGSGGGGSGGGGSEVQLQQSGAELVKPGASVKLSCTASGFNIKDTYMHWVKQRPEQGLEWIGRIDPANGNTQYDPKFQGKATIPADTSSNTAYLQLSSLTSEDTAVYYCVYGRGSAWFAFWGQGTLVTVSATTTPAPRPPTPAPTIASQPLSLRPEACRPAAGGAVHTRGLDFACDIYIWAPLAGTCGVLLLSLVITLYCKRSRLLHSDYMNMTPRRPGPTRKHYQPYAPPRDFAAYRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 57 - protein, 479 aa, Synthetic construct
  source 1..479: note = Chimeric Antigen Receptor AA #3 (OX40)
MALPVTALLLPLALLLHAARPDIQMTQTTSSLSASLGDRVTISCRASQDISNYLNWYQQKPDGTVKLLIYYTSRLHSGVPSRFSGSGSGTDYSLTISNLEQEDIATYFCQQGNTLPWTFGGGTKLEIKGGGGSGGGGSGGGGSEVQLQQSGAELVKPGASVKLSCTASGFNIKDTYMHWVKQRPEQGLEWIGRIDPANGNTQYDPKFQGKATIPADTSSNTAYLQLSSLTSEDTAVYYCVYGRGSAWFAFWGQGTLVTVSATTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 58 - DNA, 1440 bp, Synthetic construct
  source 1..1440: mol_type = other DNA; note = Chimeric Antigen Receptor DNA #2 (CD28)
atggccctgcctgtgaccgccctgctgctgccactggccctgctgctgcacgccgctagacctgacatccagatgacacagaccaccagcagcctgtcagcttctctgggagatagagtgaccatcagctgcagagccagccaggacatcagcaactacctgaactggtatcagcagaagcctgatggcacagtgaagctgctgatctactacaccagccggctgcacagcggcgtgccatctagattcagcggaagcggcagcggcaccgactacagcctcacaatcagcaacctggaacaggaggacatcgccacctacttctgccagcagggcaacacactgccttggaccttcggcggcggcaccaagctggaaatcaagggcggaggcggcagcggcggcggaggctctggcggcggaggttccgaggtgcagctgcaacaaagcggcgccgagctggtgaagcccggcgccagcgtgaaactgagctgtaccgctagcggatttaacatcaaggacacatatatgcactgggtcaaacagcggcccgagcagggcctcgaatggatcggccggatcgaccctgccaacggcaatactcaatacgaccccaagttccagggcaaggccacaattcctgctgatacatcttctaataccgcctatctgcagctgagcagcctgacctccgaggataccgccgtgtactactgcgtgtacggcagaggctctgcctggttcgccttttggggccagggaaccctggttacggtgtccgccacaaccacccctgcccctagacctcctacccctgctccaaccatcgccagccagcccctgtctctgcggcctgaagcttgcagacccgctgctggcggcgccgtgcacacgaggggactggacttcgcctgcgacatctacatttgggcccctctggccggcacctgcggcgtgctgctgctgagcctggtcatcacactgtactgcaaacgcagccgcctgctgcatagcgattatatgaacatgaccccgcgccgcccgggcccgacccgcaaacattatcagccgtatgcgccgccgcgcgattttgcggcgtatagagtgaagttcagcagaagcgccgatgcccctgcctaccagcagggacagaaccagctgtacaacgagctgaacctgggcagacgggaagagtacgacgtgctggataagcggcggggtcgggaccccgagatgggaggcaagcctagaaggaagaacccccaagagggcctttataatgagctgcagaaagataagatggccgaagcctacagcgagatcggcatgaagggcgagagacgcagaggcaaaggccacgacggcctgtaccaaggcctctccaccgccacaaaggacacctacgacgccctgcacatgcaggccctgcctccaagatga

SEQ ID NO: 59 - DNA, 1440 bp, Synthetic construct
  source 1..1440: mol_type = other DNA; note = Chimeric Antigen Receptor DNA #3 (OX40)
atggccctgcctgtgaccgccctgctgctgccactggccctgctgctgcacgccgctagacctgacatccagatgacacagaccaccagcagcctgtcagcttctctgggagatagagtgaccatcagctgcagagccagccaggacatcagcaactacctgaactggtatcagcagaagcctgatggcacagtgaagctgctgatctactacaccagccggctgcacagcggcgtgccatctagattcagcggaagcggcagcggcaccgactacagcctcacaatcagcaacctggaacaggaggacatcgccacctacttctgccagcagggcaacacactgccttggaccttcggcggcggcaccaagctggaaatcaagggcggaggcggcagcggcggcggaggctctggcggcggaggttccgaggtgcagctgcaacaaagcggcgccgagctggtgaagcccggcgccagcgtgaaactgagctgtaccgctagcggatttaacatcaaggacacatatatgcactgggtcaaacagcggcccgagcagggcctcgaatggatcggccggatcgaccctgccaacggcaatactcaatacgaccccaagttccagggcaaggccacaattcctgctgatacatcttctaataccgcctatctgcagctgagcagcctgacctccgaggataccgccgtgtactactgcgtgtacggcagaggctctgcctggttcgccttttggggccagggaaccctggttacggtgtccgccacaaccacccctgcccctagacctcctacccctgctccaaccatcgccagccagcccctgtctctgcggcctgaagcttgcagacccgctgctggcggcgccgtgcacacgaggggactggacttcgcctgcgacatctacatttgggcccctctggccggcacctgcggcgtgctgctgctgagcctggtcatcacactgtactgccggagggaccagaggctgccccccgatgcccacaagccccctgggggaggcagtttccggacccccatccaagaggagcaggccgacgcccactccaccctggccaagatcagagtgaagttcagcagaagcgccgatgcccctgcctaccagcagggacagaaccagctgtacaacgagctgaacctgggcagacgggaagagtacgacgtgctggataagcggcggggtcgggaccccgagatgggaggcaagcctagaaggaagaacccccaagagggcctttataatgagctgcagaaagataagatggccgaagcctacagcgagatcggcatgaagggcgagagacgcagaggcaaaggccacgacggcctgtaccaaggcctctccaccgccacaaaggacacctacgacgccctgcacatgcaggccctgcctccaagatga

(19) United States
(12) Patent Application Publication
(10) Pub. No.: US 2025/0101113 A1
(43) Pub. Date: Mar. 27, 2025

(54) ANTI-CD94 ANTIBODY AND CHIMERIC ANTIGEN RECEPTOR AND METHODS OF USE THEREOF

(71) Applicant: BOARD OF REGENTS, THE UNIVERSITY OF TEXAS SYSTEM, Austin, TX (US)

(72) Inventors: Sattva S. NEELAPU, Houston, TX (US); Jinsheng WENG, Houston, TX (US); Yong PAN, Houston, TX (US)

(21) Appl. No.: 18/728,332

(22) PCT Filed: Jan. 10, 2023
(86) PCT No.: PCT/US2023/060431

§ 371 Date: Jul. 11, 2024

Related U.S. Application Data
(60) Provisional application No. 63/298,423, filed on Jan. 11, 2022.

(51) Int. Cl. C07K 16/28; A61K 39/00; A61P 35/00; C07K 14/705; C07K 14/725; C12N 5/0783

(52) U.S. Cl. CPC C07K 16/2851; A61K 40/11; A61K 40/31; A61K 40/4224; A61P 35/00; C07K 14/7051; C07K 14/70517; C07K 14/70521; C07K 14/70578; C12N 5/0636; C07K 2317/53; C07K 2317/622; C07K 2319/02; C07K 2319/03; C12N 2510/00

(57) ABSTRACT

According to various aspects of this disclosure, the present disclosure relates to an antibody or antigen-binding fragment thereof capable of binding to CD94, a chimeric antigen receptor capable of binding to CD94, and engineered T cells containing chimeric antigen receptors capable of binding to CD94.

### CROSS REFERENCE TO RELATED APPLICATIONS

The present application claims the priority benefit of U.S. Provisional Application No. 63/298,423 filed Jan. 11, 2022, which is hereby incorporated by reference in its entirety.

### REFERENCE TO SEQUENCE LISTING SUBMITTED ELECTRONICALLY VIA EFS-WEB

The content of the electronically submitted sequence listing (Name: 4443_010PC01_Seqlisting_ST26.xml; Size: 73,637 bytes; and Date of Creation: Jan. 5, 2023), filed with the application, is incorporated herein by reference in its entirety.

### FIELD OF THE DISCLOSURE

The disclosure generally relates to the fields of immunology and cancer therapy. For example, the present disclosure provides anti-CD94 antibodies, anti-CD94 chimeric antigen receptors and anti-CD94 CAR-T cells, and methods of making and using the same.

### BACKGROUND

Chimeric antigen receptor (CAR) T-cell therapy has shown efficacy in B cell leukemias and lymphomas. However, targeting Natural Killer (NK) cell and T cell lymphoproliferative disorders has been challenging due to difficulty in identifying appropriate antigens for CAR-T cell therapy.

CD94 is a C-type lectin receptor expressed on NK cells and a subset of CD8+ T cells (<5%) amongst normal tissues. It can function as a stimulatory or inhibitory receptor when it heterodimerizes with NKG2C or NKG2A, respectively. Studies in patients with primary immunodeficiency where NK-cell deficiency is the major immunological defect indicate that they are primarily at risk of recurrent herpes virus infections, which can be controlled with a combination of vaccination, prophylaxis, and/or therapeutic strategies. Collectively, these reports suggest that CD94 could be a safe and effective target for CAR T-cell therapy in CD94-expressing NK/T-cell lymphoproliferative disorders.

There is a need to develop antibodies and CAR-T cells, e.g., for the treatment of Natural Killer cell and T cell lymphoproliferative disorders.

### BRIEF SUMMARY

The present disclosure provides an antibody or antigen-binding fragment thereof capable of binding to CD94 (e.g., an anti-CD94 specific antibody). In some aspects, the antibody or antigen-binding fragment thereof comprises a complementary determining region (CDR) H1 comprising the amino acid sequence set forth in SEQ ID NO: 3 or SEQ ID NO: 19, a CDR H2 comprising the amino acid sequence set forth in SEQ ID NO: 4 or SEQ ID NO: 20, a CDR H3 comprising the amino acid sequence set forth in SEQ ID NO: 5 or SEQ ID NO: 21, a CDR L1 comprising the amino acid sequence set forth in SEQ ID NO: 6 or SEQ ID NO: 22, a CDR L2 comprising the amino acid sequence YTS, SEQ ID NO: 23, or SEQ ID NO: 24, and a CDR L3 comprising the amino acid sequence set forth in SEQ ID NO: 8.

In some aspects, the antibody or antigen-binding fragment thereof of comprises a heavy chain variable region (VH) and a light chain variable region (VL). In some aspects, the VH comprises an amino acid sequence having at least 85%, at least 90%, at least 91% at least 92%, at least 93%, at least 94%, at least 95%, at least 96%, at least 97%, at least 98%, at least 99%, or 100% sequence identity to SEQ ID NO: 1 and the VL comprises an amino acid sequence having at least 85%, at least 90%, at least 91%, at least 92%, at least 93%, at least 94%, at least 95%, at least 96%, at least 97%, at least 98%, at least 99%, or 100% sequence identity to SEQ ID NO: 2.

In some aspects, the antibody or antigen-binding fragment thereof comprises: i) a CDR H1, CDR H2, and CDR H3 comprising the CDR H1, CDR H2, and CDR H3 amino acid sequences of SEQ ID NO: 1; and ii) a CDR L1, CDR L2, and CDR L3 comprising the CDR L1, CDR L2, and CDR L3 amino acid sequences of SEQ ID NO: 2.

In some aspects, the CDRs are the Kabat-defined CDRs, the Chothia-defined CDRs, the AbM-defined CDRs, or the IMGT-defined CDRs.

In some aspects, the antibody or antigen-binding fragment comprises a VH comprising an amino acid sequence at least 85% identical to the amino acid sequence SEQ ID NO: 1.

In some aspects, the antibody or antigen-binding fragment comprises a VH comprising an amino acid sequence at least 90% identical to the amino acid sequence SEQ ID NO: 1.

In some aspects, the antibody or antigen-binding fragment comprises a VH comprising an amino acid sequence at least 95% identical to the amino acid sequence SEQ ID NO: 1.

In some aspects, the antibody or antigen-binding fragment comprises a VH comprising an amino acid sequence at least 98% identical to the amino acid sequence SEQ ID NO: 1.

In some aspects, the antibody or antigen-binding fragment comprises a VH comprising an amino acid sequence at least 99% identical to the amino acid sequence SEQ ID NO: 1.

In some aspects, the antibody or antigen-binding fragment comprises a VL comprising an amino acid sequence at least 85% identical to the amino acid sequence of SEQ ID NO: 2.

In some aspects, the antibody or antigen-binding fragment comprises a VL comprising an amino acid sequence at least 90% identical to the amino acid sequence of SEQ ID NO: 2.

In some aspects, the antibody or antigen-binding fragment comprises a VL comprising an amino acid sequence at least 95% identical to the amino acid sequence of SEQ ID NO: 2.

In some aspects, the antibody or antigen-binding fragment comprises a VL comprising an amino acid sequence at least 98% identical to the amino acid sequence of SEQ ID NO: 2.

In some aspects, the antibody or antigen-binding fragment comprises a VL comprising an amino acid sequence at least 99% identical to the amino acid sequence of SEQ ID NO: 2.

In some aspects, the antibody or antigen-binding fragment thereof is human, humanized, or chimeric.

In some aspects, the antibody or antigen-binding fragment thereof is an IgG antibody.

In some aspects, the IgG antibody is an IgG1 antibody or an IgG4 antibody.

In some aspects, said antibody is an antigen-binding fragment of an antibody.

In some aspects, said fragment is selected from the group consisting of Fab, F(ab′)2, Fv, scFv, scFv-Fc, dsFv and a single domain molecule.

In some aspects, said fragment is a scFv.

In some aspects, said fragment is a Fab.

In some aspects, said fragment is an intrabody.

In some aspects, the antigen-binding fragment is devoid of an Fc region.

In some aspects, the antibody or antigen-binding fragment comprises a VH and a VL on the same polypeptide chain.

In some aspects, the VH and VL are connected by a linker.

In some aspects, the antibody or antigen-binding fragment thereof is conjugated to an agent selected from the group consisting of a therapeutic agent, a prodrug, a peptide, a protein, an enzyme, a virus, a lipid, a biological response modifier, a pharmaceutical agent, and PEG.

In some aspects, the antibody or antigen-binding fragment thereof is a bispecific antibody.

In some aspects, disclosed herein is a chimeric antigen receptor (CAR) which comprises, from N-terminus to C-terminus: (a) an extracellular ligand-binding domain comprising an antigen-binding domain capable of binding to CD94; (b) a hinge; (c) a transmembrane domain; and (d) a cytoplasmic domain comprising a costimulatory domain and a signaling domain. In some aspects, the antigen-binding domain comprises a CDR H1 comprising the amino acid sequence set forth in SEQ ID NO: 3 or SEQ ID NO: 19, a CDR H2 comprising the amino acid sequence set forth in SEQ ID NO: 4 or SEQ ID NO: 20, a CDR H3 comprising the amino acid sequence set forth in SEQ ID NO: 5 or SEQ ID NO: 21, a CDR L1 comprising the amino acid sequence set forth in SEQ ID NO: 6 or SEQ ID NO: 22, a CDR L2 comprising the amino acid sequence YTS or the amino acid sequence set forth in SEQ ID NO: 23 or SEQ ID NO: 24, and a CDR L3 comprising the amino acid sequence set forth in SEQ ID NO: 8.

In some aspects, the chimeric antigen receptor comprises a heavy chain variable region (VH) comprising the amino acid sequence of SEQ ID NO: 1 and a light chain variable region (VL) comprising the amino acid sequence of SEQ ID NO: 2.

In some aspects, the antigen-binding domain is a scFv.

In some aspects, the costimulatory domain comprises a 4-1BB costimulatory domain, a CD28 costimulatory domain, or an OX40 costimulatory domain.

In some aspects, the hinge, the transmembrane domain, or both, are from a CD8α polypeptide.

In some aspects, the signaling domain comprises a CD3zeta signaling domain.

In some aspects, disclosed herein is an engineered human T cell comprising a chimeric antigen receptor (CAR) comprising, from N-terminus to C-terminus: (a) an extracellular ligand-binding domain comprising a scFv domain capable of binding to CD94, wherein the scFv domain comprises a VL and a VH; (b) a hinge; (c) a transmembrane domain; and (d) a cytoplasmic domain comprising a costimulatory domain and a signaling domain. In some aspects, the VH comprises a CDR H1 comprising the amino acid sequence set forth in SEQ ID NO: 3 or SEQ ID NO: 19, a CDR H2 comprising the amino acid sequence set forth in SEQ ID NO: 4 or SEQ ID NO: 20, a CDR H3 comprising the amino acid sequence set forth in SEQ ID NO: 5 or SEQ ID NO: 21. In some aspects, the VL comprises a CDR L1 comprising the amino acid sequence set forth in SEQ ID NO: 6 or SEQ ID NO: 22, a CDR L2 comprising the amino acid sequence YTS or the amino acid sequence set forth in SEQ ID NO: 23 or SEQ ID NO: 24, and a CDR L3 comprising the amino acid sequence set forth in SEQ ID NO: 8.

In some aspects, the engineered human T cell comprises a chimeric antigen receptor (CAR) comprising an amino acid sequence having at least 85%, at least 90%, at least 91% at least 92%, at least 93%, at least 94%, at least 95%, at least 96%, at least 97%, at least 98%, at least 99%, or 100% sequence identity to SEQ ID NOs: 9, 56, or 57.

In some aspects, disclosed herein is an isolated polynucleotide comprising a nucleic acid molecule encoding the VH or heavy chain of any of the antibody or antigen-binding fragment thereof disclosed herein.

In some aspects, the isolated polynucleotide further comprises a nucleic acid molecule encoding any of the VL or light chain of the antibody or antigen-binding fragment thereof disclosed herein.

In some aspects, disclosed herein is an isolated polynucleotide comprising a nucleic acid molecule encoding any of the VL or light chain of the antibody or antigen-binding fragment thereof disclosed herein.

In some aspects, disclosed herein is an isolated polynucleotide comprising a nucleic acid molecule encoding any of the chimeric antigen receptor disclosed herein.

In some aspects, disclosed herein is an isolated vector comprising the any of the polynucleotides disclosed herein.

In some aspects, disclosed herein is a host cell comprising any of the polynucleotides disclosed herein or any of the vectors disclosed herein.

In some aspects, the host cell is selected form the group consisting of CHO, HEK-293T, HeLa and BHK cells, optionally wherein the CHO cell is a CHO-K1SP cell.

In some aspects, disclosed herein is a method of producing an antibody or antigen-binding fragment thereof capable of binding to CD94, the method comprising: (a) culturing any of the host cells disclosed herein in a cell culture under conditions which allow expression of the antibody or antigen-binding fragment thereof; and (b) recovering the antibody or antigen-binding fragment thereof from said cell culture.

In some aspects, disclosed herein is an antibody or antigen-binding fragment thereof obtainable by any of the methods of producing disclosed herein.

In some aspects, disclosed herein is a pharmaceutical composition comprising any of the antibody or antigen-binding fragments thereof disclosed herein, any of the chimeric antigen receptors disclosed herein, any of the engineered T cells disclosed herein, or any of the vectors disclosed herein. In some aspects, the pharmaceutical composition further comprises a pharmaceutically acceptable excipient.

In some aspects, disclosed herein is a method of treating cancer in an individual, the method comprising administering to the individual a therapeutically effective amount of any of the antibody or antigen-binding fragments thereof disclosed herein, any of the chimeric antigen receptors disclosed herein, any of the engineered T cells disclosed herein, or any of the pharmaceutical compositions disclosed herein.

In some aspects, the cancer is a leukemia. In some aspects, the cancer is a CD94 expressing cancer.

In some aspects, the leukemia is T cell leukemia, T cell large granular leukemia, Natural Killer cell large granular leukemia, or Natural Killer cell leukemia.

In some aspects, the cancer is a lymphoma.

In some aspects, the lymphoma is T cell lymphoma, extranodal Natural Killer/T cell lymphoma, hepatosplenic T cell lymphoma, angioimmunoblastic T cell lymphoma, or anaplastic large cell lymphoma.

In some aspects, described herein is a method of treating or preventing graft-rejection of a transplant in a patient, the method comprising administering to the individual therapeutically effective amount of any of the antibody or antigen-binding fragments thereof described herein, any of the chimeric antigen receptors described herein, any of the engineered T cells described herein, or any of the pharmaceutical compositions described herein.

In some aspects, the transplant is an allogenic transplant.

In some aspects, the transplant is an organ transplant.

In some aspects, the transplant is a hematopoietic cell transplant.

In some aspects, the transplant is an induced pluripotent cell therapy.

In some aspects, described herein is a method of modulating an immune response in a subject, the method comprising administering to the individual therapeutically effective amount of any of the antibody or antigen-binding fragments thereof described herein, any of the chimeric antigen receptors described herein, any of the engineered T cells disclosed herein, or any of the pharmaceutical compositions disclosed herein.

In some aspects, the immune response is enhanced.

In some aspects, the immune response is mediated by Natural Killer cells and/or T cells. In some aspects, the Natural Killer cells and/or T cells mediate the immune response of an autoimmune disease. In some aspects, the autoimmune disease is a Systemic Autoimmune Disease. In some aspects, the systemic autoimmune disease is systemic lupus erythematosus (SLE), Sjögren's Syndrome, Systemic Sclerosis, Rheumatoid Arthritis (RA), Multiple Sclerosis, type 1 diabetes mellitus (T1DM), or autoimmune liver disease (ALD).

### DESCRIPTION OF FIGURES

FIG. 1A shows expression of CD94 mRNA relative to β-actin mRNA in the Natural Killer (NK) cell lymphoma cell line NK-92 and normal human tissues.

FIG. 1B shows expression of CD94 mRNA in 79 normal human tissues and lymphoma subtypes by high-density oligonucleotide arrays from BioGPS.

FIG. 2 shows a flow cytometry analysis indicating generation of CD94-L cells by measuring positive staining for CD94 (x-axis).

FIG. 3A-3C show the specificity of a purified anti-CD94 monoclonal antibody generated by hybridoma technology (“UT-CD94”). FIG. 3A shows staining of CD94-L cells (CD94-L) and L cells (L) by the UT-CD94 monoclonal antibody conjugated to AF647. FIG. 3B shows staining of CD56+ NK cells (Nk cells), CD3+ T cells (T cells), CD19+ B cells (B cells), and CD14+ monocytes (Monocytes) from normal donor peripheral blood mononuclear cells by the UT-CD94 monoclonal antibody conjugated to AF647. FIG. 3C shows staining of NK lymphoma or leukemia lines (NK-92, NKL, and KHYG-1) and B cell lymphoma lines (SP53, Daudi, and Jeko-1) by the UT-CD94 monoclonal antibody conjugated to AF647.

FIG. 4A illustrates an exemplary design of an anti-CD94 Chimeric Antigen Receptor (“UT-CD94 CAR”). The UT-CD94 CAR includes (i) a CD94 scFv including a VL and VH domain, (ii) a CD8α hinge domain, (iii) a CD8α transmembrane domain, (iv) a 4-1BB costimulatory domain, and (v) a CD3zeta signaling domain.

FIG. 4B shows the expression of the UT-CD94 CAR 72 hours after lentiviral transduction as determined by staining with a His-tagged CD94 extracellular domain (ECD) protein and an AF647-conjugated anti-His antibody. Flow cytometry analysis indicates positive staining for the UT-CD94 CAR on the x-axis.

FIG. 5A-5F show the cytotoxicity of anti-CD94 CAR-T cells (“UT-CD94 CAR-T cells”). FIGS. 5A-5B show the lysis percentage of CD94 positive NK cell lines NKL (FIG. 5A) or NK-92 (FIG. 5B) at Effector:Target ratios of 8:1, 4:1, and 1:1 after co-culture with UT-CD94 CAR-T cells (CD94 CAR-T), co-culture with untransduced T cells (Control T), or no co-culture (Tumor Only). FIGS. 5C-E show a 4 day period with the corresponding lysis percentage of CD94 positive NK cell line NKL (FIG. 5C), CD94 positive NK cell line NK-92 (FIG. 5D), or B cell lymphoma cell line SP53 (FIG. 5E) after co-culture with UT-CD94 CAR-T cells (CD94 CAR-T), co-culture with untransduced T cells (Control T), or no co-culture (Tumor Only). FIG. 5F shows percentage of dead target cells in L cells, human CD94-transfected L cells, B cell lymphoma cell line SP53, and NK92 cell line when cultured alone or in combination with untransduced T cells, anti-CD94 CAR-T cells or anti-CD19 CAR-T cells at an effector:target ratio of 6:1 for 24 hours.

FIG. 6 shows a flow cytometry analysis measuring the effect of a chimeric anti-CD94 antibody (“cUT-CD94”) or isotype antibody on the staining of HLA-E tetramers (as measured by Median Fluorescence Intensity).

FIG. 7 shows flow cytometry analysis measuring CD137+ NK cells after incubation with HLA-E expressing SP-53 mantle cell lymphoma cells and treatment with murine anti-human CD94 antibodies (UT-CD94/mCD94 and DX22 from Biolegend), cUT-CD94 antibody (cCD94), and relevant isotype controls (mIgG1 and hIgG1 from Biolegend).

FIG. 8 shows flow cytometry analysis measuring CD107a+ NK cells after incubation with HLA-E expressing SP-53 mantle cell lymphoma cells and treatment with murine anti-human CD94 antibodies (UT-CD94/mCD94 and DX22 from Biolegend), cUT-CD94 antibody (cCD94), and relevant isotype controls (mIgG1 and hIgG1 from Biolegend).

FIGS. 9A-9G show scRNAseq analysis of CD94 (KLRD1) mRNA expression. Overview of CD94 mRNA expression was based on scRNAseq analysis across 25 normal human tissues (FIG. 9A), in single cell types from all 25 tissues (FIG. 9B), heart (FIG. 9C), lung (FIG. 9D), liver (FIG. 9E), and kidney (FIG. 9F). Additionally, CD94 mRNA expression in 29 immune cell types from normal donor peripheral blood was analyzed (FIG. 9G). nTPM—normalized transcripts per million. Source: Human Protein Atlas.

FIGS. 10A-10C show an immunohistochemistry assay for CD94 expression in normal donor tissue array (FIG. 10A), normal tonsil (FIG. 10B), and tumor biopsy from a subject with NK/T-cell lymphoma (FIG. 10C).

FIG. 11 shows binding of anti-CD94 antibody in wild-type and CD94 knock-out NK-cell leukemia cell lines. The binding specificity of anti-CD94 antibody was assessed by flow cytometry against wild-type KHYG-1 and NK92 cell lines and their isogenic cell lines with CD94 knock-out (KO).

FIGS. 12A-12E show exemplary CD94-targeting CAR constructs and their cytotoxic activity in vitro. FIG. 12A shows the design of three different CD94-targeting CARs. FIG. 12B shows flow cytometry assessment of CD94-targeting CARs were lentivirally transduced into normal donor T cells. FIGS. 12C-12E shows the percentage of cells that underwent lysis or growth as monitored by serial imaging using the Incucyte Live-Cell Analysis System. NK-cell leukemia cell lines (NKL-FIG. 12C, KHYG-1-FIG. 12D, and NK92-FIG. 12E) were stably transduced with red fluorescent protein (RFP). The tumor cells were then cultured in the presence or absence of CD94-targeting CAR T cells or untransduced T cells at an Effector:Target ratio of 0.5:1.

FIGS. 13A-13C show the expression of CD94 in hepatosplenic T-cell lymphoma and cytotoxicity of anti-CD94 against PDX tumor cells. FIG. 13A shows a picture of liver and spleen at necropsy of NSG mice that were injected intravenously with hepatosplenic T-cell lymphoma (TCL) PDX cells (1×10⁶ tumor cells/mouse). FIG. 13B shows CD3 (hCD3), CD94 (hCD94), and CD45 (hCD45) expression in liver, spleen, bone marrow, and blood as measured by flow cytometry between hCD3+hCD45+ cells vs. hCD3-hCD45-cells. NSG mice that were not injected with tumor were used as controls. FIG. 13C shows survival in 5 different mouse cohorts: 1) tumor only, 2) untransduced T cells, 3) mice administered with 10×10⁶ anti-CD94-targeting CAR+ T cells (CD94/CD28), 4) mice administered with 10×10⁶ anti-CD94-targeting CAR+ T cells (CD94/4-1BB), 5) mice administered with 10×10⁶ anti-CD94-targeting CAR+ T cells (CD94/OX40). Antitumor effects of CAR T cells was assessed by monitoring the mice for survival. Log-rank test was used to evaluate the difference in survival between treatment groups.

### DETAILED DESCRIPTION

Certain aspects of the disclosure are directed to anti-CD94 antibodies, anti-CD94 chimeric antigen receptors and anti-CD94 CAR-T cells, and methods of making and using the same. In some aspects, disclosed herein is an antibody or antigen-binding fragment thereof capable of binding to CD94. In some aspects, the antibody or antigen-binding fragment thereof comprises a heavy chain variable region (VH) comprising complementary determining region (CDR) H1 comprising the amino acid sequence set forth in SEQ ID NO: 3 or SEQ ID NO: 19, a CDR H2 comprising the amino acid sequence set forth in SEQ ID NO: 4 or SEQ ID NO: 20, and a CDR H3 comprising the amino acid sequence set forth in SEQ ID NO: 5 or SEQ ID NO: 21 and a light chain variable region (VL) comprising a CDR L1 comprising the amino acid sequence set forth in SEQ ID NO: 6 or SEQ ID NO: 22, a CDR L2 comprising the amino acid sequence YTS or the amino acid sequence set forth in SEQ ID NO: 23 or SEQ ID NO: 24, and a CDR L3 comprising the amino acid sequence set forth in SEQ ID NO: 8. In some aspects, the antibody or antigen-binding fragment thereof comprises a heavy chain variable region (VH) comprising an amino acid sequence having at least 85%, at least 90%, at least 91%, at least 92%, at least 93%, at least 94%, at least 95%, at least 96%, at least 97%, at least 98%, at least 99%, or 100% sequence identity to SEQ ID NO: 1 and a light chain variable region (VL) comprising an amino acid sequence having at least 85%, at least 90%, at least 91%, at least 92%, at least 93%, at least 94%, at least 95%, at least 96%, at least 97%, at least 98%, at least 99%, or 100% sequence identity to SEQ ID NO: 2. In some aspects, the antibody or antigen-binding fragment thereof comprises an anti-CD94 scFv comprising the VL and VH domains. In some aspects, disclosed herein is a chimeric antigen receptors (CAR) or CAR T-cell comprising an anti-CD94 antibody or antigen-binding fragment thereof (e.g., an anti-CD94 scFv) disclosed herein.

### I. Definitions

Unless defined otherwise, all technical and scientific terms used herein have the same meaning as commonly understood by one of ordinary skill in the art to which this disclosure belongs. In case of conflict, the present application including the definitions will control. Unless otherwise required by context, singular terms shall include pluralities and plural terms shall include the singular. All publications, patents and other references mentioned herein are incorporated by reference in their entireties for all purposes as if each individual publication or patent application were specifically and individually indicated to be incorporated by reference.

Although methods and materials similar or equivalent to those disclosed herein can be used in practice or testing of the present disclosure, suitable methods and materials are disclosed below. The materials, methods and examples are illustrative only and are not intended to be limiting. Other features and advantages of the disclosure will be apparent from the detailed description and from the claims.

In order to further define this disclosure, the following terms and definitions are provided.

The singular forms “a,” “an” and “the” include plural referents unless the context clearly dictates otherwise. The terms “a” (or “an”), as well as the terms “one or more,” and “at least one” can be used interchangeably herein. In certain aspects, the term “a” or “an” means “single.” In other aspects, the term “a” or “an” includes “two or more” or “multiple.”

The term “about” is used herein to mean approximately, roughly, around, or in the regions of. When the term “about” is used in conjunction with a numerical range, it modifies that range by extending the boundaries above and below the numerical values set forth. In general, the term “about” is used herein to modify a numerical value above and below the stated value by a variance of 10 percent, up or down (higher or lower).

Throughout this disclosure, various aspects of this disclosure are presented in a range format. Numeric ranges are inclusive of the numbers defining the range. Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither or both limits are included is also encompassed within the disclosure. Thus, ranges recited herein are understood to be shorthand for all of the values within the range, inclusive of the recited endpoints. For example, a range of 1 to 10 is understood to include any number, combination of numbers, or sub-range from the group consisting of 1, 2, 3, 4, 5, 6, 7, 8, 9, and 10.

Where a value is explicitly recited, it is to be understood that values which are about the same quantity or amount as the recited value are also within the scope of the disclosure. Where a combination is disclosed, each subcombination of the elements of that combination is also specifically disclosed and is within the scope of the disclosure. Conversely, where different elements or groups of elements are individually disclosed, combinations thereof are also disclosed. Where any element of a disclosure is disclosed as having a plurality of alternatives, examples of that disclosure in which each alternative is excluded singly or in any combination with the other alternatives are also hereby disclosed; more than one element of a disclosure can have such exclusions, and all combinations of elements having such exclusions are hereby disclosed.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The term “pharmaceutically acceptable” as used herein refers to those compounds, materials, compositions, formulations, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “excipient” refers to any substance, not itself a therapeutic agent, which can be used in a composition for delivery of an active therapeutic agent to a subject or combined with an active therapeutic agent (e.g., to create a pharmaceutical composition) to improve its handling or storage properties or to permit or facilitate formation of a dose unit of the composition (e.g., formation of a hydrogel which can then be optionally incorporated into a patch). Excipients include, but are not limited to, solvents, penetration enhancers, wetting agents, antioxidants, lubricants, emollients, substances added to improve appearance or texture of the composition and substances used to form hydrogels. Any such excipients can be used in any dosage forms according to the present disclosure. The foregoing classes of excipients are not meant to be exhaustive but merely illustrative as a person of ordinary skill in the art would recognize that additional types and combinations of excipients could be used to achieve the desired goals for delivery of a drug. The excipient can be an inert substance, an inactive substance, and/or a not medicinally active substance. The excipient can serve various purposes. A person skilled in the art can select one or more excipients with respect to the particular desired properties by routine experimentation and without any undue burden. The amount of each excipient used can vary within ranges conventional in the art. Techniques and excipients which can be used to formulate dosage forms are described in Handbook of Pharmaceutical Excipients, 6th edition, Rowe et al., Eds., American Pharmaceuticals Association and the Pharmaceutical Press, publications department of the Royal Pharmaceutical Society of Great Britain (2009); and Remington: the Science and Practice of Pharmacy, 21st edition, Gennaro, Ed., Lippincott Williams & Wilkins (2005).

The term “effective amount” or “pharmaceutically effective amount” or “therapeutically effective amount” as used herein refers to the amount or quantity of a drug or pharmaceutically active substance which is sufficient to elicit the required or desired therapeutic response, or in other words, the amount which is sufficient to elicit an appreciable biological response when administered to a patient.

The term “treating” or “treatment” as used herein refers to the administration of a composition to a subject for therapeutic purposes.

The term “antibody” means an immunoglobulin molecule that recognizes and specifically binds to a target, such as a protein, polypeptide, peptide, carbohydrate, polynucleotide, lipid, or combinations of the foregoing through at least one antigen recognition site within the variable region of the immunoglobulin molecule. As used herein, the term “antibody” encompasses intact polyclonal antibodies, intact monoclonal antibodies, chimeric antibodies, humanized antibodies, human antibodies, fusion proteins comprising an antibody, and any other modified immunoglobulin molecule so long as the antibodies exhibit the desired biological activity. An antibody can be of any the five major classes of immunoglobulins: IgA, IgD, IgE, IgG, and IgM, or subclasses (isotypes) thereof (e.g. IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well known subunit structures and three-dimensional configurations. Antibodies can be naked or conjugated to other molecules such as toxins, radioisotopes, etc.

The term “antibody fragment” refers to a portion of an intact antibody. An “antigen-binding fragment,” “antigen-binding domain,” or “antigen-binding region,” refers to a portion of an intact antibody that binds to an antigen. An antigen-binding fragment can contain an antigen recognition site of an intact antibody (e.g., complementarity determining regions (CDRs) sufficient to bind antigen). Examples of antigen-binding fragments of antibodies include, but are not limited to Fab, Fab′, F(ab′)2, and Fv fragments, linear antibodies, and single chain antibodies. An antigen-binding fragment of an antibody can be derived from any animal species, such as rodents (e.g., mouse, rat, or hamster) and humans or can be artificially produced.

As used herein, the terms “variable region” or “variable domain” are used interchangeably and are common in the art. The variable region typically refers to a portion of an antibody, generally, a portion of a light or heavy chain, typically about the amino-terminal 110 to 120 amino acids or 110 to 125 amino acids in the mature heavy chain and about 90 to 115 amino acids in the mature light chain, which differ in sequence among antibodies and are used in the binding and specificity of a particular antibody for its particular antigen. The variability in sequence is concentrated in those regions called complementarity determining regions (CDRs) while the more highly conserved regions in the variable domain are called framework regions (FR). Without wishing to be bound by any particular mechanism or theory, it is believed that CDRs of the light and heavy chains are primarily responsible for the interaction and specificity of the antibody with antigen.

The terms “VH” and “VH domain” are used interchangeably to refer to the heavy chain variable region of an antibody or antigen-binding fragment thereof.

The terms “VL” and “VL domain” are used interchangeably to refer to the light chain variable region of an antibody or antigen-binding fragment thereof.

The “hypervariable regions” in each chain are held together in close proximity by FRs, and with the hypervariable regions from the other chain, contribute to the formation of the antigen-binding site of antibodies (see Kabat et al, Sequences of Proteins of Immunological Interest, 1992). The term “hypervariable region” as used herein refers to the amino acid residues of an antibody, which are responsible for antigen binding. The hypervariable region generally comprises amino acid residues from a “complementary determining region” or “CDR”, the latter being of highest sequence variability and/or involved in antigen recognition. A number of CDR definitions are in use and are encompassed herein. The Kabat definition is based on sequence variability and is the most commonly used (Kabat E A et al., supra). Chothia refers instead to the location of the structural loops (Chothia C & Lesk A M (1987) J. Mol. Biol. 196:901-917). The AbM definition is a compromise between the Kabat and the Chothia definitions and is used by Oxford Molecular's AbM antibody modelling software (Martin A C R et al., (1989) Proc. Natl Acad. Sci. USA, 86:9268-72; Martin A C R et al., (1991) Methods Enzymol. 203:121-153; Pedersen J T et al., (1992) Immunomethods, 1:126-136; Rees A R et al., (1996) In Sternberg M. J. E. (ed.), Protein Structure Prediction. Oxford University Press, Oxford, 141-172). The contact definition has been recently introduced (Maccallum R M et al., (1996) J. Mol. Biol. 262:732-7 45) and is based on an analysis of the complex structures available in the Protein Databank. The definition of the CDR by IMGT®, the international ImMunoGeneTics information System® (http://www.imgt.org) is based on the IMGT numbering for all immunoglobulin and T cell receptor V-REGIONs of all species (IMGT®, the international ImMunoGeneTics information System®; Lefranc M P et al., (1991) Nucleic Acids Res. 27 (1): 209-12; Ruiz M et al., (2000) Nucleic Acids Res. 28 (1): 219-21; Lefranc M P (2001) Nucleic Acids Res. 29 (1): 207-9; Lefranc M P (2003) Nucleic Acids Res. 31 (1): 307-10; Lefranc M P et al., (2005) Dev. Comp. Immunol. 29 (3): 185-203; Kaas Q et al., (2007) Briefings in Functional Genomics & Proteomics, 6 (4): 253-64).

In some aspects, the Complementarity Determining Regions (CDRs) disclosed herein are defined according to IMGT®. In some aspects, the CDRs are defined according to Chothia. In some aspects, the CDRs are defined according to Kabat. For example, for the light chains, the variable domain residues for each of the CDRs can be (numbering according to Kabat E A, et al., supra): LCDR1: 27-32, LCDR2: 50-52, LCDR3: 89-97. The “non-CDR region” of the VL region as used herein comprise the amino acid sequences: 1-26 (FR1), 33-49 (FR2), 53-88 (FR3), and 98-approximately 107 (FR4). For the heavy chains, the variable domain residues for each of the three CDRs can be HCDR1: 26-35, HCDR2: 51-57 and HCDR3: 93-102. The “non-CDR region” of the VH region as used herein can comprise the amino acid sequences: 1-25 (FR1), 36-50 (FR2), 58-92 (FR3), and 103 to approximately 113 (FR4).

The CDRs of the present disclosure can comprise “extended CDRs” which are based on the aforementioned definitions and can have variable domain residues as follows: LCDR1: 24-36, LCDR2: 46-56, LCDR3: 89-97, HCDR1: 26-36, HCDR2: 47-65, HCDR3: 93-102. These extended CDRs are numbered as well according to Kabat et al., supra.

In some aspects, alternate CDR sequences are provided for the same framework amino acid sequences of a variable region. In some aspects, the alternate CDR sequences are generated using software programs. In some aspects, different software can be used to generate alternate CDR sequences for the framework sequences of a variable region with different CDR sequences resulting from the use of the different software programs. In some aspects, the use of alternate CDR sequences can improve binding affinities of a bispecific antibody to at least one antigen. In some aspects, alternate CDR sequences are used for affinity optimization of one or both antigen binding sites of a bispecific antibody according to the present invention. In some aspects, the alternate CDRs are defined according to Kabat, Chothia, Paratome, AbM, Contact and/or IMGT annotations. In some aspects, the CDRs are defined according to more than one annotation.

The “non-extended CDR region” of the VL region can comprise the amino acid sequences: 1-23 (FRI), 37-45 (FR2), 57-88 (FR3), and 98 to approximately 107 (FR4). The “non-extended CDR region” of the VH region can comprise the amino acid sequences: 1-25 (FRI), 37-46 (FR2), 66-92 (FR3), and 103 to approximately 113 (FR4).

As used herein, the term “Fab region” refers to VH and CHI domains of a heavy chain (“Fab heavy chain”), or VL and CL domains of a light chain (“Fab light chain”) of an immunoglobulin.

As used herein, the term “scFv” or “single chain antibody fragment” refers to a single chain consisting of a heavy chain variable region and a light chain variable region of an antibody being linearly linked together by a linker (e.g., a short peptide of 10-25 amino acids), which exhibits specific binding to an antigen.

As used herein, the terms “constant region” and “constant domain” are interchangeable and have their meaning common in the art. The constant region is an antibody portion, e.g., a carboxyl terminal portion of a light and/or heavy chain which is not directly involved in binding of an antibody to antigen but which can exhibit various effector functions, such as interaction with the Fc receptor. The constant region of an immunoglobulin molecule generally has a more conserved amino acid sequence relative to an immunoglobulin variable domain.

As used herein, the term “heavy chain” when used in reference to an antibody can refer to any distinct type, e.g., alpha (a), delta (d), epsilon (e), gamma (g), and mu (m), based on the amino acid sequence of the constant domain, which give rise to IgA, IgD, IgE, IgG, and IgM classes of antibodies, respectively, including subclasses of IgG, e.g., IgG1, IgG2, IgG3, and IgG4. Heavy chain amino acid sequences are well known in the art. In some aspects, the heavy chain is a human heavy chain.

As used herein, the term “light chain” when used in reference to an antibody can refer to any distinct type, e.g., kappa (κ) or lambda (l) based on the amino acid sequence of the constant domains. Light chain amino acid sequences are well known in the art. In some aspects, the light chain is a human light chain.

The term “chimeric” antibodies or antigen-binding fragments thereof refers to antibodies or antigen-binding fragments thereof wherein the amino acid sequence is derived from two or more species. Typically, the variable region of both light and heavy chains corresponds to the variable region of antibodies or antigen-binding fragments thereof derived from one species of mammals (e.g. mouse, rat, rabbit, etc.) with the desired specificity, affinity, and capability while the constant regions are homologous to the sequences in antibodies or antigen-binding fragments thereof derived from another (usually human) to avoid eliciting an immune response in that species.

The term “humanized” antibody or antigen-binding fragment thereof refers to forms of non-human (e.g. murine) antibodies or antigen-binding fragments that are specific immunoglobulin chains, chimeric immunoglobulins, or fragments thereof that contain minimal non-human (e.g., murine) sequences. Typically, humanized antibodies or antigen-binding fragments thereof are human immunoglobulins in which residues from the complementary determining region (CDR) are replaced by residues from the CDR of a non-human species (e.g. mouse, rat, rabbit, hamster) that have the desired specificity, affinity, and capability (“CDR grafted”) (Jones et al., Nature 321:522-525 (1986); Riechmann et al., Nature 332:323-327 (1988); Verhoeyen et al., Science 239:1534-1536 (1988)). In some instances, the Fv framework region (FR) residues of a human immunoglobulin are replaced with the corresponding residues in an antibody or fragment from a non-human species that has the desired specificity, affinity, and capability. The humanized antibody or antigen-binding fragment thereof can be further modified by the substitution of additional residues either in the Fv framework region and/or within the non-human CDR residues to refine and optimize the specificity, affinity, and/or capability of the antibody or antigen-binding fragment thereof. In general, the humanized antibody or antigen-binding fragment thereof will comprise substantially all of at least one, and typically two or three, variable domains containing all or substantially all of the CDR regions that correspond to the non-human immunoglobulin whereas all or substantially all of the FR regions are those of a human immunoglobulin consensus sequence. The humanized antibody or antigen-binding fragment thereof can also comprise at least a portion of an immunoglobulin constant region or domain (Fc), typically that of a human immunoglobulin. Examples of methods used to generate humanized antibodies are described in U.S. Pat. No. 5,225,539; Roguska et al., Proc. Natl. Acad. Sci., USA, 91(3):969-973 (1994), and Roguska et al., Protein Eng. 9(10):895-904 (1996). In some aspects, a “humanized antibody” is a resurfaced antibody.

The term “human” antibody or antigen-binding fragment thereof means an antibody or antigen-binding fragment thereof having an amino acid sequence derived from a human immunoglobulin gene locus, where such antibody or antigen-binding fragment is made using any technique known in the art. This definition of a human antibody or antigen-binding fragment thereof includes intact or full-length antibodies and fragments thereof.

“Binding affinity” generally refers to the strength of the sum total of non-covalent interactions between a single binding site of a molecule (e.g., an antibody or antigen binding fragment thereof) and its binding partner (e.g., an antigen). Unless indicated otherwise, as used herein, “binding affinity” refers to intrinsic binding affinity which reflects a 1:1 interaction between members of a binding pair (e.g., antibody or antigen binding fragment thereof and antigen). The affinity of a molecule X for its partner Y can generally be represented by the dissociation constant (KD). Affinity can be measured and/or expressed in a number of ways known in the art, including, but not limited to, equilibrium dissociation constant (KD), and equilibrium association constant (KA). The KD is calculated from the quotient of k_{off}/kₒₙ, whereas KA is calculated from the quotient of kₒₙ/k_{off}. kₒₙ refers to the association rate constant of, e.g., an antibody or antigen binding fragment thereof to an antigen, and k_{off} refers to the dissociation of, e.g., an antibody or antigen-binding fragment thereof from an antigen. The kₒₙ and k_{off} can be determined by techniques known to one of ordinary skill in the art, such as BIAcore® or KinExA.

As used herein, an “epitope” is a term in the art and refers to a localized region of an antigen to which an antibody or antigen-binding fragment thereof can specifically bind. An epitope can be, for example, contiguous amino acids of a polypeptide (linear or contiguous epitope) or an epitope can, for example, come together from two or more non-contiguous regions of a polypeptide or polypeptides (conformational, non-linear, discontinuous, or non-contiguous epitope). In certain aspects, the epitope to which an antibody or antigen-binding fragment thereof binds can be determined by, e.g., NMR spectroscopy, X-ray diffraction crystallography studies, ELISA assays, hydrogen/deuterium exchange coupled with mass spectrometry (e.g., liquid chromatography electrospray mass spectrometry), array-based oligo-peptide scanning assays, and/or mutagenesis mapping (e.g., site-directed mutagenesis mapping). For X-ray crystallography, crystallization can be accomplished using any of the known methods in the art (e.g., Giege R et al, (1994) Acta Crystallogr D Biol Crystallogr 50 (Pt 4): 339-350; McPherson A (1990) Eur J Biochem 189:1-23; Chayen N E (1997) Structure 5:1269-1274; McPherson A (1976) J Biol Chem 251: 6300-6303). Antigen crystals can be studied using well known X-ray diffraction techniques and can be refined using computer software such as X-PLOR (Yale University, 1992, distributed by Molecular Simulations, Inc.; see, e.g., Meth Enzymol (1985) volumes 114 & 115, eds Wyckoff H W et al.; U.S. 2004/0014194), and BUSTER (Bricogne G (1993) Acta Crystallogr D Biol Crystallogr 49 (Pt 1): 37-60; Bricogne G (1997) Meth Enzymol 276A: 361-423, ed Carter C W; Roversi P et al, (2000) Acta Crystallogr D Biol Crystallogr 56 (Pt 10): 1316-1323). Mutagenesis mapping studies can be accomplished using any method known to one of skill in the art. See, e.g., Champe M et al, (1995) J Biol Chem 270:1388-1394 and Cunningham B C & Wells J A (1989) Science 244:1081-1085 for a description of mutagenesis techniques, including alanine scanning mutagenesis techniques.

An antibody is said to “competitively inhibit” binding of a reference antibody to a given epitope if it preferentially binds to that epitope or an overlapping epitope to the extent that it blocks, to some degree, binding of the reference antibody to the epitope. Competitive inhibition can be determined by any method known in the art, for example, competition ELISA assays. An antibody can be said to competitively inhibit binding of the reference antibody to a given epitope by at least 90%, at least 80%, at least 70%, at least 60%, or at least 50%.

A polypeptide, antibody, polynucleotide, vector, cell, or composition which is “isolated” refers to a polypeptide, antibody, polynucleotide, vector, cell, or composition which is in a form not found in nature. Isolated polypeptides, antibodies, polynucleotides, vectors, cell or compositions include those which have been purified to a degree that they are no longer in a form in which they are found in nature. In some aspects, an antibody, polynucleotide, vector, cell, or composition which is isolated is substantially pure. As used herein, “substantially pure” refers to material which is at least 50% pure (i.e., free from contaminants), at least 90% pure, at least 95% pure, at least 98% pure, or at least 99% pure.

The terms “polypeptide,” “peptide,” and “protein” are used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that, because the polypeptides of this disclosure are based upon antibodies, in certain aspects, the polypeptides can occur as single chains or associated chains.

“Percent identity” refers to the extent of identity between two sequences (e.g., amino acid sequences or nucleic acid sequences). Percent identity can be determined by aligning two sequences, introducing gaps to maximize identity between the sequences. Alignments can be generated using programs known in the art. For purposes herein, alignment of nucleotide sequences can be performed with the blastn program set at default parameters, and alignment of amino acid sequences can be performed with the blastp program set at default parameters (see National Center for Biotechnology Information (NCBI) on the worldwide web, ncbi.nlm.nih.gov).

As used herein, the term “host cell” can be any type of cell, e.g., a primary cell, a cell in culture, or a cell from a cell line. In specific aspects, the term “host cell” refers to a cell transfected with a nucleic acid molecule and the progeny or potential progeny of such a cell. Progeny of such a cell may not be identical to the parent cell transfected with the nucleic acid molecule, e.g., due to mutations or environmental influences that may occur in succeeding generations or integration of the nucleic acid molecule into the host cell genome.

The term “pharmaceutical composition” refers to a preparation which is in such form as to permit the biological activity of the active ingredient to be effective, and which contains no additional components which are unacceptably toxic to a subject to which the composition would be administered. The composition can be sterile.

The terms “administer,” “administering,” “administration,” and the like, as used herein, refer to methods that can be used to enable delivery of a drug, e.g., an anti-CD94 antibody or antigen-binding fragment thereof, to the desired site(s) of biological action (e.g., intravenous administration). Administration techniques that can be employed with the agents and methods described herein are found in e.g., Goodman and Gilman, The Pharmacological Basis of Therapeutics, current edition, Pergamon; and Remington's, Pharmaceutical Sciences, current edition, Mack Publishing Co., Easton, Pa.

As used herein, the terms “subject” and “patient” are used interchangeably. The subject can be an animal. In some aspects, the subject is a mammal such as a non-human animal (e.g., cow, pig, horse, cat, dog, rat, mouse, monkey or other primate, etc.). In some aspects, the subject is a human.

As used herein, the terms “nucleic acid” or “polynucleotides” refers to nucleotides and/or polynucleotides, such as deoxyribonucleic acid (DNA) or ribonucleic acid (RNA), oligonucleotides, fragments generated by the polymerase chain reaction (PCR), and fragments generated by any of ligation, scission, endonuclease action, and exonuclease action. Nucleic acid molecules can be composed of monomers that are naturally-occurring nucleotides (such as DNA and RNA), or analogs of naturally-occurring nucleotides (e.g., enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Nucleic acids can be either single stranded or double stranded.

The term “chimeric antigen receptor” (CAR) refers to a chimeric molecule that includes a binding domain which binds to a component (e.g., a ligand) present on a target cell (for example, the binding domain can include an antibody or antigen-binding fragment thereof specific for a desired antigen (e.g., CD94)) and a T cell receptor-activating intracellular domain. In some aspects, the CAR exhibits a specific anti-target cellular immune activity. In certain aspects, CARs can include an extracellular ligand-binding domain (e.g., a single chain antibody-binding domain (scFv)) fused to the intracellular signaling domain of the T cell antigen receptor complex zeta chain. In some aspects, the CAR can include an extracellular ligand-binding domain, a hinge, a transmembrane domain, and a cytoplasmic domain. In some aspects, the cytoplasmic domain comprises a costimulatory domain (e.g., 4-1BB, CD28, or OX40) and a signaling domain (e.g., CD3zeta). In some aspects, the CAR, when expressed in T cells, can redirect antigen recognition based on the antibody's specificity.

The term “vector,” as used herein, includes, but is not limited to, a viral vector, a plasmid, an RNA vector or a linear or circular DNA or RNA molecule which can include chromosomal, non-chromosomal, semi-synthetic or synthetic nucleic acids. In some cases, the vectors are those capable of autonomous replication (episomal vector) and/or expression of nucleic acids to which they are linked (expression vectors). Large numbers of suitable vectors are known to those of skill in the art and are commercially available. Viral vectors include retrovirus, adenovirus, parvovirus (e.g., adenoassociated viruses), coronavirus, negative strand RNA viruses such as orthomyxovirus (e.g., influenza virus), rhabdovirus (e.g., rabies and vesicular stomatitis virus), paramyxovirus (e.g. measles and Sendai), positive strand RNA viruses such as picornavirus and alphavirus, and double-stranded DNA viruses including adenovirus, herpesvirus (e.g., Herpes Simplex virus types 1 and 2, Epstein-Barr virus, cytomegalovirus), and poxvirus (e.g., vaccinia, fowlpox and canarypox). Other viruses include Norwalk virus, togavirus, flavivirus, reoviruses, papovavirus, hepadnavirus, and hepatitis virus, for example. Examples of retroviruses include: avian leukosis-sarcoma, mammalian C-type, B-type viruses, D type viruses, HTLV-BLV group, and lentivirus.

A “costimulatory domain” or “costimulatory molecule” refers to the cognate binding partner on a T-cell that specifically binds with a costimulatory ligand, thereby mediating a costimulatory response by the cell (e.g., a proliferation response). Costimulatory molecules include, but are not limited to, an MHC class I molecule, BTLA and Toll ligand receptor. Examples of costimulatory molecules include CD27, CD28, CD8, 4-1BB (CD137), OX40, CD30, CD40, PD-1, ICOS, lymphocyte function-associated antigen-1 (LFA-1), CD2, CD7, LIGHT, NKG2C, B7-H3 and a ligand that specifically binds with CD83 and the like. In some aspects, the costimulatory molecule is a cell surface molecule, other than an antigen receptor or their ligands, that is promotes an efficient immune response.

A “costimulatory ligand” refers to a molecule on an antigen presenting cell that specifically binds a cognate costimulatory molecule on a T-cell. In some aspects, the costimulatory ligand provides a signal which, in addition to the primary signal provided by, for instance, binding of a TCR/CD3 complex with an MHC molecule loaded with peptide, mediates a T cell response. In some aspects, the T cell response includes, but is not limited to, proliferation activation, differentiation and the like. A costimulatory ligand can include but is not limited to CD7, B7-1 (CD80), B7-2 (CD86), PD-L1, PD-L2, 4-1BBL, OX40L, inducible costimulatory ligand (ICOS-L), intercellular adhesion molecule (ICAM), CD30L, CD40, CD70, CD83, HLA-G, MICA, M1CB, HVEM, lymphotoxin beta receptor, 3/TR6, ILT3, ILT4, an agonist or antibody that binds Toll ligand receptor and a ligand that specifically binds with B7-H3.

A “costimulatory signal” refers to a signal, which in combination with a primary signal, such as TCR/CD3 ligation, leads to T cell proliferation and/or upregulation or downregulation of a molecule.

The term “extracellular ligand-binding domain,” as used herein, refers to an oligo- or polypeptide that is capable of binding a ligand, e.g., a cell surface molecule. For example, the extracellular ligand-binding domain can be chosen to recognize a ligand that acts as a cell surface marker on target cells associated with a particular disease state (e.g., cancer). Examples of cell surface markers that can act as ligands include those associated with viral, bacterial and parasitic infections, autoimmune disease and cancer cells.

A “signal transducing domain” or “signaling domain”, as used herein with respect to CARs, is responsible for intracellular signaling following the binding of an extracellular ligand binding domain to a target. In some aspects, the signaling domain as used in a CAR results in the activation of the immune cell and immune response. In other words, the signal transducing domain can responsible for the activation of at least one of the normal effector functions of the immune cell in which the CAR is expressed. For example, the effector function of a T cell can be a cytolytic activity or helper activity including the secretion of cytokines. Thus, the signal transducing domain can refer to the portion of a protein which transduces the effector function signal and directs the cell to perform a specialized function. Examples of signal transducing domains for use in a CAR can be the cytoplasmic sequences of the T cell receptor and co-receptors that act in concert to initiate signal transduction following antigen receptor engagement, as well as any derivate or variant of these sequences and any synthetic sequence that has the same functional capability. In some cases, signaling domains comprise two distinct classes of cytoplasmic signaling sequences, those that initiate antigen-dependent primary activation, and those that act in an antigen-independent manner to provide a secondary or co-stimulatory signal. Primary cytoplasmic signaling sequences can comprise signaling motifs which are known as immunoreceptor tyrosine-based activation motifs (ITAMs).

“ITAMs” refer to signaling motifs found in the intracytoplasmic tail of a variety of receptors that serve as binding sites for syk/zap70 class tyrosine kinases. Exemplary ITAMs include those derived from TCRzeta, FcRgamma, FcRbeta, FcRepsilon, CD3gamma, CD3delta, CD3epsilon, CD3zeta, CD5, CD22, CD79a, CD79b and CD66d. In some aspects, the signaling domain of the CAR can comprise the CD3zeta signaling domain.

The “transmembrane region” or “transmembrane domain” as used herein is a portion of a CAR that anchors the extracellular binding portion to the plasma membrane of an immune effector cell, and facilitates binding of the binding domain to the target antigen. In some aspects, the transmembrane domain can be a CD3zeta transmembrane domain, however other transmembrane domains that can be employed include those obtained from CD8α, CD4, CD28, CD45, CD9, CD16, CD22, CD33, CD64, CD80, CD86, CD134, CD137, or CD154.

The binding domain of a CAR can be followed by a “spacer,” or, “hinge,” which refers to the region that separates or moves the antigen binding domain away from the effector cell surface. In some aspects, the hinge enables cell/cell contact, antigen binding and activation (Patel et al., Gene Therapy, 1999; 6:412-419). The hinge region in a CAR can be between the transmembrane (TM) and the binding domain. In some aspects, a hinge region is an immunoglobulin hinge region and can be a wild type immunoglobulin hinge region or an altered wild type immunoglobulin hinge region. Other exemplary hinge regions used in the CARs disclosed herein can include the hinge region derived from the extracellular regions of type 1 membrane proteins such as CD8α, CD4, CD28 and CD7, which may be wild-type hinge regions from these molecules or may be altered. In some aspects, the hinge region comprises a CD8α hinge.

It is understood that wherever aspects are disclosed herein with the language “comprising,” otherwise analogous aspects disclosed in terms of “consisting of” and/or “consisting essentially of” are also provided.

The following examples are illustrative and do not limit the scope of the claimed aspects.

### II. Anti-CD94 Antibodies and Antigen-Binding Fragments Thereof

In some aspects, provided herein are antibodies (e.g. monoclonal antibodies) and antigen-binding fragments thereof which specifically bind to CD94 (e.g., human CD94). In some aspects, the amino acid sequence for human CD94 comprises SEQ ID NO: 31 (see RefSeq Accession Number NP_001107868.2).

In some aspects, an antibody or antigen-binding fragment thereof provided herein binds to CD94 and comprises six CDRs (e.g., a CDR H1, a CDR H2, a CDR H3, a CDR L1, a CDR L2, and a CDR L3). The CDRs can be determined by a number of algorithms in the art, such as IMGT (Table 1), abYsis (Table 2), and Kabat (Table 3).

For example, the international ImMunoGeneTics information System® (http://www.imgt.org) is based on the IMGT numbering for all immunoglobulin and T cell receptor V-REGIONs of all species (IMGT®, the international ImMunoGeneTics information System®; Lefranc M P et al., (1991) Nucleic Acids Res. 27 (1): 209-12; Ruiz M et al., (2000) Nucleic Acids Res. 28 (1): 219-21; Lefranc M P (2001) Nucleic Acids Res. 29 (1): 207-9; Lefranc M P (2003) Nucleic Acids Res. 31 (1): 307-10; Lefranc M P et al., (2005) Dev. Comp. Immunol. 29 (3): 185-203; Kaas Q et al., (2007) Briefings in Functional Genomics & Proteomics, 6 (4): 253-64).

The abYsis algorithm compiles antibody protein sequences from EMBLIG which contains antibody information extracted from the EMBL-ENA databank, the Kabat collection, and the Protein Databank (Swindells M B et al., (2017) J Mol Biol. 2017 Feb. 3; 429 (3): 356-364).

The Kabat definition is based on sequence variability and is the commonly used (Kabat E A et al., supra).

TABLE 1
IMGT CDR Amino Acid Sequences

| | | | SEQ ID |
|---|---|---|---|
| CDR | Algorithm | Sequence | NO |
| CDR H1 | IMGT | GFNIKDTY | 3 |
| CDR H2 | IMGT | IDPANGNT | 4 |
| CDR H3 | IMGT | VYGRGSAWFAF | 5 |
| CDR L1 | IMGT | QDISNY | 6 |
| CDR L2 | IMGT | YTS | — |
| CDR L3 | IMGT | QQGNTLPWT | 8 |

TABLE 2
abYsis CDR Amino Acid Sequences

| | | | SEQ ID |
|---|---|---|---|
| CDR | Algorithm | Sequence | NO |
| CDR H1 | ab Ysis | DTYMH | 19 |
| CDR H2 | ab Ysis | RIDPANGNTQYDPKFQG | 20 |
| CDR H3 | ab Ysis | GRGSAWFAF | 21 |
| CDR L1 | ab Ysis | RASQDISNYLN | 22 |
| CDR L2 | ab Ysis | YTSRLHS | 23 |
| CDR L3 | ab Ysis | QQGNTLPWT | 8 |

TABLE 3
Kabat CDR Amino Acid Sequences

| | | | SEQ ID |
|---|---|---|---|
| CDR | Algorithm | Sequence | NO |
| CDR H1 | Kabat | DTYMH | 19 |
| CDR H2 | Kabat | RIDPANGNTQYDPKFQG | 20 |
| CDR H3 | Kabat | GRGSAWFAF | 21 |
| CDR L1 | Kabat | RASQDISNYLN | 22 |
| CDR L2 | Kabat | YTSRLH | 24 |
| CDR L3 | Kabat | QQGNTLPWT | 8 |

In some aspects, provided herein is an antibody or antigen-binding fragment thereof capable of binding to CD94 (e.g., an anti-CD94 specific antibody), wherein the antibody or antigen-binding fragment thereof comprises a complementary determining region (CDR) H1 comprising the amino acid sequence set forth in SEQ ID NO: 3 or SEQ ID NO: 19, a CDR H2 comprising the amino acid sequence set forth in SEQ ID NO: 4 or SEQ ID NO: 20, a CDR H3 comprising the amino acid sequence set forth in SEQ ID NO: 5 or SEQ ID NO: 21, a CDR L1 comprising the amino acid sequence set forth in SEQ ID NO: 6 or SEQ ID NO: 22, a CDR L2 comprising the amino acid sequence YTS or the amino acid sequence set forth in SEQ ID NO: 23 or SEQ ID NO: 24, and a CDR L3 comprising the amino acid sequence set forth in SEQ ID NO: 8.

In some aspects, the antibody or antigen-binding fragment thereof capable of binding CD94 (e.g., an anti-CD94 specific antibody) comprises a complementary determining region (CDR) H1 comprising the amino acid sequence set forth in SEQ ID NO: 3, a CDR H2 comprising the amino acid sequence set forth in SEQ ID NO: 4, a CDR H3 comprising the amino acid sequence set forth in SEQ ID NO: 5, a CDR L1 comprising the amino acid sequence set forth in SEQ ID NO: 6, a CDR L2 comprising the amino acid sequence YTS, and a CDR L3 comprising the amino acid sequence set forth in SEQ ID NO: 8.

In some aspects, the antibody or antigen-binding fragment thereof capable of binding CD94 (e.g., an anti-CD94 specific antibody) comprises a complementary determining region (CDR) H1 comprising the amino acid sequence set forth in SEQ ID NO: 19, a CDR H2 comprising the amino acid sequence set forth in SEQ ID NO: 20, a CDR H3 comprising the amino acid sequence set forth in SEQ ID NO: 21, a CDR L1 comprising the amino acid sequence set forth in SEQ ID NO: 22, a CDR L2 comprising the amino acid sequence set forth in SEQ ID NO: 23, and a CDR L3 comprising the amino acid sequence set forth in SEQ ID NO: 8.

In some aspects, the antibody or antigen-binding fragment thereof capable of binding CD94 (e.g., an anti-CD94 specific antibody) comprises a complementary determining region (CDR) H1 comprising the amino acid sequence set forth in SEQ ID NO: 19, a CDR H2 comprising the amino acid sequence set forth in SEQ ID NO: 20, a CDR H3 comprising the amino acid sequence set forth in SEQ ID NO: 21, a CDR L1 comprising the amino acid sequence set forth in SEQ ID NO: 22, a CDR L2 comprising the amino acid sequence set forth in SEQ ID NO: 24, and a CDR L3 comprising the amino acid sequence set forth in SEQ ID NO: 8.

In some aspects, the antibody or antigen-binding fragment thereof capable of binding CD94 (e.g., an anti-CD94 specific antibody) comprises a heavy chain variable region (VH) and a light chain variable region (VL). In some aspects, the VH comprises an amino acid sequence having at least 85%, at least 90%, at least 91% at least 92%, at least 93%, at least 94%, at least 95%, at least 96%, at least 97%, at least 98%, at least 99%, or 100% sequence identity to SEQ ID NO: 1. In some aspects, the VL comprises an amino acid sequence having at least 85%, at least 90%, at least 91%, at least 92%, at least 93%, at least 94%, at least 95%, at least 96%, at least 97%, at least 98%, at least 99%, or 100% sequence identity to SEQ ID NO: 2.

In some aspects, the antibody or antigen-binding fragment thereof capable of binding CD94 (e.g., an anti-CD94 specific antibody) comprises a heavy chain variable region (VH) and a light chain variable region (VL). In some aspects, the VH comprises an amino acid sequence having about 85%, about 90%, about 91% about 92%, about 93%, about 94%, about 95%, about 96%, about 97%, about 98%, about 99%, or about 100% sequence identity to SEQ ID NO: 1. In some aspects, the VL comprises an amino acid sequence having about 85%, about 90%, about 91%, about 92%, about 93%, about 94%, about 95%, about 96%, about 97%, about 98%, about 99%, or about 100% sequence identity to SEQ ID NO: 2.

In some aspects, the antibody or antigen-binding fragment comprises a VH comprising an amino acid sequence at least 85% identical to the amino acid sequence SEQ ID NO: 1. In some aspects, the antibody or antigen-binding fragment comprises a VH comprising an amino acid sequence at least 90% identical to the amino acid sequence SEQ ID NO: 1. In some aspects, the antibody or antigen-binding fragment comprises a VH comprising an amino acid sequence at least 95% identical to the amino acid sequence SEQ ID NO: 1. In some aspects, the antibody or antigen-binding fragment comprises a VH comprising an amino acid sequence at least 98% identical to the amino acid sequence SEQ ID NO: 1. In some aspects, the antibody or antigen-binding fragment comprises a VH comprising an amino acid sequence at least 99% identical to the amino acid sequence SEQ ID NO: 1. In some aspects, the antibody or antigen-binding fragment comprises a VH comprising an amino acid sequence corresponding to the amino acid sequence of SEQ ID NO: 1.

In some aspects, the antibody or antigen-binding fragment comprises a VL comprising an amino acid sequence at least 85% identical to the amino acid sequence of SEQ ID NO: 2. In some aspects, the antibody or antigen-binding fragment comprises a VL comprising an amino acid sequence at least 90% identical to the amino acid sequence of SEQ ID NO: 2. In some aspects, the antibody or antigen-binding fragment comprises a VL comprising an amino acid sequence at least 95% identical to the amino acid sequence of SEQ ID NO: 2. In some aspects, the antibody or antigen-binding fragment comprises a VL comprising an amino acid sequence at least 98% identical to the amino acid sequence of SEQ ID NO: 2. In some aspects, the antibody or antigen-binding fragment comprises a VL comprising an amino acid sequence at least 99% identical to the amino acid sequence of SEQ ID NO: 2. In some aspects, the antibody or antigen-binding fragment comprises a VL comprising an amino acid sequence corresponding to the amino acid sequence of SEQ ID NO: 2.

In some aspects, the antibody or antigen-binding fragment comprises a VH comprising an amino acid sequence about 85% identical to the amino acid sequence SEQ ID NO: 1. In some aspects, the antibody or antigen-binding fragment comprises a VH comprising an amino acid sequence about 90% identical to the amino acid sequence SEQ ID NO: 1. In some aspects, the antibody or antigen-binding fragment comprises a VH comprising an amino acid sequence about 95% identical to the amino acid sequence SEQ ID NO: 1. In some aspects, the antibody or antigen-binding fragment comprises a VH comprising an amino acid sequence about 98% identical to the amino acid sequence SEQ ID NO: 1. In some aspects, the antibody or antigen-binding fragment comprises a VH comprising an amino acid sequence about 99% identical to the amino acid sequence SEQ ID NO: 1.

In some aspects, the antibody or antigen-binding fragment comprises a VL comprising an amino acid sequence about 85% identical to the amino acid sequence of SEQ ID NO: 2. In some aspects, the antibody or antigen-binding fragment comprises a VL comprising an amino acid sequence about 90% identical to the amino acid sequence of SEQ ID NO: 2. In some aspects, the antibody or antigen-binding fragment comprises a VL comprising an amino acid sequence about 95% identical to the amino acid sequence of SEQ ID NO: 2. In some aspects, the antibody or antigen-binding fragment comprises a VL comprising an amino acid sequence about 98% identical to the amino acid sequence of SEQ ID NO: 2. In some aspects, the antibody or antigen-binding fragment comprises a VL comprising an amino acid sequence about 99% identical to the amino acid sequence of SEQ ID NO: 2.

In some aspects, provided herein is an antibody or antigen-binding fragment thereof capable of binding to CD94 (e.g., an anti-CD94 specific antibody), wherein the antibody or antigen-binding fragment thereof comprises: i) a CDR H1, CDR H2, and CDR H3 comprising the CDR H1, CDR H2, and CDR H3 amino acid sequences of SEQ ID NO: 1; and ii) a CDR L1, CDR L2, and CDR L3 comprising the CDR L1, CDR L2, and CDR L3 amino acid sequences of SEQ ID NO: 2.

In some aspects, the CDRs are the Kabat-defined CDRs, the Chothia-defined CDRs, the AbM-defined CDRs, or the IMGT-defined CDRs.

In some aspects, the antibody or antigen-binding fragment thereof is human, humanized, or chimeric.

In some aspects, the antibody or antigen-binding fragment thereof is an IgG antibody.

In some aspects, the IgG antibody is an IgG1 antibody or an IgG4 antibody.

In some aspects, the antibody is an antigen-binding fragment of an antibody.

In some aspects, said fragment is selected from the group consisting of Fab, F(ab′)2, Fv, scFv, scFv-Fc, dsFv and a single domain molecule.

In some aspects, said fragment is a scFv.

In some aspects, said fragment is a Fab.

In some aspects, said fragment is an intrabody.

In some aspects, the antigen-binding fragment is devoid of an Fc region.

In some aspects, the antibody or antigen-binding fragment thereof comprises a VH and a VL on the same polypeptide chain.

In some aspects, the VH and VL are connected by a linker.

In some aspects, the antibody or antigen-binding fragment thereof is conjugated to an agent selected from the group consisting of a therapeutic agent, a prodrug, a peptide, a protein, an enzyme, a virus, a lipid, a biological response modifier, a pharmaceutical agent, and PEG.

In some aspects, the antibody or antigen-binding fragment thereof is a bispecific antibody. The antibody can be in the form of scFv, Fab, F (ab) 2, or IgG conjugated with anti-CD3 antibody, anti-CD16 antibody, or other molecules, to mediate effector response via T, NK, or macrophage immune cells against CD94-expressing target cells.

In some aspects, an antibody or antigen-binding fragment thereof disclosed herein can be described by its VL domain alone or its VH domain alone. See, for example, Rader C et al., (1998) PNAS 95:8910-8915, which is incorporated herein by reference in its entirety, describing the humanization of the mouse anti-αvβ3 antibody by identifying a complementing light chain or heavy chain, respectively, from a human light chain or heavy chain library, resulting in humanized antibody variants having affinities as high or higher than the affinity of the original antibody. See also Clackson T et al., (1991) Nature 352:624-628, which is incorporated herein by reference in its entirety, describing methods of producing antibodies that bind a specific antigen by using a specific VL domain (or VH domain) and screening a library for the complementary variable domains. See also Kim S J & Hong H J, (2007) J Microbiol 45:572-577, which is incorporated herein by reference in its entirety, describing methods of producing antibodies that bind a specific antigen by using a specific VH domain and screening a library (e.g., human VL library) for complementary VL domains; the selected VL domains in turn could be used to guide selection of additional complementary (e.g., human) VH domains.

In some aspects, the CDRs of an antibody or antigen-binding fragment thereof can be determined according to the Chothia numbering scheme, which refers to the location of immunoglobulin structural loops (see, e.g., Chothia C & Lesk A M, (1987), J Mol Biol 196:901-917; Al-Lazikani B et al., (1997) J Mol Biol 273:927-948; Chothia C et al., (1992) J Mol Biol 227:799-817; Tramontano A et al., (1990) J Mol Biol 215(1): 175-82; and U.S. Pat. No. 7,709,226). Typically, when using the Kabat numbering convention, the Chothia CDR-H1 loop is present at heavy chain amino acids 26 to 32, 33, or 34, the Chothia CDR-H2 loop is present at heavy chain amino acids 52 to 56, and the Chothia CDR-H3 loop is present at heavy chain amino acids 95 to 102, while the Chothia CDR-L1 loop is present at light chain amino acids 24 to 34, the Chothia CDR-L2 loop is present at light chain amino acids 50 to 56, and the Chothia CDR-L3 loop is present at light chain amino acids 89 to 97. The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34).

In some aspects, provided herein are antibodies and antigen-binding fragments thereof that specifically bind to CD94 (e.g., human CD94) and comprise the Chothia V H and VL CDRs of an antibody. In some aspects, antibodies or antigen-binding fragments thereof that specifically bind to CD94 (e.g., human CD94) comprise one or more CDRs, in which the Chothia and Kabat CDRs have the same amino acid sequence. In some aspects, provided herein are antibodies and antigen-binding fragments thereof that specifically bind to CD94 (e.g., human CD94) and comprise combinations of Kabat CDRs and Chothia CDRs.

In some aspects, the CDRs of an antibody or antigen-binding fragment thereof can be determined according to the IMGT numbering system as described in Lefranc M P, (1999) The Immunologist 7:132-136 and Lefranc M P et al., (1999) Nucleic Acids Res 27:209-212. According to the IMGT numbering scheme, CDR H1 is at positions 26 to 35, CDR H2 is at positions 51 to 57, CDR H3 is at positions 93 to 102, CDR L1 is at positions 27 to 32, CDR L2 is at positions 50 to 52, and CDR L3 is at positions 89 to 97. In some aspects, provided herein are antibodies and antigen-binding fragments thereof that specifically bind to CD94 (e.g., human CD94) and comprise the IMGT VH and VL CDRs of an antibody listed in Tables 3 and 4, for example, as described in Lefranc M P (1999) supra and Lefranc M P et al., (1999) supra).

In some aspects, the CDRs of an antibody or antigen-binding fragment thereof can be determined according to MacCallum R M et al., (1996) J Mol Biol 262:732-745. See also, e.g., Martin A. “Protein Sequence and Structure Analysis of Antibody Variable Domains,” in Antibody Engineering, Kontermann and Dübel, eds., Chapter 31, pp. 422-439, Springer-Verlag, Berlin (2001). In some aspects, provided herein are antibodies or antigen-binding fragments thereof that specifically bind to CD94 (e.g., human CD94) are determined by the method in MacCallum R M et al.

In some aspects, the CDRs of an antibody or antigen-binding fragment thereof can be determined according to the AbM numbering scheme, which refers AbM hypervariable regions which represent a compromise between the Kabat CDRs and Chothia structural loops, and are used by Oxford Molecular's AbM antibody modeling software (Oxford Molecular Group, Inc.). In some aspects, provided herein are antibodies or antigen-binding fragments thereof that specifically bind to CD94 (e.g., human CD94) are determined by the AbM numbering scheme.

In some aspects, an antibody or antigen-binding fragment thereof disclosed herein is an isolated antibody or antigen-binding fragment thereof. In some aspects, an antibody or antigen-binding fragment thereof disclosed herein is a monoclonal antibody or antigen-binding fragment thereof. In some aspects, an antibody or antigen-binding fragment thereof disclosed herein is not a polyclonal antibody or antigen-binding fragment thereof.

In some aspects, an antigen-binding fragment as disclosed herein, which immunospecifically binds to CD94 (e.g., human CD94), is selected from the group consisting of a Fab, Fab′, F(ab′)2, and scFv, wherein the Fab, Fab′, F(ab′)2, or scFv comprises a heavy chain variable region sequence and a light chain variable region sequence of an anti-CD94 antibody or antigen-binding fragment thereof as disclosed herein. A Fab, Fab′, F(ab′)2, or scFv can be produced by any technique known to those of skill in the art. In some aspects, the Fab, Fab′, F(ab′)2, or scFv further comprises a moiety that extends the half-life of the antibody in vivo. The moiety is also termed a “half-life extending moiety.” Any moiety known to those of skill in the art for extending the half-life of a Fab, Fab′, F(ab′)2, or scFv in vivo can be used. For example, the half-life extending moiety can include a Fc region, a polymer, an albumin, or an albumin binding protein or compound. The polymer can include a natural or synthetic, optionally substituted straight or branched chain polyalkylene, polyalkenylene, polyoxyalkylene, polysaccharide, polyethylene glycol, polypropylene glycol, polyvinyl alcohol, methoxypolyethylene glycol, lactose, amylose, dextran, glycogen, or derivative thereof. Substituents can include one or more hydroxy, methyl, or methoxy groups. In some aspects, the Fab, Fab′, F(ab′)2, or scFv can be modified by the addition of one or more C-terminal amino acids for attachment of the half-life extending moiety. In some aspects, the half-life extending moiety is polyethylene glycol or human serum albumin. In some aspects, the Fab, Fab′, F(ab′)2, or scFv is fused to an Fc region.

In some aspects, an anti-CD94 antibody or antigen-binding fragment thereof disclosed herein is a bispecific antibody or antigen-binding fragment thereof.

An anti-CD94 antibody or antigen-binding fragment thereof can be fused or conjugated (e.g., covalently or noncovalently linked) to a detectable label or substance. Examples of detectable labels or substances include enzyme labels, such as, glucose oxidase; radioisotopes, such as iodine (¹²⁵I, ¹²¹I), carbon (¹⁴C), sulfur (³⁵S), tritium (³H), indium (¹²¹In), and technetium (⁹⁹Tc); luminescent labels, such as luminol; and fluorescent labels, such as fluorescein and rhodamine, and biotin. Such labeled antibodies or antigen-binding fragments thereof can be used to detect CD94 (e.g., human CD94) protein.

### III. Anti-CD94 Chimeric Antigen Receptors

Chimeric antigen receptors (CARs) redirect T cell specificity toward antibody-recognized antigens expressed on the surface of cells (e.g., cancer cells).

In some aspects, the present disclosure includes a chimeric antigen receptor (CAR) which is specific for CD94. In some aspects a CAR as disclosed herein comprises an extracellular target-specific binding domain, a transmembrane domain, an intracellular signaling domain (such as a signaling domain derived from CD3zeta or FcRgamma), and/or one or more co-stimulatory signaling domains derived from a co-stimulatory molecule, such as, but not limited to, 4-1BB, CD28, or OX40. In some aspects, the CAR includes a hinge or spacer region between the extracellular binding domain and the transmembrane domain, such as a CD8α hinge.

In some aspects, provided herein is a chimeric antigen receptor which comprises, from N-terminus to C-terminus: (a) an extracellular ligand-binding domain comprising an antigen-binding domain capable of binding to CD94; (b) a hinge; (c) a transmembrane domain; and (d) a cytoplasmic domain comprising a costimulatory domain and a signaling domain. In some aspects, the antigen-binding domain comprises a CDR H1 comprising the amino acid sequence set forth in SEQ ID NO: 3 or SEQ ID NO: 19, a CDR H2 comprising the amino acid sequence set forth in SEQ ID NO: 4 or SEQ ID NO: 20, a CDR H3 comprising the amino acid sequence set forth in SEQ ID NO: 5 or SEQ ID NO: 21, a CDR L1 comprising the amino acid sequence set forth in SEQ ID NO: 6 or SEQ ID NO: 22, a CDR L2 comprising the amino acid sequence YTS or the amino acid sequence set forth in SEQ ID NO: 23 or SEQ ID NO: 24, and a CDR L3 comprising the amino acid sequence set forth in SEQ ID NO: 8.

In some aspects, the antigen-binding domain comprises a CDR H1 comprising the amino acid sequence set forth in SEQ ID NO: 3, a CDR H2 comprising the amino acid sequence set forth in SEQ ID NO: 4, a CDR H3 comprising the amino acid sequence set forth in SEQ ID NO: 5, a CDR L1 comprising the amino acid sequence set forth in SEQ ID NO: 6, a CDR L2 comprising the amino acid sequence YTS, and a CDR L3 comprising the amino acid sequence set forth in SEQ ID NO: 8.

In some aspects, the antigen-binding domain comprises a CDR H1 comprising the amino acid sequence set forth in SEQ ID NO: 19, a CDR H2 comprising the amino acid sequence set forth in SEQ ID NO: 20, a CDR H3 comprising the amino acid sequence set forth in SEQ ID NO: 21, a CDR L1 comprising the amino acid sequence set forth in SEQ ID NO: 22, a CDR L2 comprising the amino acid sequence set forth in SEQ ID NO: 23, and a CDR L3 comprising the amino acid sequence set forth in SEQ ID NO: 8.

In some aspects, the antigen-binding domain comprises a CDR H1 comprising the amino acid sequence set forth in SEQ ID NO: 19, a CDR H2 comprising the amino acid sequence set forth in SEQ ID NO: 20, a CDR H3 comprising the amino acid sequence set forth in SEQ ID NO: 21, a CDR L1 comprising the amino acid sequence set forth in SEQ ID NO: 22, a CDR L2 comprising the amino acid sequence set forth in SEQ ID NO: 24, and a CDR L3 comprising the amino acid sequence set forth in SEQ ID NO: 8.

In some aspects, the chimeric antigen receptor comprises an amino acid sequence having at least 85%, at least 90%, at least 91% at least 92%, at least 93%, at least 94%, at least 95%, at least 96%, at least 97%, at least 98%, at least 99%, or 100% sequence identity to SEQ ID NO: 9.

In some aspects, the chimeric antigen receptor comprises an amino acid sequence having about 85%, about 90%, about 91% about 92%, about 93%, about 94%, about 95%, about 96%, about 97%, about 98%, about 99%, or about 100% sequence identity to SEQ ID NO: 9.

In some aspects, the chimeric antigen receptor comprises an amino acid sequence having at least 85%, at least 90%, at least 91% at least 92%, at least 93%, at least 94%, at least 95%, at least 96%, at least 97%, at least 98%, at least 99%, or 100% sequence identity to SEQ ID NO: 56.

In some aspects, the chimeric antigen receptor comprises an amino acid sequence having about 85%, about 90%, about 91% about 92%, about 93%, about 94%, about 95%, about 96%, about 97%, about 98%, about 99%, or about 100% sequence identity to SEQ ID NO: 56.

In some aspects, the chimeric antigen receptor comprises an amino acid sequence having at least 85%, at least 90%, at least 91% at least 92%, at least 93%, at least 94%, at least 95%, at least 96%, at least 97%, at least 98%, at least 99%, or 100% sequence identity to SEQ ID NO: 57.

In some aspects, the chimeric antigen receptor comprises an amino acid sequence having about 85%, about 90%, about 91% about 92%, about 93%, about 94%, about 95%, about 96%, about 97%, about 98%, about 99%, or about 100% sequence identity to SEQ ID NO: 57.

In some aspects, the chimeric antigen receptor comprises a heavy chain variable region (VH) comprising the amino acid sequence of SEQ ID NO: 1 and a light chain variable region (VL) comprising the amino acid sequence of SEQ ID NO: 2.

In some aspects, the VH comprises an amino acid sequence having at least 85%, at least 90%, at least 91% at least 92%, at least 93%, at least 94%, at least 95%, at least 96%, at least 97%, at least 98%, at least 99%, or 100% sequence identity to SEQ ID NO: 1. In some aspects, the VL comprises an amino acid sequence having at least 85%, at least 90%, at least 91%, at least 92%, at least 93%, at least 94%, at least 95%, at least 96%, at least 97%, at least 98%, at least 99%, or 100% sequence identity to SEQ ID NO: 2.

In some aspects, the VH comprises an amino acid sequence having about 85%, about 90%, about 91% about 92%, about 93%, about 94%, about 95%, about 96%, about 97%, about 98%, about 99%, or about 100% sequence identity to SEQ ID NO: 1. In some aspects, the VL comprises an amino acid sequence having about 85%, about 90%, about 91%, about 92%, about 93%, about 94%, about 95%, about 96%, about 97%, about 98%, about 99%, or about 100% sequence identity to SEQ ID NO: 2.

In some aspects, the VH comprises an amino acid sequence at least 85% identical to the amino acid sequence SEQ ID NO: 1. In some aspects, the VH comprises an amino acid sequence at least 90% identical to the amino acid sequence SEQ ID NO: 1. In some aspects, the VH comprises an amino acid sequence at least 95% identical to the amino acid sequence SEQ ID NO: 1. In some aspects, the VH comprises an amino acid sequence at least 98% identical to the amino acid sequence SEQ ID NO: 1. In some aspects, the VH comprises an amino acid sequence at least 99% identical to the amino acid sequence SEQ ID NO: 1. In some aspects, the VH comprises an amino acid sequence corresponding to the amino acid sequence of SEQ ID NO: 1.

In some aspects, the VL comprises an amino acid sequence at least 85% identical to the amino acid sequence of SEQ ID NO: 2. In some aspects, the VL comprises an amino acid sequence at least 90% identical to the amino acid sequence of SEQ ID NO: 2. In some aspects, the VL comprises an amino acid sequence at least 95% identical to the amino acid sequence of SEQ ID NO: 2. In some aspects, the VL comprises an amino acid sequence at least 98% identical to the amino acid sequence of SEQ ID NO: 2. In some aspects, the VL comprises an amino acid sequence at least 99% identical to the amino acid sequence of SEQ ID NO: 2. In some aspects, the VL comprises an amino acid sequence corresponding to the amino acid sequence of SEQ ID NO: 2.

In some aspects, the VH comprises an amino acid sequence about 85% identical to the amino acid sequence SEQ ID NO: 1. In some aspects, the VH comprises an amino acid sequence about 90% identical to the amino acid sequence SEQ ID NO: 1. In some aspects, the VH comprises an amino acid sequence about 95% identical to the amino acid sequence SEQ ID NO: 1. In some aspects, the VH comprises an amino acid sequence about 98% identical to the amino acid sequence SEQ ID NO: 1. In some aspects, the VH comprises an amino acid sequence about 99% identical to the amino acid sequence SEQ ID NO: 1.

In some aspects, the VL comprises an amino acid sequence about 85% identical to the amino acid sequence of SEQ ID NO: 2. In some aspects, the VL comprises an amino acid sequence about 90% identical to the amino acid sequence of SEQ ID NO: 2. In some aspects, the VL comprises an amino acid sequence about 95% identical to the amino acid sequence of SEQ ID NO: 2. In some aspects, the VL comprises an amino acid sequence about 98% identical to the amino acid sequence of SEQ ID NO: 2. In some aspects, the VL comprises an amino acid sequence about 99% identical to the amino acid sequence of SEQ ID NO: 2.

In some aspects, the antigen-binding domain is a scFv. In some aspects, the scFv comprises an amino acid sequence having at least 85%, at least 90%, at least 91% at least 92%, at least 93%, at least 94%, at least 95%, at least 96%, at least 97%, at least 98%, at least 99%, or 100% sequence identity to SEQ ID NO: 33. In some aspects, the scFv comprises an amino acid sequence having at least about 85%, at least about 90%, at least about 91%, at least about 92%, at least about 93%, at least about 94%, at least about 95%, at least about 96%, at least about 97%, at least about 98%, at least about 99%, or 100% sequence identity to SEQ ID NO: 33. In some aspects, the scFv comprises an amino acid sequence having about 85%, about 90%, about 91%, about 92%, about 93%, about 94%, about 95%, about 96%, about 97%, about 98%, about 99%, or about 100% sequence identity to SEQ ID NO: 33.

In some aspects, the scFv comprises an amino acid sequence at least 85% identical to the amino acid sequence of SEQ ID NO: 33. In some aspects, the scFv comprises an amino acid sequence at least 90% identical to the amino acid sequence of SEQ ID NO: 33. In some aspects, the scFv comprises an amino acid sequence at least 95% identical to the amino acid sequence of SEQ ID NO: 33. In some aspects, the scFv comprises an amino acid sequence at least 98% identical to the amino acid sequence of SEQ ID NO: 33. In some aspects, the scFv comprises an amino acid sequence at least 99% identical to the amino acid sequence of SEQ ID NO: 33. In some aspects, the scFv comprises an amino acid sequence corresponding to the amino acid sequence of SEQ ID NO: 33. In some aspects, the scFv comprises an amino acid sequence about 85% identical to the amino acid sequence SEQ ID NO: 33. In some aspects, the scFv comprises an amino acid sequence about 90% identical to the amino acid sequence SEQ ID NO: 33. In some aspects, the scFv comprises an amino acid sequence about 95% identical to the amino acid sequence SEQ ID NO: 33. In some aspects, the scFv comprises an amino acid sequence about 98% identical to the amino acid sequence SEQ ID NO: 33. In some aspects, the scFv comprises an amino acid sequence about 99% identical to the amino acid sequence SEQ ID NO: 33.

In some aspects, the costimulatory domain comprises a 4-1BB costimulatory domain. In some aspects, the 4-1BB costimulatory domain comprises an amino acid sequence having at least 85%, at least 90%, at least 91% at least 92%, at least 93%, at least 94%, at least 95%, at least 96%, at least 97%, at least 98%, at least 99%, or 100% sequence identity to SEQ ID NO: 37. In some aspects, the 4-1BB costimulatory domain comprises an amino acid sequence having at least about 85%, at least about 90%, at least about 91%, at least about 92%, at least about 93%, at least about 94%, at least about 95%, at least about 96%, at least about 97%, at least about 98%, at least about 99%, or 100% sequence identity to SEQ ID NO: 37. In some aspects, the 4-1BB costimulatory domain comprises an amino acid sequence having about 85%, about 90%, about 91%, about 92%, about 93%, about 94%, about 95%, about 96%, about 97%, about 98%, about 99%, or about 100% sequence identity to SEQ ID NO: 37.

In some aspects, the 4-1BB costimulatory domain comprises an amino acid sequence at least 85% identical to the amino acid sequence of SEQ ID NO: 37. In some aspects, the 4-1BB costimulatory domain comprises an amino acid sequence at least 90% identical to the amino acid sequence of SEQ ID NO: 37. In some aspects, the 4-1BB costimulatory domain comprises an amino acid sequence at least 95% identical to the amino acid sequence of SEQ ID NO: 37. In some aspects, the 4-1BB costimulatory domain comprises an amino acid sequence at least 98% identical to the amino acid sequence of SEQ ID NO: 37. In some aspects, the 4-1BB costimulatory domain comprises an amino acid sequence at least 99% identical to the amino acid sequence of SEQ ID NO: 37. In some aspects, the 4-1BB costimulatory domain comprises an amino acid sequence corresponding to the amino acid sequence of SEQ ID NO: 37. In some aspects, the 4-1BB costimulatory domain comprises an amino acid sequence about 85% identical to the amino acid sequence SEQ ID NO: 37. In some aspects, the 4-1BB costimulatory domain comprises an amino acid sequence about 90% identical to the amino acid sequence SEQ ID NO: 37. In some aspects, the 4-1BB costimulatory domain comprises an amino acid sequence about 95% identical to the amino acid sequence SEQ ID NO: 37. In some aspects, the 4-1BB costimulatory domain comprises an amino acid sequence about 98% identical to the amino acid sequence SEQ ID NO: 37. In some aspects, the 4-1BB costimulatory domain comprises an amino acid sequence about 99% identical to the amino acid sequence SEQ ID NO: 37.

In some aspects, the costimulatory domain comprises a CD28 costimulatory domain. In some aspects, the CD28 costimulatory domain comprises an amino acid sequence having at least 85%, at least 90%, at least 91% at least 92%, at least 93%, at least 94%, at least 95%, at least 96%, at least 97%, at least 98%, at least 99%, or 100% sequence identity to SEQ ID NO: 52. In some aspects, the CD28 costimulatory domain comprises an amino acid sequence having at least about 85%, at least about 90%, at least about 91%, at least about 92%, at least about 93%, at least about 94%, at least about 95%, at least about 96%, at least about 97%, at least about 98%, at least about 99%, or 100% sequence identity to SEQ ID NO: 52. In some aspects, the CD28 costimulatory domain comprises an amino acid sequence having about 85%, about 90%, about 91%, about 92%, about 93%, about 94%, about 95%, about 96%, about 97%, about 98%, about 99%, or about 100% sequence identity to SEQ ID NO: 52.

In some aspects, the CD28 costimulatory domain comprises an amino acid sequence at least 85% identical to the amino acid sequence of SEQ ID NO: 52. In some aspects, the CD28 costimulatory domain comprises an amino acid sequence at least 90% identical to the amino acid sequence of SEQ ID NO: 52. In some aspects, the CD28 costimulatory domain comprises an amino acid sequence at least 95% identical to the amino acid sequence of SEQ ID NO: 52. In some aspects, the CD28 costimulatory domain comprises an amino acid sequence at least 98% identical to the amino acid sequence of SEQ ID NO: 52. In some aspects, the CD28 costimulatory domain comprises an amino acid sequence at least 99% identical to the amino acid sequence of SEQ ID NO: 52. In some aspects, the CD28 costimulatory domain comprises an amino acid sequence corresponding to the amino acid sequence of SEQ ID NO: 52. In some aspects, the CD28 costimulatory domain comprises an amino acid sequence about 85% identical to the amino acid sequence SEQ ID NO: 52. In some aspects, the CD28 costimulatory domain comprises an amino acid sequence about 90% identical to the amino acid sequence SEQ ID NO: 52. In some aspects, the CD28 costimulatory domain comprises an amino acid sequence about 95% identical to the amino acid sequence SEQ ID NO: 52. In some aspects, the CD28 costimulatory domain comprises an amino acid sequence about 98% identical to the amino acid sequence SEQ ID NO: 52. In some aspects, the CD28 costimulatory domain comprises an amino acid sequence about 99% identical to the amino acid sequence SEQ ID NO: 52.

In some aspects, the costimulatory domain comprises an OX40 costimulatory domain. In some aspects, the OX40 costimulatory domain comprises an amino acid sequence having at least 85%, at least 90%, at least 91% at least 92%, at least 93%, at least 94%, at least 95%, at least 96%, at least 97%, at least 98%, at least 99%, or 100% sequence identity to SEQ ID NO: 53. In some aspects, the OX40 costimulatory domain comprises an amino acid sequence having at least about 85%, at least about 90%, at least about 91%, at least about 92%, at least about 93%, at least about 94%, at least about 95%, at least about 96%, at least about 97%, at least about 98%, at least about 99%, or 100% sequence identity to SEQ ID NO: 53. In some aspects, the OX40 costimulatory domain comprises an amino acid sequence having about 85%, about 90%, about 91%, about 92%, about 93%, about 94%, about 95%, about 96%, about 97%, about 98%, about 99%, or about 100% sequence identity to SEQ ID NO: 53.

In some aspects, the OX40 costimulatory domain comprises an amino acid sequence at least 85% identical to the amino acid sequence of SEQ ID NO: 53. In some aspects, the OX40 costimulatory domain comprises an amino acid sequence at least 90% identical to the amino acid sequence of SEQ ID NO: 53. In some aspects, the OX40 costimulatory domain comprises an amino acid sequence at least 95% identical to the amino acid sequence of SEQ ID NO: 53. In some aspects, the OX40 costimulatory domain comprises an amino acid sequence at least 98% identical to the amino acid sequence of SEQ ID NO: 53. In some aspects, the OX40 costimulatory domain comprises an amino acid sequence at least 99% identical to the amino acid sequence of SEQ ID NO: 53. In some aspects, the OX40 costimulatory domain comprises an amino acid sequence corresponding to the amino acid sequence of SEQ ID NO: 53. In some aspects, the OX40 costimulatory domain comprises an amino acid sequence about 85% identical to the amino acid sequence SEQ ID NO: 53. In some aspects, the OX40 costimulatory domain comprises an amino acid sequence about 90% identical to the amino acid sequence SEQ ID NO: 53. In some aspects, the OX40 costimulatory domain comprises an amino acid sequence about 95% identical to the amino acid sequence SEQ ID NO: 53. In some aspects, the OX40 costimulatory domain comprises an amino acid sequence about 98% identical to the amino acid sequence SEQ ID NO: 53. In some aspects, the OX40 costimulatory domain comprises an amino acid sequence about 99% identical to the amino acid sequence SEQ ID NO: 53.

In some aspects, the hinge, the transmembrane domain, or both, are from a CD8α polypeptide.

In some aspects, the CD8α hinge domain comprises an amino acid sequence having at least 85%, at least 90%, at least 91% at least 92%, at least 93%, at least 94%, at least 95%, at least 96%, at least 97%, at least 98%, at least 99%, or 100% sequence identity to SEQ ID NO: 35. In some aspects, the CD8α hinge domain comprises an amino acid sequence having at least about 85%, at least about 90%, at least about 91%, at least about 92%, at least about 93%, at least about 94%, at least about 95%, at least about 96%, at least about 97%, at least about 98%, at least about 99%, or 100% sequence identity to SEQ ID NO: 35. In some aspects, the CD8α hinge domain comprises an amino acid sequence having about 85%, about 90%, about 91%, about 92%, about 93%, about 94%, about 95%, about 96%, about 97%, about 98%, about 99%, or about 100% sequence identity to SEQ ID NO: 35.

In some aspects, the CD8α hinge domain comprises an amino acid sequence at least 85% identical to the amino acid sequence of SEQ ID NO: 35. In some aspects, the CD8α hinge domain comprises an amino acid sequence at least 90% identical to the amino acid sequence of SEQ ID NO: 35. In some aspects, the CD8α hinge domain comprises an amino acid sequence at least 95% identical to the amino acid sequence of SEQ ID NO: 35. In some aspects, the CD8α hinge domain comprises an amino acid sequence at least 98% identical to the amino acid sequence of SEQ ID NO: 35. In some aspects, the CD8α hinge domain comprises an amino acid sequence at least 99% identical to the amino acid sequence of SEQ ID NO: 35. In some aspects, the CD8α hinge domain comprises an amino acid sequence corresponding to the amino acid sequence of SEQ ID NO: 35. In some aspects, the CD8α hinge domain comprises an amino acid sequence about 85% identical to the amino acid sequence SEQ ID NO: 35. In some aspects, the CD8α hinge domain comprises an amino acid sequence about 90% identical to the amino acid sequence SEQ ID NO: 35. In some aspects, the CD8α hinge domain comprises an amino acid sequence about 95% identical to the amino acid sequence SEQ ID NO: 35. In some aspects, the CD8α hinge domain comprises an amino acid sequence about 98% identical to the amino acid sequence SEQ ID NO: 35. In some aspects, the CD8α hinge domain comprises an amino acid sequence about 99% identical to the amino acid sequence SEQ ID NO: 35.

In some aspects, the CD8α transmembrane domain comprises an amino acid sequence having at least 85%, at least 90%, at least 91% at least 92%, at least 93%, at least 94%, at least 95%, at least 96%, at least 97%, at least 98%, at least 99%, or 100% sequence identity to SEQ ID NO: 36. In some aspects, the CD8α transmembrane domain comprises an amino acid sequence having at least about 85%, at least about 90%, at least about 91%, at least about 92%, at least about 93%, at least about 94%, at least about 95%, at least about 96%, at least about 97%, at least about 98%, at least about 99%, or 100% sequence identity to SEQ ID NO: 36. In some aspects, the CD8α transmembrane domain comprises an amino acid sequence having about 85%, about 90%, about 91%, about 92%, about 93%, about 94%, about 95%, about 96%, about 97%, about 98%, about 99%, or about 100% sequence identity to SEQ ID NO: 36.

In some aspects, the CD8α transmembrane domain comprises an amino acid sequence at least 85% identical to the amino acid sequence of SEQ ID NO: 36. In some aspects, the CD8α transmembrane domain comprises an amino acid sequence at least 90% identical to the amino acid sequence of SEQ ID NO: 36. In some aspects, the CD8α transmembrane domain comprises an amino acid sequence at least 95% identical to the amino acid sequence of SEQ ID NO: 36. In some aspects, the CD8α transmembrane domain comprises an amino acid sequence at least 98% identical to the amino acid sequence of SEQ ID NO: 36. In some aspects, the CD8α transmembrane domain comprises an amino acid sequence at least 99% identical to the amino acid sequence of SEQ ID NO: 36. In some aspects, the CD8α transmembrane domain comprises an amino acid sequence corresponding to the amino acid sequence of SEQ ID NO: 36. In some aspects, the CD8α transmembrane domain comprises an amino acid sequence about 85% identical to the amino acid sequence SEQ ID NO: 36. In some aspects, the CD8α transmembrane domain comprises an amino acid sequence about 90% identical to the amino acid sequence SEQ ID NO: 36. In some aspects, the CD8α transmembrane domain comprises an amino acid sequence about 95% identical to the amino acid sequence SEQ ID NO: 36. In some aspects, the CD8α transmembrane domain comprises an amino acid sequence about 98% identical to the amino acid sequence SEQ ID NO: 36. In some aspects, the CD8α transmembrane domain comprises an amino acid sequence about 99% identical to the amino acid sequence SEQ ID NO: 36.

In some aspects, the signaling domain comprises a CD3zeta signaling domain. In some aspects, the CD3zeta signaling domain comprises an amino acid sequence having at least 85%, at least 90%, at least 91% at least 92%, at least 93%, at least 94%, at least 95%, at least 96%, at least 97%, at least 98%, at least 99%, or 100% sequence identity to SEQ ID NO: 38. In some aspects, the CD3zeta signaling domain comprises an amino acid sequence having at least about 85%, at least about 90%, at least about 91%, at least about 92%, at least about 93%, at least about 94%, at least about 95%, at least about 96%, at least about 97%, at least about 98%, at least about 99%, or 100% sequence identity to SEQ ID NO: 38. In some aspects, the CD3zeta signaling domain comprises an amino acid sequence having about 85%, about 90%, about 91%, about 92%, about 93%, about 94%, about 95%, about 96%, about 97%, about 98%, about 99%, or about 100% sequence identity to SEQ ID NO: 38.

In some aspects, the CD3zeta signaling domain comprises an amino acid sequence at least 85% identical to the amino acid sequence of SEQ ID NO: 38. In some aspects, the CD3zeta signaling domain comprises an amino acid sequence at least 90% identical to the amino acid sequence of SEQ ID NO: 38. In some aspects, the CD3zeta signaling domain comprises an amino acid sequence at least 95% identical to the amino acid sequence of SEQ ID NO: 38. In some aspects, the CD3zeta signaling domain comprises an amino acid sequence at least 98% identical to the amino acid sequence of SEQ ID NO: 38. In some aspects, the CD3 zeta signaling domain comprises an amino acid sequence at least 99% identical to the amino acid sequence of SEQ ID NO: 38. In some aspects, the CD3zeta signaling domain comprises an amino acid sequence corresponding to the amino acid sequence of SEQ ID NO: 38. In some aspects, the CD3zeta signaling domain comprises an amino acid sequence about 85% identical to the amino acid sequence SEQ ID NO: 38. In some aspects, the CD3zeta signaling domain comprises an amino acid sequence about 90% identical to the amino acid sequence SEQ ID NO: 38. In some aspects, the CD3zeta signaling domain comprises an amino acid sequence about 95% identical to the amino acid sequence SEQ ID NO: 38. In some aspects, the CD3zeta signaling domain comprises an amino acid sequence about 98% identical to the amino acid sequence SEQ ID NO: 38. In some aspects, the CD3zeta signaling domain comprises an amino acid sequence about 99% identical to the amino acid sequence SEQ ID NO: 38.

In some aspects, the chimeric antigen receptor comprises any of the amino acid sequences shown in Table 4 or combinations thereof.

TABLE 4
Exemplary Chimeric Antigen Receptor Amino Acid Sequences

| SEQ ID NO | Name | Amino Acid Sequence |
|---|---|---|
| 1 | Chimeric VH AA | EVQLQQSGAELVKPGASVKLSCTASGFNIKDTYMHWVKQRPEQ |
| | | GLEWIGRIDPANGNTQYDPKFQGKATIPADTSSNTAYLQLSSLTS |
| | | EDTAVYYCVYGRGSAWFAFWGQGTLVTVSA |
| 2 | Chimeric VL AA | DIQMTQTTSSLSASLGDRVTISCRASQDISNYLNWYQQKPDGTV |
| | | KLLIYYTSRLHSGVPSRFSGSGSGTDYSLTISNLEQEDIATYFCQQ |
| | | GNTLPWTFGGGTKLEIK |
| 6 | Chimeric Antigen | MALPVTALLLPLALLLHAARPDIQMTQTTSSLSASLGDRVTISCR |
| | Receptor AA #1 (4-1BB) | ASQDISNYLNWYQQKPDGTVKLLIYYTSRLHSGVPSRFSGSGSG |
| | | TDYSLTISNLEQEDIATYFCQQGNTLPWTFGGGTKLEIKGGGGS |
| | | GGGGSGGGGSEVQLQQSGAELVKPGASVKLSCTASGFNIKDTY |
| | | MHWVKQRPEQGLEWIGRIDPANGNTQYDPKFQGKATIPADTSS |
| | | NTAYLQLSSLTSEDTAVYYCVYGRGSAWFAFWGQGTLVTVSA |
| | | TTTPAPRPPTPAPTIASQPLSLRPEACRPAAGGAVHTRGLDFACD |
| | | IYIWAPLAGTCGVLLLSLVITLYCKRGRKKLLYIFKQPFMRPVQT |
| | | TQEEDGCSCRFPEEEEGGCELRVKFSRSADAPAYQQGQNQLYN |
| | | ELNLGRREEYDVLDKRRGRDPEMGGKPRRKNPQEGLYNELQK |
| | | DKMAEAYSEIGMKGERRRGKGHDGLYQGLSTATKDTYDALHM |
| | | QALPPR |
| 32 | CD8 alpha signal peptide | MALPVTALLLPLALLLHAARP |
| | AA | |
| 33 | scFv AA | DIQMTQTTSSLSASLGDRVTISCRASQDISNYLNWYQQKPDGTV |
| | | KLLIYYTSRLHSGVPSRFSGSGSGTDYSLTISNLEQEDIATYFCQQ |
| | | GNTLPWTFGGGTKLEIKGGGGSGGGGSGGGGSEVQLQQSGAEL |
| | | VKPGASVKLSCTASGFNIKDTYMHWVKQRPEQGLEWIGRIDPA |
| | | NGNTQYDPKFQGKATIPADTSSNTAYLQLSSLTSEDTAVYYCVY |
| | | GRGSAWFAFWGQGTLVTVSA |
| 34 | linker AA | GGGGSGGGGSGGGGS |
| 35 | CD8 alpha hinge domain | TTTPAPRPPTPAPTIASQPLSLRPEACRPAAGGAVHTRGLDFACD |
| | AA | |
| 36 | CD8 alpha transmembrane | IYIWAPLAGTCGVLLLSLVITLYC |
| | domain AA | |
| 37 | 4-1BB costimulatory | KRGRKKLLYIFKQPFMRPVQTTQEEDGCSCRFPEEEEGGCEL |
| | domain AA | |
| 38 | CD3 zeta costimulatory | RVKFSRSADAPAYQQGQNQLYNELNLGRREEYDVLDKRRGRD |
| | domain AA | PEMGGKPRRKNPQEGLYNELQKDKMAEAYSEIGMKGERRRGK |
| | | GHDGLYQGLSTATKDTYDALHMQALPPR |
| 52 | CD28 costimulatory | KRSRLLHSDYMNMTPRRPGPTRKHYQPYAPPRDFAAY |
| | domain AA | |
| 53 | OX40 costimulatory | RRDQRLPPDAHKPPGGGSFRTPIQEEQADAHSTLAKI |
| | domain AA | |
| 56 | Chimeric Antigen Receptor | MALPVTALLLPLALLLHAARPDIQMTQTTSSLSASLGDRVTISCR |
| | AA #2 (CD28) | ASQDISNYLNWYQQKPDGTVKLLIYYTSRLHSGVPSRFSGSGSG |
| | | TDYSLTISNLEQEDIATYFCQQGNTLPWTFGGGTKLEIKGGGGS |
| | | GGGGSGGGGSEVQLQQSGAEL VKPGASVKLSCTASGFNIKDTY |
| | | MHWVKQRPEQGLEWIGRIDPANGNTQYDPKFQGKATIPADTSS |
| | | NTAYLQLSSLTSEDTAVYYCVYGRGSAWFAFWGQGTL VTVSAT |
| | | TTPAPRPPTPAPTIASQPLSLRPEACRPAAGGAVHTRGLDFACDI |
| | | YIWAPLAGTCGVLLLSLVITLYCKRSRLLHSDYMNMTPRRPGPT |
| | | RKHYQPYAPPRDFAAYRVKFSRSADAPAYQQGQNQLYNELNL |
| | | GRREEYDVLDKRRGRDPEMGGKPRRKNPQEGLYNELQKDKMA |
| | | EAYSEIGMKGERRRGKGHDGLYQGLSTATKDTYDALHMQALP |
| | | PR |
| 57 | Chimeric Antigen Receptor | MALPVTALLLPLALLLHAARPDIQMTQTTSSLSASLGDRVTISCR |
| | AA #3 (OX40) | ASQDISNYLNWYQQKPDGTVKLLIYYTSRLHSGVPSRFSGSGSG |
| | | TDYSLTISNLEQEDIATYFCQQGNTLPWTFGGGTKLEIKGGGGS |
| | | GGGGSGGGGSEVQLQQSGAELVKPGASVKLSCTASGFNIKDTY |
| | | MHWVKQRPEQGLEWIGRIDPANGNTQYDPKFQGKATIPADTSS |
| | | NTAYLQLSSLTSEDTAVYYCVYGRGSAWFAFWGQGTL VTVSAT |
| | | TTPAPRPPTPAPTIASQPLSLRPEACRPAAGGAVHTRGLDFACDI |
| | | YIWAPLAGTCGVLLLSLVITLYCRRDQRLPPDAHKPPGGGSFRTP |
| | | IQEEQADAHSTLAKIRVKFSRSADAPAYQQGQNQLYNELNLGR |
| | | REEYDVLDKRRGRDPEMGGKPRRKNPQEGLYNELQKDKMAEA |
| | | YSEIGMKGERRRGKGHDGLYQGLSTATKDTYDALHMQALPPR |

In some aspects, the chimeric antigen receptor comprises any of the nucleic acid sequences shown in Table 5 or combinations thereof.

TABLE 5
Exemplary Chimeric Antigen Receptor Nucleic Acid Sequences

| SEQ ID NO | Name | Nucleic Acid Sequence |
|---|---|---|
| 10 | VH DNA (CAR) | GAGGTGCAGCTGCAACAAAGCGGCGCCGAGCTGGTGAAGCC |
| | | CGGCGCCAGCGTGAAACTGAGCTGTACCGCTAGCGGATTTAA |
| | | CATCAAGGACACATATATGCACTGGGTCAAACAGCGGCCCGA |
| | | GCAGGGCCTCGAATGGATCGGCCGGATCGACCCTGCCAACGG |
| | | CAATACTCAATACGACCCCAAGTTCCAGGGCAAGGCCACAAT |
| | | TCCTGCTGATACATCTTCTAATACCGCCTATCTGCAGCTGAGC |
| | | AGCCTGACCTCCGAGGATACCGCCGTGTACTACTGCGTGTAC |
| | | GGCAGAGGCTCTGCCTGGTTCGCCTTTTGGGGCCAGGGAACC |
| | | CTGGTTACGGTGTCCGCC |
| 11 | VL DNA (CAR) | GACATCCAGATGACACAGACCACCAGCAGCCTGTCAGCTTCT |
| | | CTGGGAGATAGAGTGACCATCAGCTGCAGAGCCAGCCAGGA |
| | | CATCAGCAACTACCTGAACTGGTATCAGCAGAAGCCTGATGG |
| | | CACAGTGAAGCTGCTGATCTACTACACCAGCCGGCTGCACAG |
| | | CGGCGTGCCATCTAGATTCAGCGGAAGCGGCAGCGGCACCG |
| | | ACTACAGCCTCACAATCAGCAACCTGGAACAGGAGGACATC |
| | | GCCACCTACTTCTGCCAGCAGGGCAACACACTGCCTTGGACC |
| | | TTCGGCGGCGGCACCAAGCTGGAAATCAAG |
| 18 | Chimeric Antigen Receptor | ATGGCCCTGCCTGTGACCGCCCTGCTGCTGCCACTGGCCCTGC |
| | DNA #1 (4-1BB) | TGCTGCACGCCGCTAGACCTGACATCCAGATGACACAGACCA |
| | | CCAGCAGCCTGTCAGCTTCTCTGGGAGATAGAGTGACCATCA |
| | | GCTGCAGAGCCAGCCAGGACATCAGCAACTACCTGAACTGGT |
| | | ATCAGCAGAAGCCTGATGGCACAGTGAAGCTGCTGATCTACT |
| | | ACACCAGCCGGCTGCACAGCGGCGTGCCATCTAGATTCAGCG |
| | | GAAGCGGCAGCGGCACCGACTACAGCCTCACAATCAGCAAC |
| | | CTGGAACAGGAGGACATCGCCACCTACTTCTGCCAGCAGGGC |
| | | AACACACTGCCTTGGACCTTCGGCGGCGGCACCAAGCTGGAA |
| | | ATCAAGGGCGGAGGCGGCAGCGGCGGCGGAGGCTCTGGCGG |
| | | CGGAGGTTCCGAGGTGCAGCTGCAACAAAGCGGCGCCGAGC |
| | | TGGTGAAGCCCGGCGCCAGCGTGAAACTGAGCTGTACCGCTA |
| | | GCGGATTTAACATCAAGGACACATATATGCACTGGGTCAAAC |
| | | AGCGGCCCGAGCAGGGCCTCGAATGGATCGGCCGGATCGAC |
| | | CCTGCCAACGGCAATACTCAATACGACCCCAAGTTCCAGGGC |
| | | AAGGCCACAATTCCTGCTGATACATCTTCTAATACCGCCTATC |
| | | TGCAGCTGAGCAGCCTGACCTCCGAGGATACCGCCGTGTACT |
| | | ACTGCGTGTACGGCAGAGGCTCTGCCTGGTTCGCCTTTTGGG |
| | | GCCAGGGAACCCTGGTTACGGTGTCCGCCACAACCACCCCTG |
| | | CCCCTAGACCTCCTACCCCTGCTCCAACCATCGCCAGCCAGC |
| | | CCCTGTCTCTGCGGCCTGAAGCTTGCAGACCCGCTGCTGGCG |
| | | GCGCCGTGCACACGAGGGGACTGGACTTCGCCTGCGACATCT |
| | | ACATTTGGGCCCCTCTGGCCGGCACCTGCGGCGTGCTGCTGC |
| | | TGAGCCTGGTCATCACACTGTACTGCAAGAGAGGCAGAAAA |
| | | AAGCTGCTGTACATCTTCAAGCAGCCTTTTATGCGGCCCGTGC |
| | | AGACCACACAGGAGGAAGATGGCTGCTCTTGTAGATTCCCCG |
| | | AGGAAGAGGAAGGCGGATGTGAACTGAGAGTGAAGTTCAGC |
| | | AGAAGCGCCGATGCCCCTGCCTACCAGCAGGGACAGAACCA |
| | | GCTGTACAACGAGCTGAACCTGGGCAGACGGGAAGAGTACG |
| | | ACGTGCTGGATAAGCGGCGGGGTCGGGACCCCGAGATGGGA |
| | | GGCAAGCCTAGAAGGAAGAACCCCCAAGAGGGCCTTTATAA |
| | | TGAGCTGCAGAAAGATAAGATGGCCGAAGCCTACAGCGAGA |
| | | TCGGCATGAAGGGCGAGAGACGCAGAGGCAAAGGCCACGAC |
| | | GGCCTGTACCAAGGCCTCTCCACCGCCACAAAGGACACCTAC |
| | | GACGCCCTGCACATGCAGGCCCTGCCTCCAAGATGA |
| 39 | CD8 alpha signal peptide | ATGGCCCTGCCTGTGACCGCCCTGCTGCTGCCACTGGCCCTGC |
| | DNA | TGCTGCACGCCGCTAGACCT |
| 40 | scFv DNA | GACATCCAGATGACACAGACCACCAGCAGCCTGTCAGCTTCT |
| | | CTGGGAGATAGAGTGACCATCAGCTGCAGAGCCAGCCAGGA |
| | | CATCAGCAACTACCTGAACTGGTATCAGCAGAAGCCTGATGG |
| | | CACAGTGAAGCTGCTGATCTACTACACCAGCCGGCTGCACAG |
| | | CGGCGTGCCATCTAGATTCAGCGGAAGCGGCAGCGGCACCG |
| | | ACTACAGCCTCACAATCAGCAACCTGGAACAGGAGGACATC |
| | | GCCACCTACTTCTGCCAGCAGGGCAACACACTGCCTTGGACC |
| | | TTCGGCGGCGGCACCAAGCTGGAAATCAAGGGCGGAGGCGG |
| | | CAGCGGCGGCGGAGGCTCTGGCGGCGGAGGTTCCGAGGTGC |
| | | AGCTGCAACAAAGCGGCGCCGAGCTGGTGAAGCCCGGCGCC |
| | | AGCGTGAAACTGAGCTGTACCGCTAGCGGATTTAACATCAAG |
| | | GACACATATATGCACTGGGTCAAACAGCGGCCCGAGCAGGG |
| | | CCTCGAATGGATCGGCCGGATCGACCCTGCCAACGGCAATAC |
| | | TCAATACGACCCCAAGTTCCAGGGCAAGGCCACAATTCCTGC |
| | | TGATACATCTTCTAATACCGCCTATCTGCAGCTGAGCAGCCTG |
| | | ACCTCCGAGGATACCGCCGTGTACTACTGCGTGTACGGCAGA |
| | | GGCTCTGCCTGGTTCGCCTTTTGGGGCCAGGGAACCCTGGTT |
| | | ACGGTGTCCGCC |
| 41 | linker DNA | GGCGGAGGCGGCAGCGGCGGCGGAGGCTCTGGCGGCGGAGG |
| | | TTCC |
| 42 | CD8 alpha hinge domain | ACAACCACCCCTGCCCCTAGACCTCCTACCCCTGCTCCAACC |
| | DNA | ATCGCCAGCCAGCCCCTGTCTCTGCGGCCTGAAGCTTGCAGA |
| | | CCCGCTGCTGGCGGCGCCGTGCACACGAGGGGACTGGACTTC |
| | | GCCTGCGAC |
| 43 | CD8 alpha transmembrane | ATCTACATTTGGGCCCCTCTGGCCGGCACCTGCGGCGTGCTG |
| | domain DNA | CTGCTGAGCCTGGTCATCACACTGTACTGC |
| 44 | 4-1BB costimulatory | AAGAGAGGCAGAAAAAAGCTGCTGTACATCTTCAAGCAGCC |
| | domain DNA | TTTTATGCGGCCCGTGCAGACCACACAGGAGGAAGATGGCTG |
| | | CTCTTGTAGATTCCCCGAGGAAGAGGAAGGCGGATGTGAACT |
| | | G |
| 45 | CD3 zeta costimulatory | AGAGTGAAGTTCAGCAGAAGCGCCGATGCCCCTGCCTACCAG |
| | domain DNA | CAGGGACAGAACCAGCTGTACAACGAGCTGAACCTGGGCAG |
| | | ACGGGAAGAGTACGACGTGCTGGATAAGCGGCGGGGTCGGG |
| | | ACCCCGAGATGGGAGGCAAGCCTAGAAGGAAGAACCCCCAA |
| | | GAGGGCCTTTATAATGAGCTGCAGAAAGATAAGATGGCCGA |
| | | AGCCTACAGCGAGATCGGCATGAAGGGCGAGAGACGCAGAG |
| | | GCAAAGGCCACGACGGCCTGTACCAAGGCCTCTCCACCGCCA |
| | | CAAAGGACACCTACGACGCCCTGCACATGCAGGCCCTGCCTC |
| | | CAAGATGA |
| 54 | CD28 costimulatory | AAACGCAGCCGCCTGCTGCATAGCGATTATATGAACATGACC |
| | domain DNA | CCGCGCCGCCCGGGCCCGACCCGCAAACATTATCAGCCGTAT |
| | | GCGCCGCCGCGCGATTTTGCGGCGTAT |
| 55 | OX40 costimulatory | CGGAGGGACCAGAGGCTGCCCCCCGATGCCCACAAGCCCCCT |
| | domain DNA | GGGGGAGGCAGTTTCCGGACCCCCATCCAAGAGGAGCAGGC |
| | | CGACGCCCACTCCACCCTGGCCAAGATC |
| 58 | Chimeric Antigen Receptor | ATGGCCCTGCCTGTGACCGCCCTGCTGCTGCCACTGGCCCTGC |
| | DNA #2 (CD28) | TGCTGCACGCCGCTAGACCTGACATCCAGATGACACAGACCA |
| | | CCAGCAGCCTGTCAGCTTCTCTGGGAGATAGAGTGACCATCA |
| | | GCTGCAGAGCCAGCCAGGACATCAGCAACTACCTGAACTGGT |
| | | ATCAGCAGAAGCCTGATGGCACAGTGAAGCTGCTGATCTACT |
| | | ACACCAGCCGGCTGCACAGCGGCGTGCCATCTAGATTCAGCG |
| | | GAAGCGGCAGCGGCACCGACTACAGCCTCACAATCAGCAAC |
| | | CTGGAACAGGAGGACATCGCCACCTACTTCTGCCAGCAGGGC |
| | | AACACACTGCCTTGGACCTTCGGCGGCGGCACCAAGCTGGAA |
| | | ATCAAGGGCGGAGGCGGCAGCGGCGGCGGAGGCTCTGGCGG |
| | | CGGAGGTTCCGAGGTGCAGCTGCAACAAAGCGGCGCCGAGC |
| | | TGGTGAAGCCCGGCGCCAGCGTGAAACTGAGCTGTACCGCTA |
| | | GCGGATTTAACATCAAGGACACATATATGCACTGGGTCAAAC |
| | | AGCGGCCCGAGCAGGGCCTCGAATGGATCGGCCGGATCGAC |
| | | CCTGCCAACGGCAATACTCAATACGACCCCAAGTTCCAGGGC |
| | | AAGGCCACAATTCCTGCTGATACATCTTCTAATACCGCCTATC |
| | | TGCAGCTGAGCAGCCTGACCTCCGAGGATACCGCCGTGTACT |
| | | ACTGCGTGTACGGCAGAGGCTCTGCCTGGTTCGCCTTTTGGG |
| | | GCCAGGGAACCCTGGTTACGGTGTCCGCCACAACCACCCCTG |
| | | CCCCTAGACCTCCTACCCCTGCTCCAACCATCGCCAGCCAGC |
| | | CCCTGTCTCTGCGGCCTGAAGCTTGCAGACCCGCTGCTGGCG |
| | | GCGCCGTGCACACGAGGGGACTGGACTTCGCCTGCGACATCT |
| | | ACATTTGGGCCCCTCTGGCCGGCACCTGCGGCGTGCTGCTGC |
| | | TGAGCCTGGTCATCACACTGTACTGCAAACGCAGCCGCCTGC |
| | | TGCATAGCGATTATATGAACATGACCCCGCGCCGCCCGGGCC |
| | | CGACCCGCAAACATTATCAGCCGTATGCGCCGCCGCGCGATT |
| | | TTGCGGCGTATAGAGTGAAGTTCAGCAGAAGCGCCGATGCCC |
| | | CTGCCTACCAGCAGGGACAGAACCAGCTGTACAACGAGCTG |
| | | AACCTGGGCAGACGGGAAGAGTACGACGTGCTGGATAAGCG |
| | | GCGGGGTCGGGACCCCGAGATGGGAGGCAAGCCTAGAAGGA |
| | | AGAACCCCCAAGAGGGCCTTTATAATGAGCTGCAGAAAGAT |
| | | AAGATGGCCGAAGCCTACAGCGAGATCGGCATGAAGGGCGA |
| | | GAGACGCAGAGGCAAAGGCCACGACGGCCTGTACCAAGGCC |
| | | TCTCCACCGCCACAAAGGACACCTACGACGCCCTGCACATGC |
| | | AGGCCCTGCCTCCAAGATGA |
| 59 | Chimeric Antigen Receptor | ATGGCCCTGCCTGTGACCGCCCTGCTGCTGCCACTGGCCCTGC |
| | DNA #3 (OX40) | TGCTGCACGCCGCTAGACCTGACATCCAGATGACACAGACCA |
| | | CCAGCAGCCTGTCAGCTTCTCTGGGAGATAGAGTGACCATCA |
| | | GCTGCAGAGCCAGCCAGGACATCAGCAACTACCTGAACTGGT |
| | | ATCAGCAGAAGCCTGATGGCACAGTGAAGCTGCTGATCTACT |
| | | ACACCAGCCGGCTGCACAGCGGCGTGCCATCTAGATTCAGCG |
| | | GAAGCGGCAGCGGCACCGACTACAGCCTCACAATCAGCAAC |
| | | CTGGAACAGGAGGACATCGCCACCTACTTCTGCCAGCAGGGC |
| | | AACACACTGCCTTGGACCTTCGGCGGCGGCACCAAGCTGGAA |
| | | ATCAAGGGCGGAGGCGGCAGCGGCGGCGGAGGCTCTGGCGG |
| | | CGGAGGTTCCGAGGTGCAGCTGCAACAAAGCGGCGCCGAGC |
| | | TGGTGAAGCCCGGCGCCAGCGTGAAACTGAGCTGTACCGCTA |
| | | GCGGATTTAACATCAAGGACACATATATGCACTGGGTCAAAC |
| | | AGCGGCCCGAGCAGGGCCTCGAATGGATCGGCCGGATCGAC |
| | | CCTGCCAACGGCAATACTCAATACGACCCCAAGTTCCAGGGC |
| | | AAGGCCACAATTCCTGCTGATACATCTTCTAATACCGCCTATC |
| | | TGCAGCTGAGCAGCCTGACCTCCGAGGATACCGCCGTGTACT |
| | | ACTGCGTGTACGGCAGAGGCTCTGCCTGGTTCGCCTTTTGGG |
| | | GCCAGGGAACCCTGGTTACGGTGTCCGCCACAACCACCCCTG |
| | | CCCCTAGACCTCCTACCCCTGCTCCAACCATCGCCAGCCAGC |
| | | CCCTGTCTCTGCGGCCTGAAGCTTGCAGACCCGCTGCTGGCG |
| | | GCGCCGTGCACACGAGGGGACTGGACTTCGCCTGCGACATCT |
| | | ACATTTGGGCCCCTCTGGCCGGCACCTGCGGCGTGCTGCTGC |
| | | TGAGCCTGGTCATCACACTGTACTGCCGGAGGGACCAGAGGC |
| | | TGCCCCCCGATGCCCACAAGCCCCCTGGGGGAGGCAGTTTCC |
| | | GGACCCCCATCCAAGAGGAGCAGGCCGACGCCCACTCCACCC |
| | | TGGCCAAGATCAGAGTGAAGTTCAGCAGAAGCGCCGATGCC |
| | | CCTGCCTACCAGCAGGGACAGAACCAGCTGTACAACGAGCTG |
| | | AACCTGGGCAGACGGGAAGAGTACGACGTGCTGGATAAGCG |
| | | GCGGGGTCGGGACCCCGAGATGGGAGGCAAGCCTAGAAGGA |
| | | AGAACCCCCAAGAGGGCCTTTATAATGAGCTGCAGAAAGAT |
| | | AAGATGGCCGAAGCCTACAGCGAGATCGGCATGAAGGGCGA |
| | | GAGACGCAGAGGCAAAGGCCACGACGGCCTGTACCAAGGCC |
| | | TCTCCACCGCCACAAAGGACACCTACGACGCCCTGCACATGC |
| | | AGGCCCTGCCTCCAAGATGA |

In some aspects, the binding domain or the extracellular domain of the CAR provides the CAR with the ability to bind to the target antigen of interest. A binding domain (e.g., a ligand-binding domain or antigen-binding domain) can be any protein, polypeptide, oligopeptide, or peptide that possesses the ability to specifically recognize and bind to a biological molecule (e.g., a cell surface receptor or tumor protein, or a component thereof). A binding domain can include any naturally occurring, synthetic, semi-synthetic, or recombinantly produced binding partner for a biological molecule of interest. For example, and as further disclosed herein, a binding domain can be antibody light chain and heavy chain variable regions, or the light and heavy chain variable regions can be joined together in a single chain and in either orientation (e.g., VL-VH or VH-VL). A variety of assays are known for identifying binding domains of the present disclosure that specifically bind with a particular target, including Western blot, ELISA, flow cytometry, or surface plasmon resonance analysis (e.g., using BIACORE analysis). The target can be an antigen of clinical interest against which it would be desirable to trigger an effector immune response that results in tumor killing. In some aspects, the target antigen of the binding domain of the chimeric antigen receptor is a CD94 protein.

Illustrative ligand-binding domains include antigen binding proteins, such as antigen binding fragments of an antibody, such as scFv, extracellular domains of receptors, ligands for cell surface molecules/receptors, or receptor binding domains thereof, and tumor binding proteins. In some aspects, the antigen binding domains included in a CAR of the disclosure can be a variable region (Fv), a CDR, a Fab, an scFv, a VH, a VL, a domain antibody variant (dAb), a camelid antibody (VHH) and other antigen-specific binding domain derived from other protein scaffolds.

In some aspects, the binding domain of the CAR is an anti-CD94 single chain antibody (scFv), and can be a murine, chimeric, human or humanized scFv. Single chain antibodies can be cloned from the V region genes of a hybridoma specific for a desired target. A technique which can be used for cloning the variable region heavy chain (VH) and variable region light chain (VL) has been disclosed, for example, in Orlandi et al., PNAS, 1989; 86:3833-3837. Thus, in some aspects, a binding domain comprises an antibody-derived binding domain but can be a non-antibody derived binding domain. An antibody-derived binding domain can be a fragment of an antibody or a genetically engineered product of one or more fragments of the antibody, which fragment is involved in binding with the antigen.

In some aspects, the CARs of the present disclosure can comprise a linker between one or more domains, e.g., added for appropriate spacing and conformation of the molecule. For example, in some aspects, there may be a linker between the binding domain VH or VL. In some aspects, the linker can be between 1-10 amino acids long. In some aspects, the linker between any of the domains of the chimeric antigen receptor can be between 1-20 or 20 amino acids long. In this regard, the linker can be 1, 2, 3, 4, 5, 6, 7, 8, 9, 10, 11, 12, 13, 14, 15, 16, 17, 18, 19 or 20 amino acids long. In some aspects, the linker can be 21, 22, 23, 24, 25, 26, 27, 28, 29 or 30 amino acids long. Ranges including the numbers described herein are also included herein, e.g., a linker 10-30 amino acids long.

In some aspects, linkers suitable for use in the CAR disclosed herein are flexible linkers. Suitable linkers can be readily selected and can be of any of a suitable of different lengths, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and can be 1, 2, 3, 4, 5, 6, or 7 amino acids.

Exemplary flexible linkers can include glycine polymers (G) n, glycine-serine polymers, where n is an integer of at least one, glycine-alanine polymers, alanine-serine polymers, and other flexible linkers known in the art. Glycine and glycine-serine polymers are relatively unstructured, and therefore can be able to serve as a neutral tether between domains of fusion proteins such as the CARs disclosed herein. Glycine accesses significantly more phi-psi space than even alanine, and is much less restricted than residues with longer side chains (see Scheraga, Rev. Computational Chem. 11173-142 (1992)). The ordinarily skilled artisan will recognize that design of a CAR can include linkers that are all or partially flexible, such that the linker can include a flexible linker as well as one or more portions that confer less flexible structure to provide for a desired CAR structure. Specific linkers include (G4S)n linkers, wherein n=1-3. In some aspects, the linker comprises an amino acid sequence having at least 85%, at least 90%, at least 91% at least 92%, at least 93%, at least 94%, at least 95%, at least 96%, at least 97%, at least 98%, at least 99%, or 100% sequence identity to SEQ ID NO: 34. In some aspects, the linker comprises an amino acid sequence having at least about 85%, at least about 90%, at least about 91%, at least about 92%, at least about 93%, at least about 94%, at least about 95%, at least about 96%, at least about 97%, at least about 98%, at least about 99%, or 100% sequence identity to SEQ ID NO: 34. In some aspects, the linker comprises an amino acid sequence having about 85%, about 90%, about 91%, about 92%, about 93%, about 94%, about 95%, about 96%, about 97%, about 98%, about 99%, or about 100% sequence identity to SEQ ID NO: 34.

In some aspects, the linker comprises an amino acid sequence at least 85% identical to the amino acid sequence of SEQ ID NO: 34. In some aspects, the linker comprises an amino acid sequence at least 90% identical to the amino acid sequence of SEQ ID NO: 34. In some aspects, the linker comprises an amino acid sequence at least 95% identical to the amino acid sequence of SEQ ID NO: 34. In some aspects, the linker comprises an amino acid sequence at least 98% identical to the amino acid sequence of SEQ ID NO: 34. In some aspects, the linker comprises an amino acid sequence at least 99% identical to the amino acid sequence of SEQ ID NO: 34. In some aspects, the linker comprises an amino acid sequence corresponding to the amino acid sequence of SEQ ID NO: 34. In some aspects, the linker comprises an amino acid sequence about 85% identical to the amino acid sequence SEQ ID NO: 34. In some aspects, the linker comprises an amino acid sequence about 90% identical to the amino acid sequence SEQ ID NO: 34. In some aspects, the linker comprises an amino acid sequence about 95% identical to the amino acid sequence SEQ ID NO: 34. In some aspects, the linker comprises an amino acid sequence about 98% identical to the amino acid sequence SEQ ID NO: 34. In some aspects, the linker comprises an amino acid sequence about 99% identical to the amino acid sequence SEQ ID NO: 34.

In some aspects, the transmembrane region or domain is the portion of the CAR that anchors the extracellular binding portion to the plasma membrane of the immune effector cell, and facilitates binding of the binding domain to the target antigen. The transmembrane domain can be a CD3zeta transmembrane domain, however other transmembrane domains that can be employed include those obtained from CD8α, CD4, CD28, CD45, CD9, CD16, CD22, CD33, CD64, CD80, CD86, CD134, CD137, and CD154. In some aspects, the transmembrane domain is the transmembrane domain of CD8a. In some aspects, the transmembrane domain is synthetic in which case it would comprise predominantly hydrophobic residues such as leucine and valine.

Signals generated through the T cell receptor alone are insufficient for full activation of a T cell. A costimulatory signal or signaling domain should also be present to activate the T cell.

Examples of signal transducing domains for use in a CAR can be the cytoplasmic sequences of the T cell receptor and co-receptors that act in concert to initiate signal transduction following antigen receptor engagement, as well as any derivate or variant of these sequences and any synthetic sequence that has the same functional capability. In some cases, signaling domains comprise two distinct classes of cytoplasmic signaling sequences, those that initiate antigen-dependent primary activation, and those that act in an antigen-independent manner to provide a secondary or co-stimulatory signal. Primary cytoplasmic signaling sequences can comprise signaling motifs that are known as immunoreceptor tyrosine-based activation motifs (ITAMs). ITAMs can be found in the intracytoplasmic tail of a variety of receptors that serve as binding sites for syk/zap70 class tyrosine kinases.

Exemplary ITAMs can include those derived from TCRzeta, FcRgamma, FcRbeta, FcRepsilon, CD3gamma, CD3delta, CD3epsilon, CD3zeta, CD5, CD22, CD79a, CD79b and CD66d. In some aspects, the signal transducing domain of the CAR can comprise the CD3zeta signaling domain.

In some aspects, the CAR disclosed herein can comprise a costimulatory domain, e.g., derived from a costimulatory molecule. Costimulatory molecules can include, but are not limited to, an MHC class I molecule, BTLA and Toll ligand receptor. Examples of costimulatory molecules include, e.g., CD27, CD28, CD8, 4-1BB (CD137), OX40, CD30, CD40, PD-1, ICOS, lymphocyte function-associated antigen-1 (LFA-1), CD2, CD7, LIGHT, NKG2C, B7-H3 and a ligand that specifically binds with CD83 and the like. A costimulatory molecule can be a cell surface molecule other than an antigen receptor or their ligands that can contribute to an efficient immune response. Accordingly, while the present disclosure provides exemplary costimulatory domains derived from 4-1BB, other costimulatory domains are contemplated for use with the CARs disclosed herein. The inclusion of one or more co-stimulatory signaling domains can enhance the efficacy and expansion of T cells expressing CAR receptors disclosed herein. The intracellular signaling and costimulatory signaling domains can be linked in any order in tandem to the carboxyl terminus of the transmembrane domain.

In some aspects, the CAR disclosed herein can comprises a signal peptide. In some aspects, the signal peptide is a CD8α signal peptide. In some aspects, the CD8α signal peptide comprises an amino acid sequence having at least 85%, at least 90%, at least 91% at least 92%, at least 93%, at least 94%, at least 95%, at least 96%, at least 97%, at least 98%, at least 99%, or 100% sequence identity to SEQ ID NO: 32. In some aspects, the CD8α signal peptide comprises an amino acid sequence having at least about 85%, at least about 90%, at least about 91%, at least about 92%, at least about 93%, at least about 94%, at least about 95%, at least about 96%, at least about 97%, at least about 98%, at least about 99%, or 100% sequence identity to SEQ ID NO: 32. In some aspects, the CD8α signal peptide comprises an amino acid sequence having about 85%, about 90%, about 91%, about 92%, about 93%, about 94%, about 95%, about 96%, about 97%, about 98%, about 99%, or about 100% sequence identity to SEQ ID NO: 32.

In some aspects, the CD8α signal peptide comprises an amino acid sequence at least 85% identical to the amino acid sequence of SEQ ID NO: 32. In some aspects, the CD8α signal peptide comprises an amino acid sequence at least 90% identical to the amino acid sequence of SEQ ID NO: 32. In some aspects, the CD8α signal peptide comprises an amino acid sequence at least 95% identical to the amino acid sequence of SEQ ID NO: 32. In some aspects, the CD8α signal peptide comprises an amino acid sequence at least 98% identical to the amino acid sequence of SEQ ID NO: 32. In some aspects, the CD8α signal peptide comprises an amino acid sequence at least 99% identical to the amino acid sequence of SEQ ID NO: 32. In some aspects, the CD8α signal peptide comprises an amino acid sequence corresponding to the amino acid sequence of SEQ ID NO: 32. In some aspects, the CD8α signal peptide comprises an amino acid sequence about 85% identical to the amino acid sequence SEQ ID NO: 32. In some aspects, the CD8α signal peptide comprises an amino acid sequence about 90% identical to the amino acid sequence SEQ ID NO: 32. In some aspects, the CD8α signal peptide comprises an amino acid sequence about 95% identical to the amino acid sequence SEQ ID NO: 32. In some aspects, the CD8α signal peptide comprises an amino acid sequence about 98% identical to the amino acid sequence SEQ ID NO: 32. In some aspects, the CD8α signal peptide comprises an amino acid sequence about 99% identical to the amino acid sequence SEQ ID NO: 32.

Although scFv-based CARs engineered to contain a signaling domain from CD3 or FcRgamma have been shown to deliver a potent signal for T cell activation and effector function, they are not sufficient to elicit signals that promote T cell survival and expansion in the absence of a concomitant costimulatory signal. Other CARs containing a binding domain, a hinge, a transmembrane and the signaling domain derived from CD3zeta or FcRgamma together with one or more costimulatory signaling domains (e.g., intracellular costimulatory domains derived from CD28, CD137, CD134 and CD278) can more effectively direct antitumor activity as well as increased cytokine secretion, lytic activity, survival and proliferation in CAR expressing T cells in vitro, and in animal models and cancer patients (Milone et al., Molecular Therapy, 2009; 17:1453-1464; Zhong et al., Molecular Therapy, 2010; 18:413-420; Carpenito et al., PNAS, 2009; 106:3360-3365).

In some aspects, the CD94-binding CARs of the disclosure comprise (a) an anti-CD94 scFv as a binding domain (e.g., an scFv having binding regions (e.g., CDRs or variable domains) from an anti-CD94 antibody or antigen-binding fragment thereof disclosed herein); (b) a hinge region (e.g., derived from human CD8α); (c) a transmembrane domain (e.g., a human CD8α transmembrane domain); and (d) a signaling domain (e.g., a human T cell receptor CD3zeta chain signaling domain), and optionally one or more costimulatory signaling domains, e.g., a 4-1BB co-stimulatory domain, a CD28 costimulatory domain, or an OX40 costimulatory domain. In some aspects, the different protein domains are arranged from amino to carboxyl terminus in the following order: binding domain, hinge and transmembrane domain. The intracellular signaling domain and optional co-stimulatory signaling domains are linked to the transmembrane carboxy terminus in any order in tandem to form a single chain chimeric polypeptide.

In some aspects, a nucleic acid construct encoding a CD94-binding CAR is a chimeric nucleic acid molecule comprising different coding sequences, for example, (5′ to 3′) the coding sequences of a human anti-CD94 scFv, a human CD8α-hinge region, a human CD8α transmembrane domain and a CD3zeta signaling domain. In some aspects, a nucleic acid construct encoding a CD94-binding CAR is a chimeric nucleic acid molecule comprising different coding sequences, for example, (5′ to 3′) the coding sequences of a human anti-CD94 scFv, a human CD8α-hinge, a human CD8α transmembrane domain, a 4-1BB co-stimulatory domain, and a CD3zeta signaling domain.

In some aspects, the polynucleotide encoding the CAR disclosed herein is inserted into a vector. In some aspects, the vector as used herein is a vehicle into which a polynucleotide encoding a protein can be covalently inserted so as to bring about the expression of that protein and/or the cloning of the polynucleotide. The isolated polynucleotide can be inserted into a vector using any suitable methods known in the art, for example, without limitation, the vector can be digested using appropriate restriction enzymes and then can be ligated with the isolated polynucleotide having matching restriction ends. In some aspects, expression vectors have the ability to incorporate and express heterologous or modified nucleic acid sequences coding for at least part of a gene product capable of being transcribed in a cell. In most cases, RNA molecules are then translated into a protein. Expression vectors can contain a variety of control sequences, which refer to nucleic acid sequences for the transcription and possibly translation of an operatively linked coding sequence in a particular host organism. In addition to control sequences that govern transcription and translation, vectors and expression vectors can contain nucleic acid sequences that serve other functions as well and are discussed infra. An expression vector can comprise additional elements, for example, the expression vector can have two replication systems, thus allowing it to be maintained in two organisms, for example in human cells for expression and in a prokaryotic host for cloning and amplification.

The expression vector can have 5′ upstream and 3′ downstream regulatory elements such as promoter sequences (e.g., CMV, PGK or EF1alpha promoters), ribosome recognition and binding TATA box, and 3′ UTR AAUAAA transcription termination sequence for the efficient gene transcription and translation in its respective host cell. Other suitable promoters can include the constitutive promoter of simian virus 40 (SV40) early promoter, mouse mammary tumor virus (MMTV), HIV LTR promoter, MoMuLV promoter, avian leukemia virus promoter, EBV immediate early promoter, and rous sarcoma virus promoter. Human gene promoters can also be used, including, but not limited to the actin promoter, the myosin promoter, the hemoglobin promoter, and the creatine kinase promoter. In some aspects, inducible promoters are also contemplated as part of the vectors expressing chimeric antigen receptor. This provides a molecular switch capable of turning on expression of the polynucleotide sequence of interest or turning off expression. Examples of inducible promoters include, but are not limited to a metallothionine promoter, a glucocorticoid promoter, a progesterone promoter, or a tetracycline promoter.

The expression vector can have additional sequence such as 6×-histidine, c-Myc, and FLAG tags that are incorporated into the expressed CARs. Thus, the expression vector can be engineered to contain 5′ and 3′ untranslated regulatory sequences that sometimes can function as enhancer sequences, promoter regions and/or terminator sequences that can facilitate or enhance efficient transcription of the nucleic acid(s) of interest carried on the expression vector. An expression vector can also be engineered for replication and/or expression functionality (e.g., transcription and translation) in a particular cell type, cell location, or tissue type. Expression vectors can include a selectable marker for maintenance of the vector in the host or recipient cell.

In some aspects, the vectors are plasmid, autonomously replicating sequences, and transposable elements. Additional exemplary vectors include, without limitation, plasmids, phagemids, cosmids, artificial chromosomes such as yeast artificial chromosome (YAC), bacterial artificial chromosome (BAC), or P1-derived artificial chromosome (PAC), bacteriophages such as lambda phage or M13 phage, and animal viruses. Examples of categories of animal viruses useful as vectors include, without limitation, retrovirus (including lentivirus), adenovirus, adeno-associated virus, herpesvirus (e.g., herpes simplex virus), poxvirus, baculovirus, papillomavirus, and papovavirus (e.g., SV40). Examples of expression vectors are Lenti-X™ Bicistronic Expression System (Neo) vectors (Clontrch), pClneo vectors (Promega) for expression in mammalian cells; pLenti4/V5-DEST™, pLenti6/V5-DEST™, and pLenti6.2N5-GW/lacZ (Invitrogen) for lentivirus-mediated gene transfer and expression in mammalian cells. The coding sequences of the CARs disclosed herein can be ligated into such expression vectors for the expression of the chimeric protein in mammalian cells.

In some aspects, for expression of a CD94-binding CAR, the vector can be introduced into a host cell to allow expression of the polypeptide within the host cell. The expression vectors can contain a variety of elements for controlling expression, including without limitation, promoter sequences, transcription initiation sequences, enhancer sequences, selectable markers, and signal sequences. These elements can be selected as appropriate by a person of ordinary skill in the art, as disclosed herein. For example, the promoter sequences can be selected to promote the transcription of the polynucleotide in the vector. Suitable promoter sequences include, without limitation, T7 promoter, T3 promoter, SP6 promoter, beta-actin promoter, EF1a promoter, CMV promoter, and SV40 promoter. Enhancer sequences can be selected to enhance the transcription of the polynucleotide. Selectable markers can be selected to allow selection of the host cells inserted with the vector from those not, for example, the selectable markers can be genes that confer antibiotic resistance. Signal sequences can be selected to allow the expressed polypeptide to be transported outside of the host cell.

For cloning of the polynucleotide, the vector can be introduced into a host cell (an isolated host cell) to allow replication of the vector itself and thereby amplify the copies of the polynucleotide contained therein. The cloning vectors can contain sequence components generally include, without limitation, an origin of replication, promoter sequences, transcription initiation sequences, enhancer sequences, and selectable markers. These elements can be selected as appropriate by a person of ordinary skill in the art. For example, the origin of replication can be selected to promote autonomous replication of the vector in the host cell.

In some aspects, the present disclosure provides isolated host cells containing the vectors provided herein. The host cells containing the vector can be useful in expression or cloning of the polynucleotide contained in the vector. Suitable host cells can include, without limitation, prokaryotic cells, fungal cells, yeast cells, or higher eukaryotic cells such as mammalian cells. Suitable prokaryotic cells for this purpose include, without limitation, eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis, Pseudomonas such as P. aeruginosa, and Streptomyces.

The CARs of the present disclosure can be introduced into a host cell using transfection and/or transduction techniques known in the art. As used herein, the terms, “transfection,” and, “transduction,” refer to the processes by which an exogenous nucleic acid sequence is introduced into a host cell. The nucleic acid can be integrated into the host cell DNA or can be maintained extrachromosomally. The nucleic acid can be maintained transiently or can be a stable introduction. Transfection can be accomplished by a variety of means known in the art including but not limited to calcium phosphate-DNA co-precipitation, DEAE-dextran-mediated transfection, polybrene-mediated transfection, electroporation, microinjection, liposome fusion, lipofection, protoplast fusion, retroviral infection, and biolistics. Transduction refers to the delivery of a gene(s) using a viral or retroviral vector by means of viral infection rather than by transfection. In some aspects, retroviral vectors are transduced by packaging the vectors into virions prior to contact with a cell. For example, a nucleic acid encoding a CD94 CAR carried by a retroviral vector can be transduced into a cell through infection and pro virus integration.

In some aspects, the CAR of the present disclosure is introduced and expressed in immune effector cells so as to redirect their specificity to a target antigen of interest.

The present disclosure provides methods for making the immune effector cells that express the CAR as disclosed herein. In some aspects, the immune effector cells are isolated from an individual and genetically modified without further manipulation in vitro. Such cells can then be directly re-administered into the individual. In some aspects, the immune effector cells are first activated and stimulated to proliferate in vitro prior to being genetically modified to express a CAR. In this regard, the immune effector cells can be cultured before or after being genetically modified (i.e., transduced or transfected to express a CAR as disclosed herein).

Prior to in vitro manipulation or genetic modification of the immune effector cells disclosed herein, the source of cells can be obtained from a subject. In particular, the immune effector cells for use with the CARs as disclosed herein comprise T cells. T cells can be obtained from a number of sources, including peripheral blood mononuclear cells, bone marrow, lymph nodes tissue, cord blood, thymus issue, tissue from a site of infection, ascites, pleural effusion, spleen tissue, and tumors. In some aspects, T cells can be obtained from a unit of blood collected from the subject using any number of techniques known to the skilled person, such as FICOLL separation. In some aspects, cells from the circulating blood of an individual are obtained by apheresis. The apheresis product typically contains lymphocytes, including T cells, monocytes, granulocyte, B cells, other nucleated white blood cells, red blood cells, and platelets. In some aspects, the cells collected by apheresis can be washed to remove the plasma fraction and to place the cells in an appropriate buffer or media for subsequent processing. In some aspects, the cells are washed with PBS. In some aspects, the washed solution lacks calcium, and can lack magnesium or can lack many, if not all, divalent cations. As would be appreciated by those of ordinary skill in the art, a washing step can be accomplished by methods known to those in the art, such as by using a semiautomated flowthrough centrifuge. After washing, the cells can be resuspended in a variety of biocompatible buffers or other saline solution with or without buffer. In some aspects, the undesirable components of the apheresis sample can be removed in the cell directly resuspended culture media.

In some aspects, T cells are isolated from peripheral blood mononuclear cells (PBMCs) by lysing the red blood cells and depleting the monocytes, for example, by centrifugation through a PERCOLL™ gradient. A specific subpopulation of T cells, such as CD28+, CD4+, CD8+, CD45RA+, and CD45RO+ T cells, can be further isolated by positive or negative selection techniques. For example, enrichment of a T cell population by negative selection can be accomplished with a combination of antibodies directed to surface markers unique to the negatively selected cells. One method for use herein is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD1b, CD16, HLA-DR, and CD8. Flow cytometry and cell sorting can also be used to isolate cell populations of interest for use in the present disclosure.

PBMCs can be used directly for genetic modification with the CARs using methods as disclosed herein. In some aspects, after isolation of PBMC, T lymphocytes are further isolated and in some aspects, both cytotoxic and helper T lymphocytes can be sorted into naive, memory, and effector T cell subpopulations either before or after genetic modification and/or expansion. CD8+ cells can be obtained by using standard methods. In some aspects, CD8+ cells are further sorted into naive, central memory, and effector cells by identifying cell surface antigens that are associated with each of those types of CD8+ cells. In some aspects, memory T cells are present in both CD62L+ and CD62L-subsets of CD8+ peripheral blood lymphocytes. PBMC are sorted into CD62L-CD8+ and CD62L+CD8+ fractions after staining with anti-CD8 and anti-CD62L antibodies. In some aspects, the expression of phenotypic markers of central memory TCM include CD45RO, CD62L, CCR7, CD28, CD3, and CD127 and are negative for granzyme B. In some aspects, central memory T cells are CD45RO+, CD62L+, CD8+ T cells. In some aspects, effector T cells are negative for CD62L, CCR7, CD28, and CD127, and positive for granzyme B and perforin. In some aspects, naive CD8+ T lymphocytes are characterized by the expression of phenotypic markers of naive T cells including CD62L, CCR7, CD28, CD3, CD 127, and CD45RA.

In some aspects, CD4+ T cells are further sorted into subpopulations. For example, CD4+ T helper cells can be sorted into naive, central memory, and effector cells by identifying cell populations that have cell surface antigens. CD4+ lymphocytes can be obtained by standard methods.

The immune effector cells, such as T cells, can be genetically modified following isolation using known methods, or the immune effector cells can be activated and expanded (or differentiated in the case of progenitors) in vitro prior to being genetically modified. In another embodiment, the immune effector cells, such as T cells, are genetically modified with the chimeric antigen receptors disclosed herein (e.g., transduced with a viral vector comprising a nucleic acid encoding a CAR) and then are activated and expanded in vitro. Methods for activating and expanding T cells are known in the art and are disclosed, for example, in U.S. Pat. Nos. 6,905,874; 6,867,041; 6,797,514; WO2012079000. Generally, such methods include contacting PBMC or isolated T cells with a stimulatory agent and costimulatory agent, such as anti-CD3 and anti-CD28 antibodies, generally attached to a bead or other surface, in a culture medium with appropriate cytokines, such as IL-2. Anti-CD3 and anti-CD28 antibodies attached to the same bead serve as a “surrogate” antigen presenting cell (APC). In some aspects, the T cells can be activated and stimulated to proliferate with feeder cells and appropriate antibodies and cytokines using methods such as those disclosed in U.S. Pat. Nos. 6,040,177; 5,827,642; and WO2012129514.

CAR-expressing immune effector cells prepared as disclosed herein can be utilized in methods and compositions for adoptive immunotherapy in accordance with known techniques, or variations thereof that will be apparent to those skilled in the art based on the instant disclosure. See, e.g., US Patent Application Publication No. 2003/0170238 to Gruenberg et al; see also U.S. Pat. No. 4,690,915 to Rosenberg.

In some aspects, the cells are formulated by first harvesting them from their culture medium, and then washing and concentrating the cells in a medium and container system suitable for administration (a “pharmaceutically acceptable” carrier) in a treatment-effective amount. Suitable infusion medium can be any isotonic medium formulation, typically normal saline, Normosol® (Abbott) or Plasma-Lyte A (Baxter), but also 5% dextrose in water or Ringer's lactate can be utilized. The infusion medium can be supplemented with human serum albumin.

The cells can be autologous or heterologous to the patient undergoing therapy. If desired, the treatment can also include administration of mitogens (e.g., PHA) or lymphokines, cytokines, and/or chemokines (e.g., IFN-γ, IL-2, IL-12, TNF-α, IL-18, and TNF-β, GM-CSF, IL-4, IL-13, Flt3-L, RANTES, MIP1α, etc.) as disclosed herein to enhance induction of the immune response.

The CAR expressing immune effector cell populations of the present disclosure can be administered either alone, or as a pharmaceutical composition in combination with diluents and/or with other components such as IL-2 or other cytokines or cell populations. Briefly, pharmaceutical compositions of the present disclosure can comprise a CAR-expressing immune effector cell population, such as T cells, as disclosed herein, in combination with one or more pharmaceutically or physiologically acceptable carriers, diluents or excipients. Such compositions comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present disclosure are preferably formulated for intravenous administration.

Humoral immune responses, mediated primarily by helper T cells capable of activating B cells thus leading to antibody production, may be induced. A variety of techniques may be used for analyzing the type of immune responses induced by the compositions of the present disclosure, which are well disclosed in the art; e.g., Current Protocols in Immunology, Edited by: John E. Coligan, Ada M. Kruisbeek, David H. Margulies, Ethan M. Shevach, Warren Strober (2001) John Wiley & Sons, N.Y., N.Y.

In some aspects, provided herein is a method of treating cancer in an individual, the method comprising administering to the individual a therapeutically effective amount of any of the chimeric antigen receptors provided herein.

In some aspects, the cancer is a leukemia. In some aspects, the cancer is a CD94 expressing cancer.

In some aspects, the leukemia is T cell leukemia, T cell large granular leukemia, Natural Killer cell large granular leukemia, or Natural Killer cell leukemia.

In some aspects, the cancer is a lymphoma.

In some aspects, the lymphoma is T cell lymphoma, extranodal Natural Killer/T cell lymphoma, hepatosplenic T cell lymphoma, angioimmunoblastic T cell lymphoma, or anaplastic large cell lymphoma.

In some aspects, the cancer is lung cancer, bladder cancer, or melanoma.

In some aspects, provided herein is a method of treating or preventing graft-rejection of a transplant in a patient, the method comprising administering to the individual a therapeutically effective amount of any of the chimeric antigen receptors provided herein.

In some aspects, the transplant is an allogenic transplant.

In some aspects, the transplant is an organ transplant.

In some aspects, the transplant is a hematopoietic cell transplant.

In some aspects, the transplant is an induced pluripotent cell therapy.

In some aspects, provided herein is a method of modulating an immune response in a subject, the method comprising administering to the individual therapeutically effective amount of the antibody or antigen-binding fragment thereof of any of the chimeric antigen receptors provided herein.

In some aspects, the immune response is enhanced.

In some aspects, the immune response is mediated by Natural Killer cells and/or T cells.

The methods for administering the cell compositions disclosed herein includes any method which is effective to result in reintroduction of ex vivo genetically modified immune effector cells that either directly express a CAR of the disclosure in the subject or on reintroduction of the genetically modified progenitors of immune effector cells that on introduction into a subject differentiate into mature immune effector cells that express the CAR. One method comprises transducing peripheral blood T cells ex vivo with a nucleic acid construct in accordance with the disclosure and returning the transduced cells into the subject.

The present disclosure encompasses methods of preparing immune cells for immunotherapy comprising introducing, ex vivo, into such immune cells the polynucleotides or vectors encoding one of the CD94-specific chimeric antigen receptors disclosed herein.

The present disclosure also encompasses immune cells comprising a polynucleotide or lentiviral vector encoding one of the CD94-specific chimeric antigen receptors disclosed herein. In some aspects, these immune cells are used for immunotherapy (e.g., treatment of cancer).

The present disclosure also encompasses methods of genetically modifying immune cells to make them more suitable for allogeneic transplantation. According to a first aspect, the immune cell can be made allogeneic, for instance, by inactivating at least one gene expressing one or more component of T-cell receptor (TCR) as disclosed in WO 2013/176915, which can be combined with the inactivation of a gene encoding or regulating HLA or β2m protein expression. Accordingly, the risk of graft versus host syndrome and graft rejection is significantly reduced.

In some aspects, provided herein are immune cells comprising a chimeric antigen receptor of the disclosure. In some aspects, the immune cell is an immune effector cell. In some aspects, the immune cell is a T cell. In some aspects, the immune cell is a T lymphocyte selected from an inflammatory T lymphocyte, a cytotoxic T lymphocyte, a regulatory T lymphocyte, or a helper T lymphocyte. In some cases, the immune cell is a CD8+ cytotoxic T lymphocyte.

In some aspects, the engineered human T cell comprises a chimeric antigen receptor comprising, from N-terminus to C-terminus: (a) an extracellular ligand-binding domain comprising a scFv domain capable of binding to CD94; (b) a hinge; (c) a transmembrane domain; and (d) a cytoplasmic domain comprising a costimulatory domain and a signaling domain. In some aspects, the VH comprises a CDR H1 comprising the amino acid sequence set forth in SEQ ID NO: 3 or SEQ ID NO: 19, a CDR H2 comprising the amino acid sequence set forth in SEQ ID NO: 4 or SEQ ID NO: 20, a CDR H3 comprising the amino acid sequence set forth in SEQ ID NO: 5 or SEQ ID NO: 21. In some aspects, the VL comprises a CDR L1 comprising the amino acid sequence set forth in SEQ ID NO: 6 or SEQ ID NO: 22, a CDR L2 comprising the amino acid sequence YTS or the amino acid sequence set forth in SEQ ID NO: 23 or SEQ ID NO: 24, and a CDR L3 comprising the amino acid sequence set forth in SEQ ID NO: 8. In some aspects, the scFv domain comprises a VH and a VL.

In some aspects, the VH comprises a CDR H1 comprising the amino acid sequence set forth in SEQ ID NO: 3, a CDR H2 comprising the amino acid sequence set forth in SEQ ID NO: 4, and a CDR H3 comprising the amino acid sequence set forth in SEQ ID NO: 5. In some aspects, the VL comprises a CDR L1 comprising the amino acid sequence set forth in SEQ ID NO: 6, a CDR L2 comprising the amino acid sequence YTS, and a CDR L3 comprising the amino acid sequence set forth in SEQ ID NO: 8.

In some aspects, the VH comprises a CDR H1 comprising the amino acid sequence set forth in SEQ ID NO: 19, a CDR H2 comprising the amino acid sequence set forth in SEQ ID NO: 20, and a CDR H3 comprising the amino acid sequence set forth in SEQ ID NO: 21. In some aspects, the VL comprises a CDR L1 comprising the amino acid sequence set forth in SEQ ID NO: 22, a CDR L2 comprising the amino acid sequence set forth in SEQ ID NO: 23, and a CDR L3 comprising the amino acid sequence set forth in SEQ ID NO: 8. In some aspects, the VL comprises a CDR L1 comprising the amino acid sequence set forth in SEQ ID NO: 22, a CDR L2 comprising the amino acid sequence set forth in SEQ ID NO: 24, and a CDR L3 comprising the amino acid sequence set forth in SEQ ID NO: 8.

In some aspects, the engineered human T cell comprises a chimeric antigen receptor comprising an amino acid sequence having at least 85%, at least 90%, at least 91% at least 92%, at least 93%, at least 94%, at least 95%, at least 96%, at least 97%, at least 98%, at least 99%, or 100% sequence identity to SEQ ID NO: 9.

In some aspects, the engineered human T cell comprises a chimeric antigen receptor comprising an amino acid sequence having about 85%, about 90%, about 91% about 92%, about 93%, about 94%, about 95%, about 96%, about 97%, about 98%, about 99%, or about 100% sequence identity to SEQ ID NO: 9.

In some aspects, the engineered human T cell comprises a chimeric antigen receptor comprising an amino acid sequence having at least 85%, at least 90%, at least 91% at least 92%, at least 93%, at least 94%, at least 95%, at least 96%, at least 97%, at least 98%, at least 99%, or 100% sequence identity to SEQ ID NO: 56.

In some aspects, the engineered human T cell comprises a chimeric antigen receptor comprising an amino acid sequence having about 85%, about 90%, about 91% about 92%, about 93%, about 94%, about 95%, about 96%, about 97%, about 98%, about 99%, or about 100% sequence identity to SEQ ID NO: 56.

In some aspects, the engineered human T cell comprises a chimeric antigen receptor comprising an amino acid sequence having at least 85%, at least 90%, at least 91% at least 92%, at least 93%, at least 94%, at least 95%, at least 96%, at least 97%, at least 98%, at least 99%, or 100% sequence identity to SEQ ID NO: 57.

In some aspects, the engineered human T cell comprises a chimeric antigen receptor comprising an amino acid sequence having about 85%, about 90%, about 91% about 92%, about 93%, about 94%, about 95%, about 96%, about 97%, about 98%, about 99%, or about 100% sequence identity to SEQ ID NO: 57.

Whether prior to or after genetic modification of the engineered cells (e.g., T cells), even if the genetically modified immune cells of the present disclosure are activated and proliferate independently of antigen binding mechanisms, the immune cells, particularly T-cells of the present disclosure can be further activated and expanded generally using methods as disclosed, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005. T cells can be expanded in vitro or in vivo.

In some aspects, the T cells of the disclosure are expanded by contact with an agent that stimulates a CD3 TCR complex and a costimulatory molecule on the surface of the T cells to create an activation signal for the T-cell. For example, chemicals such as calcium ionophore A23187, phorbol 12-myristate 13-acetate (PMA), or mitogenic lectins like phytohemagglutinin (PHA) can be used to create an activation signal for the T-cell.

As non-limiting examples, T cell populations can be stimulated in vitro such as by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For costimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. Conditions appropriate for T cell culture include an appropriate media (e.g., Minimal Essential Media or RPMI Media 1640 or, X-vivo 5, (Lonza)) that can contain factors necessary for proliferation and viability, including serum (e.g., fetal bovine or human serum), interleukin-2 (IL-2), insulin, IFN-g, IL-4, IL-7, GM-CSF, IL-10, IL-2, IL-15, TGFp, and TNF-α or any other additives for the growth of cells known to the skilled artisan. Other additives for the growth of cells include, but are not limited to, surfactant, plasmanate, and reducing agents such as N-acetyl-cysteine and 2-mercaptoethanol. Media can include RPMI 1640, A1M-V, DMEM, MEM, a-MEM, F-12, X-Vivo 1, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% O2). T cells that have been exposed to varied stimulation times can exhibit different characteristics.

In some aspects, the cells can be expanded by co-culturing with tissue or cells. The cells can also be expanded in vivo, for example in the subject's blood after administrating said cell into the subject.

### IV. Antibody Production

Antibodies and antigen-binding fragments thereof that immunospecifically bind to CD94 (e.g., human CD94) can be produced by any method known in the art for the synthesis of antibodies and antigen-binding fragments thereof, for example, by chemical synthesis or by recombinant expression techniques. The methods disclosed herein employ, unless otherwise indicated, conventional techniques in molecular biology, microbiology, genetic analysis, recombinant DNA, organic chemistry, biochemistry, PCR, oligonucleotide synthesis and modification, nucleic acid hybridization, and related fields within the skill of the art. These techniques are disclosed, for example, in the references cited herein and are fully explained in the literature. See, e.g., Sambrook J et al., (2001) Molecular Cloning: A Laboratory Manual, Cold Spring Harbor Laboratory Press, Cold Spring Harbor, NY; Ausubel F M et al., Current Protocols in Molecular Biology, John Wiley & Sons (1987 and annual updates); Current Protocols in Immunology, John Wiley & Sons (1987 and annual updates) Gait (ed.) (1984) Oligonucleotide Synthesis: A Practical Approach, IRL Press; Eckstein (ed.) (1991) Oligonucleotides and Analogues: A Practical Approach, IRL Press; Birren B et al., (eds.) (1999) Genome Analysis: A Laboratory Manual, Cold Spring Harbor Laboratory Press.

In some aspects, provided herein is a method of making an antibody or antigen-binding fragment which immunospecifically binds to CD94 (e.g., human CD94) comprising culturing a cell or host cell disclosed herein. In some aspects, provided herein is a method of making an antibody or antigen-binding fragment thereof which immunospecifically binds to CD94 (e.g., human CD94) comprising expressing (e.g., recombinantly expressing) the antibody or antigen-binding fragment thereof using a cell or host cell disclosed herein (e.g., a cell or a host cell comprising polynucleotides encoding an antibody or antigen-binding fragment thereof disclosed herein). In some aspects, the cell is an isolated cell. In some aspects, the exogenous polynucleotides have been introduced into the cell. In some aspects, the method further comprises the step of purifying the antibody or antigen-binding fragment obtained from the cell or host cell.

Methods for producing polyclonal antibodies are known in the art (see, for example, Chapter 11 in: Short Protocols in Molecular Biology, (2002) 5th Ed., Ausubel F M et al., eds., John Wiley and Sons, New York).

Monoclonal antibodies or antigen-binding fragments thereof can be prepared using a wide variety of techniques known in the art including the use of hybridoma, recombinant, and phage display technologies, yeast-based presentation technologies, or a combination thereof. For example, monoclonal antibodies or antigen-binding fragments thereof can be produced using hybridoma techniques including those known in the art and taught, for example, in Harlow E & Lane D, Antibodies: A Laboratory Manual, (Cold Spring Harbor Laboratory Press, 2nd ed. 1988); Hammerling G J et al., in: Monoclonal Antibodies and T-Cell Hybridomas 563 681 (Elsevier, N.Y., 1981), or as disclosed in Kohler G & Milstein C (1975) Nature 256:495. Examples of yeast-based presentation methods that can be employed to select and generate the antibodies disclosed herein include those disclosed in, for example, WO2009/036379A2; WO2010/105256; and WO2012/009568, each of which is herein incorporated by reference in its entirety.

In specific aspects, a monoclonal antibody or antigen-binding fragment thereof can be produced using the hybridoma method first disclosed by Kohler et al., Nature, 256:495 (1975), as mentioned above. In the hybridoma method, a mouse or another appropriate host animal is immunized as above disclosed to elicit lymphocytes that produce or are capable of producing antibodies that will specifically bind to the protein used for immunization, for example, variant mixtures of CD94. Lymphocytes then are fused with myeloma cells using a suitable fusing agent, such as polyethylene glycol, to form a hybridoma cell (Goding, Monoclonal Antibodies: Principles and Practice, pp. 59-103, Academic Press, 1986).

In some aspects, a monoclonal antibody or antigen-binding fragment thereof is an antibody or antigen-binding fragment produced by a clonal cell (e.g., hybridoma or host cell producing a recombinant antibody or antigen-binding fragment), wherein the antibody or antigen-binding fragment immunospecifically binds to CD94 (e.g., human CD94) as determined, e.g., by ELISA or other antigen-binding assays known in the art or in the Examples provided herein. In some aspects, a monoclonal antibody or antigen-binding fragment thereof can be a rodent or murine antibody or antigen-binding fragment thereof. In some aspects, a monoclonal antibody or antigen-binding fragment thereof can be a chimeric or a humanized antibody or antigen-binding fragment thereof. In some aspects, a monoclonal antibody or antigen-binding fragment thereof can be a Fab fragment or an F(ab′)2 fragment. Monoclonal antibodies or antigen-binding fragments thereof disclosed herein can, for example, be made by the hybridoma method as disclosed in Kohler G & Milstein C (1975) Nature 256:495 or can, e.g., be isolated from phage libraries using the techniques as disclosed herein, for example. Other methods for the preparation of clonal cell lines and of monoclonal antibodies and antigen-binding fragments thereof expressed thereby are well known in the art (see, for example, Chapter 11 in: Short Protocols in Molecular Biology, (2002) 5th Ed., Ausubel F M et al.).

Antigen-binding fragments of antibodies disclosed herein can be generated by any technique known to those of skill in the art. For example, Fab and F(ab′)2 fragments disclosed herein can be produced by proteolytic cleavage of immunoglobulin molecules, using enzymes such as papain (to produce Fab fragments) or pepsin (to produce F(ab′)2 fragments). A Fab fragment corresponds to one of the two identical arms of a tetrameric antibody molecule and contains the complete light chain paired with the VH and CHI domains of the heavy chain. An F(ab′)2 fragment contains the two antigen-binding arms of a tetrameric antibody molecule linked by disulfide bonds in the hinge region.

Further, the antibodies or antigen-binding fragments thereof disclosed herein can also be generated using various phage display and/or yeast-based presentation methods known in the art. In phage display methods, proteins are displayed on the surface of phage particles which carry the polynucleotide sequences encoding them. In particular, DNA sequences encoding VH and VL domains are amplified from animal cDNA libraries (e.g., human or murine cDNA libraries of affected tissues). The DNA encoding the VH and VL domains are recombined together with a scFv linker by PCR and cloned into a phagemid vector. The vector is electroporated in E. coli and the E. coli is infected with helper phage. Phage used in these methods are typically filamentous phage including fd and M13, and the VH and VL domains are usually recombinantly fused to either the phage gene III or gene VIII. Phage expressing an antibody or antigen-binding fragment thereof that binds to a particular antigen can be selected or identified with antigen, e.g., using labeled antigen or antigen bound or captured to a solid surface or bead. Examples of phage display methods that can be used to make the antibodies or fragments disclosed herein include those disclosed in Brinkman U et al., (1995) J Immunol Methods 182:41-50; Ames R S et al., (1995) J Immunol Methods 184:177-186; Kettleborough C A et al., (1994) Eur J Immunol 24:952-958; Persic L et al., (1997) Gene 187:9-18; Burton D R & Barbas C F (1994) Advan Immunol 57:191-280; PCT Application No. PCT/GB91/001134; International Publication Nos. WO 90/02809, WO 91/10737, WO 92/01047, WO 92/18619, WO 93/1 1236, WO 95/15982, WO 95/20401, and WO 97/13844; and U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727, 5,733,743, and 5,969,108.

An antibody or antigen-binding fragment thereof can be selected from any class of immunoglobulins, including IgM, IgG, IgD, IgA, and IgE, and any isotype, including IgG1, IgG2, IgG3 and IgG4.

a. Polynucleotides

In some aspects, provided herein are polynucleotides comprising a nucleotide sequence encoding an antibody or antigen-binding fragment thereof disclosed herein or a domain thereof (e.g., a variable light chain region and/or variable heavy chain region) that immunospecifically binds to a CD94 (e.g., human CD94) antigen, and vectors, e.g., vectors comprising such polynucleotides for recombinant expression in host cells (e.g., E. coli and mammalian cells).

In some aspects, provided herein are polynucleotides comprising a nucleotide sequence encoding the chimeric antigen receptors (CARs) disclosed herein.

In some aspects, provided herein are polynucleotides comprising nucleotide sequences encoding antibodies or antigen-binding fragments thereof, which immunospecifically bind to a CD94 polypeptide (e.g., human CD94) and comprise an amino acid sequence as disclosed herein.

In some aspects, an antibody or antigen-binding fragment thereof provided herein binds to CD94 and comprises six CDRs (e.g., a CDR H1, a CDR H2, a CDR H3, a CDR L1, a CDR L2, and a CDR L3). The CDRs may be determined by a number of algorithms in the art, such as IMGT (Table 6), abYsis (Table 7), and Kabat (Table 8).

For example, the international ImMunoGeneTics information System® (http://www.imgt.org) is based on the IMGT numbering for all immunoglobulin and T cell receptor V-REGIONs of all species (IMGT®, the international ImMunoGeneTics information System®; Lefranc M P et al., (1991) Nucleic Acids Res. 27 (1): 209-12; Ruiz M et al., (2000) Nucleic Acids Res. 28 (1): 219-21; Lefranc M P (2001) Nucleic Acids Res. 29 (1): 207-9; Lefranc M P (2003) Nucleic Acids Res. 31 (1): 307-10; Lefranc M P et al., (2005) Dev. Comp. Immunol. 29 (3): 185-203; Kaas Q et al., (2007) Briefings in Functional Genomics & Proteomics, 6 (4): 253-64).

The abYsis algorithm compiles antibody protein sequences from EMBLIG which contains antibody information extracted from the EMBL-ENA databank, the Kabat collection, and the Protein Databank (Swindells M B et al., (2017) J Mol Biol. 2017 Feb. 3; 429 (3): 356-364).

The Kabat definition is based on sequence variability and is the most commonly used (Kabat E A et al., supra).

TABLE 6
IMGT CDR Nucleotide Sequences

| CDR | Algorithm | Sequence | SEQ ID NO |
|---|---|---|---|
| CDR H1 | IMGT | GGATTTAACATCAAGGACACATAT | 12 |
| CDR H2 | IMGT | ATCGACCCTGCCAACGGCAATACT | 13 |
| CDR H3 | IMGT | GTGTACTACTGCGTGTACGGCAGA | 14 |
| | | GGCTCTGCCTGGTTCGCCTTT | |
| CDR L1 | IMGT | CAGGACATCAGCAACTAC | 15 |
| CDR L2 | IMGT | TACACCAGC | — |
| CDR L3 | IMGT | CAGCAGGGCAACACACTGCCTTGGACC | 17 |

TABLE 7
abYsis CDR Nucleotide Sequences

| | | | SEQ ID |
|---|---|---|---|
| CDR | Algorithm | Sequence | NO |
| CDR H1 | ab Ysis | GACACATATATGCAC | 25 |
| CDR H2 | ab Ysis | CGGATCGACCCTGCCAACGGCAA | 26 |
| | | TACTCAATACGACCCCAAGTTCCAGGGC | |
| CDR H3 | ab Ysis | TGCGTGTACGGCAGAGGCTCTGCC | 27 |
| | | TGGTTCGCCTTT | |
| CDR L1 | ab Ysis | AGAGCCAGCCAGGACATCAGCAACTA | 28 |
| | | CCTGAAC | |
| CDR L2 | ab Ysis | TACACCAGCCGGCTGCACAGC | 29 |
| CDR L3 | ab Ysis | CAGCAGGGCAACACACTGCCTTGGACC | 17 |

TABLE 8
Kabat CDR Nucleotide Sequences

| | | | SEQ ID |
|---|---|---|---|
| CDR | Algorithm | Sequence | NO |
| CDR H1 | Kabat | GACACATATATGCAC | 25 |
| CDR H2 | Kabat | CGGATCGACCCTGCCAACGGCAA | 26 |
| | | TACTCAATACGACCCCAAGTTCCAGGGC | |
| CDR H3 | Kabat | TACTGCGTGTACGGCAGAGGCTCTGCC | 27 |
| | | TGGTTCGCCTTT | |
| CDR L1 | Kabat | AGAGCCAGCCAGGACATCAGCAACTA | 28 |
| | | CCTGAA | |
| CDR L2 | Kabat | TACACCAGCCGGCTGCAC | 30 |
| CDR L3 | Kabat | CAGCAGGGCAACACACTGCCTTGGACC | 17 |

In some aspects, disclosed herein is an isolated polynucleotide comprises a nucleic acid molecule encoding any of the VH or heavy chain of the anti-CD94 antibody or antigen-binding fragment thereof disclosed herein. In some aspects, the VH is encoded by a polynucleotide sequence comprising a sequence corresponding to the polynucleotide sequence of SEQ ID NO: 10.

In some aspects, disclosed herein is an isolated polynucleotide comprising a nucleic acid molecule encoding any of the VL or light chain of the anti-CD94 antibody or antigen-binding fragment thereof disclosed herein. In some aspects, the VL is encoded by a polynucleotide sequence comprising a sequence corresponding to the polynucleotide sequence of SEQ ID NO: 11.

In some aspects, provided herein is an antibody or antigen-binding fragment thereof capable of binding to CD94, wherein the antibody or antigen-binding fragment thereof comprises a complementary determining region (CDR) H1 comprising the polynucleotide sequence set forth in SEQ ID NO: 12 or SEQ ID NO: 25, a CDR H2 comprising the polynucleotide sequence set forth in SEQ ID NO: 13 or SEQ ID NO: 26, a CDR H3 comprising the polynucleotide sequence set forth in SEQ ID NO: 14 or SEQ ID NO: 27, a CDR L1 comprising the polynucleotide sequence set forth in SEQ ID NO: 15 or SEQ ID NO: 28, a CDR L2 comprising the polynucleotide sequence TACACCAGC or the polynucleotide sequence set forth in SEQ ID NO: 29 or SEQ ID NO: 30, and a CDR L3 comprising the polynucleotide sequence set forth in SEQ ID NO: 17.

In some aspects, provided herein is an antibody or antigen-binding fragment thereof capable of binding to CD94, wherein the antibody or antigen-binding fragment thereof comprises a complementary determining region (CDR) H1 comprising the polynucleotide sequence set forth in SEQ ID NO: 12, a CDR H2 comprising the polynucleotide sequence set forth in SEQ ID NO: 13, a CDR H3 comprising the polynucleotide sequence set forth in SEQ ID NO: 14, a CDR L1 comprising the polynucleotide sequence set forth in SEQ ID NO: 15, a CDR L2 comprising the polynucleotide sequence TACACCAGC, and a CDR L3 comprising the polynucleotide sequence set forth in SEQ ID NO: 17.

In some aspects, provided herein is an antibody or antigen-binding fragment thereof capable of binding to CD94, wherein the antibody or antigen-binding fragment thereof comprises a complementary determining region (CDR) H1 comprising the polynucleotide sequence set forth in SEQ ID NO: 25, a CDR H2 comprising the polynucleotide sequence set forth in SEQ ID NO: 26, a CDR H3 comprising the polynucleotide sequence set forth in SEQ ID NO: 27, a CDR L1 comprising the polynucleotide sequence set forth in SEQ ID NO: 28, a CDR L2 comprising the polynucleotide sequence set forth in SEQ ID NO: 29, and a CDR L3 comprising the polynucleotide sequence set forth in SEQ ID NO: 17.

In some aspects, provided herein is an antibody or antigen-binding fragment thereof capable of binding to CD94, wherein the antibody or antigen-binding fragment thereof comprises a complementary determining region (CDR) H1 comprising the polynucleotide sequence set forth in SEQ ID NO: 25, a CDR H2 comprising the polynucleotide sequence set forth in SEQ ID NO: 26, a CDR H3 comprising the polynucleotide sequence set forth in SEQ ID NO: 27, a CDR L1 comprising the polynucleotide sequence set forth in SEQ ID NO: 28, a CDR L2 comprising the polynucleotide sequence set forth in SEQ ID NO: 30, and a CDR L3 comprising the polynucleotide sequence set forth in SEQ ID NO: 17.

In some aspects, the antibody or antigen-binding fragment thereof capable of binding CD94 comprises a heavy chain variable region (VH) and a light chain variable region (VL). In some aspects, the VH comprises a polynucleotide sequence having at least 85%, at least 90%, at least 91% at least 92%, at least 93%, at least 94%, at least 95%, at least 96%, at least 97%, at least 98%, at least 99%, or 100% sequence identity to SEQ ID NO: 10. In some aspects, the VL comprises a polynucleotide sequence having at least 85%, at least 90%, at least 91%, at least 92%, at least 93%, at least 94%, at least 95%, at least 96%, at least 97%, at least 98%, at least 99%, or 100% sequence identity to SEQ ID NO: 11.

In some aspects, the antibody or antigen-binding fragment thereof capable of binding CD94 comprises a heavy chain variable region (VH) and a light chain variable region (VL). In some aspects, the VH comprises a polynucleotide sequence having about 85%, about 90%, about 91% about 92%, about 93%, about 94%, about 95%, about 96%, about 97%, about 98%, about 99%, or about 100% sequence identity to SEQ ID NO: 10. In some aspects, the VL comprises a polynucleotide sequence having about 85%, about 90%, about 91%, about 92%, about 93%, about 94%, about 95%, about 96%, about 97%, about 98%, about 99%, or about 100% sequence identity to SEQ ID NO: 11.

In some aspects, the antibody or antigen-binding fragment comprises a VH comprising a polynucleotide sequence at least 85% identical to the polynucleotide sequence SEQ ID NO: 10. In some aspects, the antibody or antigen-binding fragment comprises a VH comprising a polynucleotide sequence at least 90% identical to the polynucleotide sequence SEQ ID NO: 10. In some aspects, the antibody or antigen-binding fragment comprises a VH comprising a polynucleotide sequence at least 95% identical to the polynucleotide sequence SEQ ID NO: 10. In some aspects, the antibody or antigen-binding fragment comprises a VH comprising a polynucleotide sequence at least 98% identical to the polynucleotide sequence SEQ ID NO: 10. In some aspects, the antibody or antigen-binding fragment comprises a VH comprising a polynucleotide sequence at least 99% identical to the polynucleotide sequence SEQ ID NO: 10. In some aspects, the antibody or antigen-binding fragment comprises a VH comprising a polynucleotide sequence corresponding to the polynucleotide sequence of SEQ ID NO: 10.

In some aspects, the antibody or antigen-binding fragment comprises a VL comprising a polynucleotide sequence at least 85% identical to the polynucleotide sequence of SEQ ID NO: 11. In some aspects, the antibody or antigen-binding fragment comprises a VL comprising a polynucleotide sequence at least 90% identical to the polynucleotide sequence of SEQ ID NO: 11. In some aspects, the antibody or antigen-binding fragment comprises a VL comprising a polynucleotide sequence at least 95% identical to the polynucleotide sequence of SEQ ID NO: 11. In some aspects, the antibody or antigen-binding fragment comprises a VL comprising a polynucleotide sequence at least 98% identical to the polynucleotide sequence of SEQ ID NO: 11. In some aspects, the antibody or antigen-binding fragment comprises a VL comprising a polynucleotide sequence at least 99% identical to the polynucleotide sequence of SEQ ID NO: 11. In some aspects, the antibody or antigen-binding fragment comprises a VL comprising a polynucleotide sequence corresponding to the polynucleotide sequence of SEQ ID NO: 11.

In some aspects, the antibody or antigen-binding fragment comprises a VH comprising a polynucleotide sequence about 85% identical to the polynucleotide sequence SEQ ID NO: 10. In some aspects, the antibody or antigen-binding fragment comprises a VH comprising a polynucleotide sequence about 90% identical to the polynucleotide sequence SEQ ID NO: 10. In some aspects, the antibody or antigen-binding fragment comprises a VH comprising a polynucleotide sequence about 95% identical to the polynucleotide sequence SEQ ID NO: 10. In some aspects, the antibody or antigen-binding fragment comprises a VH comprising a polynucleotide sequence about 98% identical to the polynucleotide sequence SEQ ID NO: 10. In some aspects, the antibody or antigen-binding fragment comprises a VH comprising a polynucleotide sequence about 99% identical to the polynucleotide sequence SEQ ID NO: 10.

In some aspects, the antibody or antigen-binding fragment comprises a VL comprising a polynucleotide sequence about 85% identical to the polynucleotide sequence of SEQ ID NO: 11. In some aspects, the antibody or antigen-binding fragment comprises a VL comprising a polynucleotide sequence about 90% identical to the polynucleotide sequence of SEQ ID NO: 11. In some aspects, the antibody or antigen-binding fragment comprises a VL comprising a polynucleotide sequence about 95% identical to the polynucleotide sequence of SEQ ID NO: 11. In some aspects, the antibody or antigen-binding fragment comprises a VL comprising a polynucleotide sequence about 98% identical to the polynucleotide sequence of SEQ ID NO: 11. In some aspects, the antibody or antigen-binding fragment comprises a VL comprising a polynucleotide sequence about 99% identical to the polynucleotide sequence of SEQ ID NO: 11.

In some aspects, disclosed herein is an isolated polynucleotide comprising a nucleic acid molecule encoding any of the chimeric antigen receptors (CARs) disclosed herein. In some aspects, the chimeric antigen receptor comprises a polynucleotide sequence having about 85%, about 90%, about 91%, about 92%, about 93%, about 94%, about 95%, about 96%, about 97%, about 98%, about 99%, or about 100% sequence identity to a sequence corresponding to the polynucleotide sequence of SEQ ID NO: 18. In some aspects, the chimeric antigen receptor comprises a polynucleotide sequence having at least 85%, at least 90%, at least 91%, at least 92%, at least 93%, at least 94%, at least 95%, at least 96%, at least 97%, at least 98%, at least 99%, or 100% sequence identity to a sequence corresponding to the polynucleotide sequence of SEQ ID NO: 18. In some aspects, the chimeric antigen receptor comprises a polynucleotide sequence corresponding to the polynucleotide sequence of SEQ ID NO: 18.

In some aspects, the chimeric antigen receptor comprises a polynucleotide sequence having about 85%, about 90%, about 91%, about 92%, about 93%, about 94%, about 95%, about 96%, about 97%, about 98%, about 99%, or about 100% sequence identity to a sequence corresponding to the polynucleotide sequence of SEQ ID NO: 58. In some aspects, the chimeric antigen receptor comprises a polynucleotide sequence having at least 85%, at least 90%, at least 91%, at least 92%, at least 93%, at least 94%, at least 95%, at least 96%, at least 97%, at least 98%, at least 99%, or 100% sequence identity to a sequence corresponding to the polynucleotide sequence of SEQ ID NO: 58. In some aspects, the chimeric antigen receptor comprises a polynucleotide sequence corresponding to the polynucleotide sequence of SEQ ID NO: 58.

In some aspects, the chimeric antigen receptor comprises a polynucleotide sequence having about 85%, about 90%, about 91%, about 92%, about 93%, about 94%, about 95%, about 96%, about 97%, about 98%, about 99%, or about 100% sequence identity to a sequence corresponding to the polynucleotide sequence of SEQ ID NO: 59. In some aspects, the chimeric antigen receptor comprises a polynucleotide sequence having at least 85%, at least 90%, at least 91%, at least 92%, at least 93%, at least 94%, at least 95%, at least 96%, at least 97%, at least 98%, at least 99%, or 100% sequence identity to a sequence corresponding to the polynucleotide sequence of SEQ ID NO: 59. In some aspects, the chimeric antigen receptor comprises a polynucleotide sequence corresponding to the polynucleotide sequence of SEQ ID NO: 59.

In some aspects, the chimeric antigen receptor comprises from N-terminus to C-terminus: (a) an extracellular ligand-binding domain comprising a scFv domain capable of binding to CD94; (b) a hinge; (c) a transmembrane domain; and (d) a cytoplasmic domain comprising a costimulatory domain and a signaling domain. In some aspects, the signal peptide comprises a CD8α signal peptide. In some aspects, the scFv comprises a linker. In some aspects, the hinge comprises a CD8α hinge domain. In some aspects, the transmembrane domain comprises a CD8α transmembrane domain. In some aspects, the costimulatory domain comprises a 4-1BB costimulatory domain. In some aspects, the signaling domain comprises a CD3zeta signaling domain.

In some aspects, the CD8α signal peptide comprises a polynucleotide sequence having about 85%, about 90%, about 91% about 92%, about 93%, about 94%, about 95%, about 96%, about 97%, about 98%, about 99%, or about 100% sequence identity to SEQ ID NO: 39. In some aspects, the CD8α signal peptide comprises a polynucleotide sequence having at least about 85%, at least about 90%, at least about 91%, at least about 92%, at least about 93%, at least about 94%, at least about 95%, at least about 96%, at least about 97%, at least about 98%, at least about 99%, or 100% sequence identity to SEQ ID NO: 39.

In some aspects, the CD8α signal peptide comprises a polynucleotide sequence at least 85% identical to the polynucleotide sequence of SEQ ID NO: 39. In some aspects, the CD8α signal peptide comprises a polynucleotide sequence at least 90% identical to the polynucleotide sequence of SEQ ID NO: 39. In some aspects, the CD8α signal peptide comprises a polynucleotide sequence at least 95% identical to the polynucleotide sequence of SEQ ID NO: 39. In some aspects, the CD8α signal peptide comprises a polynucleotide sequence at least 98% identical to the polynucleotide sequence of SEQ ID NO: 39. In some aspects, the CD8α signal peptide comprises a polynucleotide sequence at least 99% identical to the polynucleotide sequence of SEQ ID NO: 39. In some aspects, the CD8α signal peptide comprises a polynucleotide sequence corresponding to the polynucleotide sequence of SEQ ID NO: 39.

In some aspects, the CD8α signal peptide comprises a polynucleotide sequence about 85% identical to the polynucleotide sequence SEQ ID NO: 39. In some aspects, the CD8α signal peptide comprises a polynucleotide sequence about 90% identical to the polynucleotide sequence SEQ ID NO: 39. In some aspects, the CD8α signal peptide comprises a polynucleotide sequence about 95% identical to the polynucleotide sequence SEQ ID NO: 39. In some aspects, the CD8α signal peptide comprises a polynucleotide sequence about 98% identical to the polynucleotide sequence SEQ ID NO: 39. In some aspects, the CD8α signal peptide comprises a polynucleotide sequence about 99% identical to the polynucleotide sequence SEQ ID NO: 39.

In some aspects, the scFv comprises a polynucleotide sequence having about 85%, about 90%, about 91% about 92%, about 93%, about 94%, about 95%, about 96%, about 97%, about 98%, about 99%, or about 100% sequence identity to SEQ ID NO: 40. In some aspects, the scFv comprises a polynucleotide sequence having at least about 85%, at least about 90%, at least about 91%, at least about 92%, at least about 93%, at least about 94%, at least about 95%, at least about 96%, at least about 97%, at least about 98%, at least about 99%, or 100% sequence identity to SEQ ID NO: 40.

In some aspects, the scFv comprises a polynucleotide sequence at least 85% identical to the polynucleotide sequence of SEQ ID NO: 40. In some aspects, the scFv comprises a polynucleotide sequence at least 90% identical to the polynucleotide sequence of SEQ ID NO: 40. In some aspects, the scFv comprises a polynucleotide sequence at least 95% identical to the polynucleotide sequence of SEQ ID NO: 40. In some aspects, the scFv comprises a polynucleotide sequence at least 98% identical to the polynucleotide sequence of SEQ ID NO: 40. In some aspects, the scFv comprises a polynucleotide sequence at least 99% identical to the polynucleotide sequence of SEQ ID NO: 40. In some aspects, the scFv comprises a polynucleotide sequence corresponding to the polynucleotide sequence of SEQ ID NO: 40.

In some aspects, the scFv comprises a polynucleotide sequence about 85% identical to the polynucleotide sequence SEQ ID NO: 40. In some aspects, the scFv comprises a polynucleotide sequence about 90% identical to the polynucleotide sequence SEQ ID NO: 40. In some aspects, the scFv comprises a polynucleotide sequence about 95% identical to the polynucleotide sequence SEQ ID NO: 40. In some aspects, the scFv comprises a polynucleotide sequence about 98% identical to the polynucleotide sequence SEQ ID NO: 40. In some aspects, the scFv comprises a polynucleotide sequence about 99% identical to the polynucleotide sequence SEQ ID NO: 40.

In some aspects, the linker comprises a polynucleotide sequence having about 85%, about 90%, about 91% about 92%, about 93%, about 94%, about 95%, about 96%, about 97%, about 98%, about 99%, or about 100% sequence identity to SEQ ID NO: 41. In some aspects, the linker comprises a polynucleotide sequence having at least about 85%, at least about 90%, at least about 91%, at least about 92%, at least about 93%, at least about 94%, at least about 95%, at least about 96%, at least about 97%, at least about 98%, at least about 99%, or 100% sequence identity to SEQ ID NO: 41.

In some aspects, the linker comprises a polynucleotide sequence at least 85% identical to the polynucleotide sequence of SEQ ID NO: 41. In some aspects, the linker comprises a polynucleotide sequence at least 90% identical to the polynucleotide sequence of SEQ ID NO: 41. In some aspects, the linker comprises a polynucleotide sequence at least 95% identical to the polynucleotide sequence of SEQ ID NO: 41. In some aspects, the linker comprises a polynucleotide sequence at least 98% identical to the polynucleotide sequence of SEQ ID NO: 41. In some aspects, the linker comprises a polynucleotide sequence at least 99% identical to the polynucleotide sequence of SEQ ID NO: 41. In some aspects, the linker comprises a polynucleotide sequence corresponding to the polynucleotide sequence of SEQ ID NO: 41.

In some aspects, the linker comprises a polynucleotide sequence about 85% identical to the polynucleotide sequence SEQ ID NO: 41. In some aspects, the linker comprises a polynucleotide sequence about 90% identical to the polynucleotide sequence SEQ ID NO: 41. In some aspects, the linker comprises a polynucleotide sequence about 95% identical to the polynucleotide sequence SEQ ID NO: 41. In some aspects, the linker comprises a polynucleotide sequence about 98% identical to the polynucleotide sequence SEQ ID NO: 41. In some aspects, the linker comprises a polynucleotide sequence about 99% identical to the polynucleotide sequence SEQ ID NO: 41.

In some aspects, the CD8α hinge domain comprises a polynucleotide sequence having about 85%, about 90%, about 91% about 92%, about 93%, about 94%, about 95%, about 96%, about 97%, about 98%, about 99%, or about 100% sequence identity to SEQ ID NO: 42. In some aspects, the CD8α hinge domain comprises a polynucleotide sequence having at least about 85%, at least about 90%, at least about 91%, at least about 92%, at least about 93%, at least about 94%, at least about 95%, at least about 96%, at least about 97%, at least about 98%, at least about 99%, or 100% sequence identity to SEQ ID NO: 42.

In some aspects, the CD8α hinge domain comprises a polynucleotide sequence at least 85% identical to the polynucleotide sequence of SEQ ID NO: 42. In some aspects, the CD8α hinge domain comprises a polynucleotide sequence at least 90% identical to the polynucleotide sequence of SEQ ID NO: 42. In some aspects, the CD8α hinge domain comprises a polynucleotide sequence at least 95% identical to the polynucleotide sequence of SEQ ID NO: 42. In some aspects, the CD8α hinge domain comprises a polynucleotide sequence at least 98% identical to the polynucleotide sequence of SEQ ID NO: 42. In some aspects, the CD8α hinge domain comprises a polynucleotide sequence at least 99% identical to the polynucleotide sequence of SEQ ID NO: 42. In some aspects, the CD8α hinge domain comprises a polynucleotide sequence corresponding to the polynucleotide sequence of SEQ ID NO: 42.

In some aspects, the CD8α hinge domain comprises a polynucleotide sequence about 85% identical to the polynucleotide sequence SEQ ID NO: 42. In some aspects, the CD8α hinge domain comprises a polynucleotide sequence about 90% identical to the polynucleotide sequence SEQ ID NO: 42. In some aspects, the CD8 hinge domain comprises a polynucleotide sequence about 95% identical to the polynucleotide sequence SEQ ID NO: 42. In some aspects, the CD8α hinge domain comprises a polynucleotide sequence about 98% identical to the polynucleotide sequence SEQ ID NO: 42. In some aspects, the CD8α hinge domain comprises a polynucleotide sequence about 99% identical to the polynucleotide sequence SEQ ID NO: 42.

In some aspects, the CD8α transmembrane domain comprises a polynucleotide sequence having about 85%, about 90%, about 91% about 92%, about 93%, about 94%, about 95%, about 96%, about 97%, about 98%, about 99%, or about 100% sequence identity to SEQ ID NO: 43. In some aspects, the CD8α transmembrane domain comprises a polynucleotide sequence having at least about 85%, at least about 90%, at least about 91%, at least about 92%, at least about 93%, at least about 94%, at least about 95%, at least about 96%, at least about 97%, at least about 98%, at least about 99%, or 100% sequence identity to SEQ ID NO: 43.

In some aspects, the CD8α transmembrane domain comprises a polynucleotide sequence at least 85% identical to the polynucleotide sequence of SEQ ID NO: 43. In some aspects, the CD8α transmembrane domain comprises a polynucleotide sequence at least 90% identical to the polynucleotide sequence of SEQ ID NO: 43. In some aspects, the CD8α transmembrane domain comprises a polynucleotide sequence at least 95% identical to the polynucleotide sequence of SEQ ID NO: 43. In some aspects, the CD8α transmembrane domain comprises a polynucleotide sequence at least 98% identical to the polynucleotide sequence of SEQ ID NO: 43. In some aspects, the CD8α transmembrane domain comprises a polynucleotide sequence at least 99% identical to the polynucleotide sequence of SEQ ID NO: 43. In some aspects, the CD8α transmembrane domain comprises a polynucleotide sequence corresponding to the polynucleotide sequence of SEQ ID NO: 43.

In some aspects, the CD8α transmembrane domain comprises a polynucleotide sequence about 85% identical to the polynucleotide sequence SEQ ID NO: 43. In some aspects, the CD8α transmembrane domain comprises a polynucleotide sequence about 90% identical to the polynucleotide sequence SEQ ID NO: 43. In some aspects, the CD8α transmembrane domain comprises a polynucleotide sequence about 95% identical to the polynucleotide sequence SEQ ID NO: 43. In some aspects, the CD8α transmembrane domain comprises a polynucleotide sequence about 98% identical to the polynucleotide sequence SEQ ID NO: 43. In some aspects, the CD8α transmembrane domain comprises a polynucleotide sequence about 99% identical to the polynucleotide sequence SEQ ID NO: 43.

In some aspects, the 4-1BB costimulatory domain comprises a polynucleotide sequence having about 85%, about 90%, about 91% about 92%, about 93%, about 94%, about 95%, about 96%, about 97%, about 98%, about 99%, or about 100% sequence identity to SEQ ID NO: 44. In some aspects, the 4-1BB costimulatory domain comprises a polynucleotide sequence having at least about 85%, at least about 90%, at least about 91%, at least about 92%, at least about 93%, at least about 94%, at least about 95%, at least about 96%, at least about 97%, at least about 98%, at least about 99%, or 100% sequence identity to SEQ ID NO: 44.

In some aspects, the 4-1BB costimulatory domain comprises a polynucleotide sequence at least 85% identical to the polynucleotide sequence of SEQ ID NO: 44. In some aspects, the 4-1BB costimulatory domain comprises a polynucleotide sequence at least 90% identical to the polynucleotide sequence of SEQ ID NO: 44. In some aspects, the 4-1BB costimulatory domain comprises a polynucleotide sequence at least 95% identical to the polynucleotide sequence of SEQ ID NO: 44. In some aspects, the 4-1BB costimulatory domain comprises a polynucleotide sequence at least 98% identical to the polynucleotide sequence of SEQ ID NO: 44. In some aspects, the 4-1BB costimulatory domain comprises a polynucleotide sequence at least 99% identical to the polynucleotide sequence of SEQ ID NO: 44. In some aspects, the 4-1BB costimulatory domain comprises a polynucleotide sequence corresponding to the polynucleotide sequence of SEQ ID NO: 44.

In some aspects, the 4-1BB costimulatory domain comprises a polynucleotide sequence about 85% identical to the polynucleotide sequence SEQ ID NO: 44. In some aspects, the 4-1BB costimulatory domain comprises a polynucleotide sequence about 90% identical to the polynucleotide sequence SEQ ID NO: 44. In some aspects, the 4-1BB costimulatory domain comprises a polynucleotide sequence about 95% identical to the polynucleotide sequence SEQ ID NO: 44. In some aspects, the 4-1BB costimulatory domain comprises a polynucleotide sequence about 98% identical to the polynucleotide sequence SEQ ID NO: 44. In some aspects, the 4-1BB costimulatory domain comprises a polynucleotide sequence about 99% identical to the polynucleotide sequence SEQ ID NO: 44.

In some aspects, the CD3zeta signaling domain comprises a polynucleotide sequence having about 85%, about 90%, about 91% about 92%, about 93%, about 94%, about 95%, about 96%, about 97%, about 98%, about 99%, or about 100% sequence identity to SEQ ID NO: 45. In some aspects, the CD3zeta signaling domain comprises a polynucleotide sequence having at least about 85%, at least about 90%, at least about 91%, at least about 92%, at least about 93%, at least about 94%, at least about 95%, at least about 96%, at least about 97%, at least about 98%, at least about 99%, or 100% sequence identity to SEQ ID NO: 45.

In some aspects, the CD3zeta signaling domain comprises a polynucleotide sequence at least 85% identical to the polynucleotide sequence of SEQ ID NO: 45. In some aspects, the CD3zeta signaling domain comprises a polynucleotide sequence at least 90% identical to the polynucleotide sequence of SEQ ID NO: 45. In some aspects, the CD3zeta signaling domain comprises a polynucleotide sequence at least 95% identical to the polynucleotide sequence of SEQ ID NO: 45. In some aspects, the CD3zeta signaling domain comprises a polynucleotide sequence at least 98% identical to the polynucleotide sequence of SEQ ID NO: 45. In some aspects, the CD3zeta signaling domain comprises a polynucleotide sequence at least 99% identical to the polynucleotide sequence of SEQ ID NO: 45. In some aspects, the CD3zeta signaling domain comprises a polynucleotide sequence corresponding to the polynucleotide sequence of SEQ ID NO: 45.

In some aspects, the CD3zeta signaling domain comprises a polynucleotide sequence about 85% identical to the polynucleotide sequence SEQ ID NO: 45. In some aspects, the CD3zeta signaling domain comprises a polynucleotide sequence about 90% identical to the polynucleotide sequence SEQ ID NO: 45. In some aspects, the CD3zeta signaling domain comprises a polynucleotide sequence about 95% identical to the polynucleotide sequence SEQ ID NO: 45. In some aspects, the CD3zeta signaling domain comprises a polynucleotide sequence about 98% identical to the polynucleotide sequence SEQ ID NO: 45. In some aspects, the CD3zeta signaling domain comprises a polynucleotide sequence about 99% identical to the polynucleotide sequence SEQ ID NO: 45.

A nucleic acid encoding a heavy chain variable domain or heavy chain and a nucleic acid encoding a light chain variable domain or light chain can be in the same polynucleotide or in different polynucleotides. A nucleic acid encoding a heavy chain variable domain or heavy chain and a nucleic acid encoding a light chain variable domain or light chain can be in the same vector or in different vectors.

Also provided herein are polynucleotides encoding an anti-CD94 antibody or antigen-binding fragment thereof disclosed herein or a domain thereof that are optimized, e.g., by codon/RNA optimization, replacement with heterologous signal sequences, and elimination of mRNA instability elements. Methods to generate optimized nucleic acids encoding an anti-CD94 antibody or antigen-binding fragment thereof or a domain thereof (e.g., heavy chain, light chain, VH domain, or VL domain) for recombinant expression by introducing codon changes (e.g., a codon change that encodes the same amino acid due to the degeneracy of the genetic code) and/or eliminating inhibitory regions in the mRNA can be carried out by adapting the optimization methods disclosed in, e.g., U.S. Pat. Nos. 5,965,726; 6,174,666; 6,291,664; 6,414,132; and 6,794,498, accordingly.

A polynucleotide encoding an antibody or antigen-binding fragment thereof disclosed herein or a domain thereof can be generated from nucleic acid from a suitable source (e.g., a hybridoma) using methods well known in the art (e.g., PCR and other molecular cloning methods). For example, PCR amplification using synthetic primers hybridizable to the 3′ and 5′ ends of a known sequence can be performed using genomic DNA obtained from hybridoma cells producing the antibody of interest. Such PCR amplification methods can be used to obtain nucleic acids comprising the sequence encoding the light chain and/or heavy chain of an antibody or antigen-binding fragment thereof. Such PCR amplification methods can be used to obtain nucleic acids comprising the sequence encoding the variable light chain region and/or the variable heavy chain region of an antibody or antigen-binding fragment thereof. The amplified nucleic acids can be cloned into vectors for expression in host cells and for further cloning, for example, to generate chimeric and humanized antibodies or antigen-binding fragments thereof.

Polynucleotides provided herein can be, e.g., in the form of RNA or in the form of DNA. DNA includes cDNA, genomic DNA, and synthetic DNA, and DNA can be double-stranded or single-stranded. If single stranded, DNA can be the coding strand or non-coding (anti-sense) strand. In some aspects, the polynucleotide is a cDNA or a DNA lacking one more endogenous introns. In some aspects, a polynucleotide is a non-naturally occurring polynucleotide. In some aspects, a polynucleotide is recombinantly produced. In some aspects, the polynucleotides are isolated. In some aspects, the polynucleotides are substantially pure. In some aspects, a polynucleotide is purified from natural components.

b. Cells and Vectors

In some aspects, provided herein are vectors (e.g., expression vectors) comprising polynucleotides comprising nucleotide sequences encoding anti-CD94 antibodies and antigen-binding fragments thereof or a domain thereof for recombinant expression in host cells, preferably in mammalian cells. Also provided herein are cells, e.g., host cells, comprising such vectors for recombinantly expressing anti-CD94 antibodies or antigen-binding fragments thereof disclosed herein. In a some aspects, provided herein are methods for producing an antibody or antigen-binding fragments thereof disclosed herein, comprising expressing such antibody or antigen-binding fragment thereof in a host cell.

In some aspects, recombinant expression of an antibody or antigen-binding fragment thereof or domain thereof disclosed herein (e.g., a heavy or light chain disclosed herein) that specifically binds to CD94 (e.g., human CD94) involves construction of an expression vector containing a polynucleotide that encodes the antibody or antigen-binding fragment thereof or domain thereof. Once a polynucleotide encoding an antibody or antigen-binding fragment thereof or domain thereof (e.g., heavy or light chain variable domain) disclosed herein has been obtained, the vector for the production of the antibody or antigen-binding fragment thereof can be produced by recombinant DNA technology using techniques well known in the art. Thus, methods for preparing a protein by expressing a polynucleotide containing an antibody or antigen-binding fragment thereof or domain thereof (e.g., light chain or heavy chain) encoding nucleotide sequence are disclosed herein. Methods which are well known to those skilled in the art can be used to construct expression vectors containing antibody or antigen-binding fragment thereof or domain thereof (e.g., light chain or heavy chain) coding sequences and appropriate transcriptional and translational control signals. These methods include, for example, in vitro recombinant DNA techniques, synthetic techniques, and in vivo genetic recombination. Also provided are replicable vectors comprising a nucleotide sequence encoding an antibody or antigen-binding fragment thereof disclosed herein, a heavy or light chain, a heavy or light chain variable domain, or a heavy or light chain CDR, operably linked to a promoter. Such vectors can, for example, include the nucleotide sequence encoding the constant region of the antibody or antigen-binding fragment thereof (see, e.g., International Publication Nos. WO 86/05807 and WO 89/01036; and U.S. Pat. No. 5,122,464), and variable domains of the antibody or antigen-binding fragment thereof can be cloned into such a vector for expression of the entire heavy, the entire light chain, or both the entire heavy and light chains.

An expression vector can be transferred to a cell (e.g., host cell) by conventional techniques, and the resulting cells can then be cultured by conventional techniques to produce an antibody or antigen-binding fragment thereof disclosed herein, e.g., an antibody or antigen-binding fragment thereof comprising any of the anti-CD94 antibodies disclosed herein.

In some aspects, disclosed herein is an isolated vector comprising any of the polynucleotides disclosed herein.

In some aspects, provided herein is a host cell comprising any of the polynucleotide disclosed herein.

In some aspects, the host cell is selected form the group consisting of CHO, HEK-293T, HeLa and BHK cells. In some aspects, the CHO cell is a CHO-K1SP cell.

In some aspects, disclosed herein is a method of producing an antibody or antigen-binding fragment thereof capable of binding to CD94, the method comprising (a) culturing any of the host cells disclosed herein in a cell culture under conditions which allow expression of the antibody or antigen-binding fragment thereof; and (b) recovering the antibody or antigen-binding fragment thereof from said cell culture. In some aspects, disclosed herein is an antibody or antigen-binding fragment thereof obtainable by any of the methods of production disclosed herein.

In some aspects, for the expression of double-chained antibodies or antigen-binding fragments thereof, vectors encoding both the heavy and light chains, individually, can be co-expressed in the host cell for expression of the entire immunoglobulin, as detailed below. In some aspects, a host cell contains a vector comprising a polynucleotide encoding both the heavy chain and light chain of an antibody disclosed herein or a domain thereof. In some aspects, a host cell contains two different vectors, a first vector comprising a polynucleotide encoding a heavy chain or a heavy chain variable region of an antibody or antigen-binding fragment thereof disclosed herein, and a second vector comprising a polynucleotide encoding a light chain or a light chain variable region of an antibody disclosed herein or a domain thereof. In some aspects, a first host cell comprises a first vector comprising a polynucleotide encoding a heavy chain or a heavy chain variable region of an antibody or antigen-binding fragment thereof disclosed herein, and a second host cell comprises a second vector comprising a polynucleotide encoding a light chain or a light chain variable region of an antibody or antigen-binding fragment thereof disclosed herein. In some aspects, a heavy chain/heavy chain variable region expressed by a first cell associated with a light chain/light chain variable region of a second cell to form an anti-CD94 antibody or antigen-binding fragment thereof disclosed herein. In some aspects, provided herein is a population of host cells comprising such first host cell and such second host cell.

In some aspects, provided herein is a population of vectors comprising a first vector comprising a polynucleotide encoding a light chain/light chain variable region of an anti-CD94 antibody or antigen-binding fragment thereof disclosed herein, and a second vector comprising a polynucleotide encoding a heavy chain/heavy chain variable region of an anti-CD94 antibody or antigen-binding fragment thereof disclosed herein. Alternatively, a single vector can be used which encodes, and is capable of expressing, both heavy and light chain polypeptides.

A variety of host-expression vector systems can be utilized to express antibodies and antigen-binding fragments thereof disclosed herein (see, e.g., U.S. Pat. No. 5,807,715). Such host-expression systems represent vehicles by which the coding sequences of interest can be produced and subsequently purified, but also represent cells which can, when transformed or transfected with the appropriate nucleotide coding sequences, express an antibody or antigen-binding fragment thereof disclosed herein in situ. These include but are not limited to microorganisms such as bacteria (e.g., E. coli and B. subtilis) transformed with recombinant bacteriophage DNA, plasmid DNA or cosmid DNA expression vectors containing antibody coding sequences; yeast (e.g., Saccharomyces Pichia) transformed with recombinant yeast expression vectors containing antibody coding sequences; insect cell systems infected with recombinant virus expression vectors (e.g., baculovirus) containing antibody coding sequences; plant cell systems (e.g., green algae such as Chlamydomonas reinhardtii) infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS (e.g., COS1 or COS), CHO, BHK, MDCK, HEK 293, NS0, PER.C6, VERO, CRL7O3O, HsS78Bst, HeLa, and NIH 3T3, HEK-293T, HepG2, SP210, R1.1, B-W, L-M, BSC1, BSC40, YB/20 and BMT10 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). In some aspects, cells for expressing antibodies and antigen-binding fragments thereof disclosed herein are CHO cells, for example CHO cells from the CHO GS System™ (Lonza). In some aspects, cells for expressing antibodies disclosed herein are human cells, e.g., human cell lines. In some aspects, a mammalian expression vector is pOptiVEC™ or pcDNA3.3. In some aspects, bacterial cells such as Escherichia coli, or eukaryotic cells (e.g., mammalian cells), especially for the expression of whole recombinant antibody molecule, are used for the expression of a recombinant antibody molecule. For example, mammalian cells such as Chinese hamster ovary (CHO) cells in conjunction with a vector such as the major intermediate early gene promoter element from human cytomegalovirus is an effective expression system for antibodies (Foecking M K & Hofstetter H (1986) Gene 45:101-105; and Cockett M I et al., (1990) Biotechnology 8:662-667). In some aspects, antibodies or antigen-binding fragments thereof disclosed herein are produced by CHO cells or NS0 cells.

In addition, a host cell strain can be chosen which modulates the expression of the inserted sequences, or modifies and processes the gene product in the specific fashion desired. Such modifications (e.g., glycosylation) and processing (e.g., cleavage) of protein products can contribute to the function of the protein. To this end, eukaryotic host cells that possess the cellular machinery for proper processing of the primary transcript, glycosylation, and phosphorylation of the gene product can be used. Such mammalian host cells include but are not limited to CHO, VERO, BHK, HeLa, MDCK, HEK 293, NIH 3T3, W138, BT483, Hs578T, HTB2, BT20 and T47D, NS0 (a murine myeloma cell line that does not endogenously produce any immunoglobulin chains), CRL7O3O, COS (e.g., COS1 or COS), PER.C6, VERO, HsS78Bst, HEK-293T, HepG2, SP210, R1.1, B-W, L-M, BSC1, BSC40, YB/20, BMT10 and HsS78Bst cells. In some aspects, anti-CD94 antibodies disclosed herein are produced in mammalian cells, such as CHO cells, e.g., CHO-K1 cells. In some aspects, anti-CD94 antibodies disclosed herein are produced in mammalian cells, such as HEK-293 cells.

In some aspects, a signal peptide is used in constructing a vector containing the VH and/or VL or the heavy and/or light chain of an antibody or antigen-binding fragment thereof provided herein.

Once an antibody or antigen-binding fragment thereof disclosed herein has been produced by recombinant expression, it can be purified by any method known in the art for purification of an immunoglobulin molecule, for example, by chromatography (e.g., ion exchange, affinity, particularly by affinity for the specific antigen after Protein A, and sizing column chromatography), centrifugation, differential solubility, or by any other standard technique for the purification of proteins. Further, the antibodies or antigen-binding fragments thereof disclosed herein can be fused to heterologous polypeptide sequences disclosed herein or otherwise known in the art to facilitate purification.

In some aspects, an antibody or antigen-binding fragment thereof disclosed herein is isolated or purified. Generally, an isolated antibody or antigen-binding fragment thereof is one that is substantially free of other antibodies or antigen-binding fragments thereof with different antigenic specificities than the isolated antibody or antigen-binding fragment thereof. For example, in some aspects, a preparation of an antibody or antigen-binding fragment thereof disclosed herein is substantially free of cellular material and/or chemical precursors.

In some aspects, the nucleic acids encoding the antibody or antigen-binding fragment thereof disclosed herein or the CAR disclosed herein are provided in a viral vector. In some aspects, the viral vector is a retrovirus, a lentivirus, or a foamy virus.

In some aspects, the viral vector containing the coding sequence for the antibody or antigen-binding fragment thereof disclosed herein or the CAR disclosed herein is a retroviral vector or a lentiviral vector. The term “retroviral vector” refers to a vector containing structural and functional genetic elements that are primarily derived from a retrovirus. The term “lentiviral vector” refers to a vector containing structural and functional genetic elements outside the LTRs that are primarily derived from a lentivirus.

The retroviral vectors for use herein can be derived from any known retrovirus (e.g., type c retroviruses, such as Moloney murine sarcoma virus (MoMSV), Harvey murine sarcoma virus (HaMuSV), murine mammary tumor virus (MuMTV), gibbon ape leukemia virus (GaLV), feline leukemia virus (FLV), spumavirus, Friend, Murine Stem Cell Virus (MSCV) and Rous Sarcoma Virus (RSV)). Retroviruses” of the disclosure also include human T cell leukemia viruses, HTLV-1 and HTLV-2, and the lentiviral family of retroviruses, such as Human Immunodeficiency Viruses, HIV-1, HIV-2, simian immunodeficiency virus (SIV), feline immunodeficiency virus (FIV), equine immunodeficiency virus (EIV), and other classes of retroviruses.

A lentiviral vector for use herein refers to a vector derived from a lentivirus, a group (or genus) of retroviruses that give rise to slowly developing disease. Viruses included within this group include HIV (human immunodeficiency virus; including HIV type 1, and HIV type 2); visna-maedi; a caprine arthritis-encephalitis virus; equine infectious anemia virus; feline immunodeficiency virus (FIV); bovine immune deficiency virus (BIV); and simian immunodeficiency virus (SIV). Preparation of the recombinant lentivirus can be achieved using the methods according to Dull et al. and Zufferey et al. (Dull et al., J. Virol., 1998; 72:8463-8471 and Zufferey et al., J. Virol. 1998; 72:9873-9880).

Retroviral vectors (i.e., both lentiviral and non-lentiviral) for use in the present disclosure can be formed using standard cloning techniques by combining the desired DNA sequences in the order and orientation disclosed herein (Current Protocols in Molecular Biology, Ausubel, F. M. et al. (eds.) Greene Publishing Associates, (1989), Sections 9.10-9.14 and other standard laboratory manuals; Eglitis, et al. (1985) Science 230:1395-1398; Danos and Mulligan (1988) Proc. Natl. Acad. Sci. USA 85:6460-6464; Wilson et al. (1988) Proc. Natl. Acad. Sci. USA 85:3014-3018; Armentano et al. (1990) Proc. Natl. Acad. Sci. USA 87:6141-6145; Huber et al. (1991) Proc. Natl. Acad. Sci. USA 88:8039-8043; Ferry et al. (1991) Proc. Natl. Acad. Sci. USA 88:8377-8381; Chowdhury et al. (1991) Science 254:1802-1805; van Beusechem et al. (1992) Proc. Natl. Acad. Sci. USA 89:7640-7644; Kay et al. (1992) Human Gene Therapy 3:641-647; Dai et al. (1992) Proc. Natl. Acad. Sci. USA 89:10892-10895; Hwu et al. (1993) J. Immunol 150:4104-4115; U.S. Pat. Nos. 4,868,116; 4,980,286; PCT Application WO 89/07136; PCT Application WO 89/02468; PCT Application WO 89/05345; and PCT Application WO 92/07573).

Suitable sources for obtaining retroviral (i.e., both lentiviral and non-lentiviral) sequences for use in forming the vectors include, for example, genomic RNA and cDNAs available from commercially available sources, including the Type Culture Collection (ATCC), Rockville, Md. The sequences also can be synthesized chemically.

### V. Pharmaceutical Compositions

Provided herein are compositions comprising an antibody or antigen-binding fragment thereof disclosed herein, a chimeric antigen receptor (CAR) disclosed herein, or an engineered T cell disclosed herein having the desired degree of purity in a physiologically acceptable carrier, excipient or stabilizer (Remington's Pharmaceutical Sciences (1990) Mack Publishing Co., Easton, PA). Acceptable carriers, excipients, or stabilizers are nontoxic to recipients at the dosages and concentrations employed.

In various aspects, compositions comprising an anti-CD94 antibody or antigen-binding fragment thereof, an anti-CD94 chimeric antigen receptor, or an engineered T cell targeting CD94 are provided in compositions with a pharmaceutically acceptable carrier (see, e.g., Gennaro, Remington: The Science and Practice of Pharmacy with Facts and Comparisons: Drugfacts Plus, 20th ed. (2003); Ansel et al., Pharmaceutical Dosage Forms and Drug Delivery Systems, 7th ed., Lippencott Williams and Wilkins (2004); Kibbe et al., Handbook of Pharmaceutical Excipients, 3rd ed., Pharmaceutical Press (2000)).

The pharmaceutical compositions disclosed herein are in one aspect for use as a medicament. The pharmaceutical compositions disclosed herein are in one aspect for use as a diagnostic, e.g., to detect the presence of CD94 in a sample obtained from a patient (e.g., a human patient).

The compositions to be used for in vivo administration can be sterile. This is readily accomplished by filtration through, e.g., sterile filtration membranes.

In some aspects, pharmaceutical compositions are provided, wherein the pharmaceutical composition comprises anti-CD94 antibodies or antigen-binding fragments thereof disclosed herein, a chimeric antigen receptor disclosed herein, or an engineered T cell disclosed herein and a pharmaceutically acceptable carrier.

In some aspects, disclosed herein is a pharmaceutical composition comprising any of the antibody or antigen-binding fragments thereof disclosed herein.

In some aspects, disclosed herein is a pharmaceutical composition comprising any of the chimeric antigen receptors disclosed herein.

In some aspects, disclosed herein is a pharmaceutical composition comprising any of the engineered T cells disclosed herein. In some aspects, the engineered T cells are CAR-T cells.

In some aspects, disclosed herein is a pharmaceutical composition comprising any of the vectors disclosed herein.

In some aspects, the pharmaceutical composition further comprises a pharmaceutically acceptable excipient.

### VI. Methods of Using

In some aspects, provided herein is a method of treating cancer in an individual, the method comprising administering to the individual a therapeutically effective amount of any of the anti-CD94 antibodies or antigen-binding portions thereof, chimeric antigen receptors (CARs), engineered T cells, or compositions comprising the same as provided herein.

In some aspects, provided herein is a method of treating cancer in an individual, the method comprising administering to the individual a therapeutically effective amount of any of the antibody or antigen-binding fragments thereof disclosed herein.

In some aspects, provided herein is a method of treating cancer in an individual, the method comprising administering to the individual a therapeutically effective amount of any of the engineered T cells disclosed herein. In some aspects, the method comprises administering to the individual a therapeutically effective amount of CAR Natural Killer cells, CAR macrophages, or CAR Natural Killer T Cells. In some aspects, the method comprises administering to the individual a therapeutically effective amount of an engineered immune cell. In some aspects, the engineered immune cell comprises any of the chimeric antigen receptors disclosed herein. In some aspects, the engineered immune cell is a macrophage. In some aspects, the engineered immune cell is a monocyte.

In some aspects, provided herein is a method of treating cancer in an individual, the method comprising administering to the individual a therapeutically effective amount of any of the vectors disclosed herein.

In some aspects, provided herein is a method of treating cancer in an individual, the method comprising administering to the individual a therapeutically effective amount of any of the pharmaceutical compositions provided herein.

In some aspects, the cancer is a leukemia.

In some aspects, the leukemia is T cell leukemia, T cell large granular leukemia, Natural Killer cell large granular leukemia, or Natural Killer cell leukemia.

In some aspects, the cancer is a lymphoma.

In some aspects, the lymphoma is T cell lymphoma, extranodal Natural Killer/T cell lymphoma, hepatosplenic T cell lymphoma, angioimmunoblastic T cell, or anaplastic large cell lymphoma.

In some aspects, the cancer is a CD94 expressing cancer.

In some aspects, the cancer is lung cancer, bladder cancer, or melanoma.

In some aspects, provided herein is a method of treating or preventing graft-rejection of a transplant in a patient, the method comprising administering to the individual a therapeutically effective amount of any of the antibody or antigen-binding fragments thereof disclosed herein.

In some aspects, provided herein is a method of treating or preventing graft-rejection of a transplant in a patient, the method comprising administering to the individual a therapeutically effective amount of any of the chimeric antigen receptors provided herein.

In some aspects, provided herein is a method of treating or preventing graft-rejection of a transplant in a patient, the method comprising administering to the individual a therapeutically effective amount of any of the engineered T cells disclosed herein.

In some aspects, provided herein is a method of treating or preventing graft-rejection of a transplant in a patient, the method comprising administering to the individual a therapeutically effective amount of any of the vectors disclosed herein.

In some aspects, provided herein is a method of treating or preventing graft-rejection of a transplant in a patient, the method comprising administering to the individual a therapeutically effective amount of any of the pharmaceutical compositions provided herein.

In some aspects, the transplant is an allogenic transplant.

In some aspects, the transplant is an organ transplant.

In some aspects, the transplant is a hematopoietic cell transplant.

In some aspects, the transplant is an induced pluripotent cell therapy.

In some aspects, provided herein is a method of modulating an immune response in a subject, the method comprising administering to the individual therapeutically effective amount of any of the antibody or antigen-binding fragments thereof disclosed herein.

In some aspects, provided herein is a method of modulating an immune response in a subject, the method comprising administering to the individual therapeutically effective amount of any of the chimeric antigen receptors provided herein.

In some aspects, provided herein is a method of modulating an immune response in a subject, the method comprising administering to the individual therapeutically effective amount of any of the engineered T cells disclosed herein.

In some aspects, provided herein is a method of modulating an immune response in a subject, the method comprising administering to the individual therapeutically effective amount of any of the vectors disclosed herein.

In some aspects, provided herein is a method of modulating an immune response in a subject, the method comprising administering to the individual therapeutically effective amount of any of the pharmaceutical compositions provided herein.

In some aspects, the immune response is enhanced.

In some aspects, the immune response is mediated by Natural Killer cells and/or T cells. In some aspects, the antibody or antigen-binding fragment thereof or composition disclosed herein can be delivered to a subject by a variety of routes, such as parenteral, subcutaneous, intravenous, intradermal, transdermal, intrathecal, and intranasal. In some aspects, the antibody or antigen-binding fragment thereof or composition is administered by an intraperitoneal route.

The amount of an antibody or antigen-binding fragment thereof or composition which will be effective in the treatment or prevention of a condition will depend on the nature of the disease. The precise dose to be employed in a composition will also depend on the route of administration, and the seriousness of the disease.

In some aspects, described herein is a method of modulating an immune response in a subject, the method comprising administering to the individual therapeutically effective amount of any of the antibody or antigen-binding fragments thereof described herein, any of the chimeric antigen receptors described herein, any of the engineered T cells disclosed herein, or any of the pharmaceutical compositions disclosed herein.

In some aspects, the immune response is enhanced.

In some aspects, the immune response is mediated by Natural Killer cells and/or T cells. In some aspects, the Natural Killer cells and/or T cells mediate the immune response of an autoimmune disease. In some aspects, the autoimmune disease is a Systemic Autoimmune Disease. In some aspects, the systemic autoimmune disease is systemic lupus erythematosus (SLE), Sjögren's Syndrome, Systemic Sclerosis, Rheumatoid Arthritis (RA), Multiple Sclerosis, type 1 diabetes mellitus (T1DM), or autoimmune liver disease (ALD).

An anti-CD94 antibody or antigen-binding fragment thereof disclosed herein can be used to assay CD94 protein levels or CD94+ cells in a biological sample using classical methods known to those of skill in the art, including immunoassays, such as the enzyme linked immunosorbent assay (ELISA), fluorescence-activated cell sorting (FACS), immunohistochemistry (IHC), immunoprecipitation, and Western blotting. Suitable antibody assay labels are known in the art and include enzyme labels, such as, glucose oxidase; radioisotopes, such as iodine (¹²⁵I, ¹²¹I), carbon (¹⁴C), sulfur (³⁵S), tritium (³H), indium (¹²¹In), and technetium (⁹⁹Tc); luminescent labels, such as luminol; and fluorescent labels, such as fluorescein and rhodamine, and biotin. Such labels can be used to label an antibody or antigen-binding fragment thereof disclosed herein. Alternatively, a second antibody or antigen-binding fragment thereof that recognizes an anti-CD94 antibody or antigen-binding fragment thereof disclosed herein can be labeled and used in combination with an anti-CD94 antibody or antigen-binding fragment thereof to detect CD94 protein levels (e.g., soluble CD94 protein levels).

As used herein, the term “biological sample” refers to any biological sample obtained from a subject, cell line, tissue, or other source of cells potentially expressing CD94. Methods for obtaining tissue biopsies and body fluids from animals (e.g., humans) are well known in the art. A biological sample can also be a blood sample.

Anti-CD94 antibodies and antigen-binding fragments thereof disclosed herein can carry a detectable or functional label.

Examples of detectable moieties that can be used herein include but are not limited to radioactive isotopes, phosphorescent chemicals, chemiluminescent chemicals, fluorescent chemicals, enzymes, fluorescent polypeptides, and epitope tags. The detectable moiety can be a member of a binding pair, which is identifiable via its interaction with an additional member of the binding pair, and a label which is directly visualized.

When fluorescence labels are used, currently available microscopy and fluorescence-activated cell sorter analysis (FACS) or combination of both methods procedures known in the art can be utilized to identify and to quantitate the specific binding members. Anti-CD94 antibodies or antigen-binding fragments thereof disclosed herein can carry a fluorescence label. Exemplary fluorescence labels include, for example, reactive and conjugated probes, e.g., Aminocoumarin, Fluorescein and Texas red, Alexa Fluor dyes, Cy dyes and DyLight dyes. An anti-CD94 antibody can carry a radioactive label, such as the isotopes ³H, ¹⁴C, ³²P, ³⁵S, ³⁶Cl, ⁵¹Cr, ⁵⁷Co, ⁵⁸Co, ⁵⁹Fe, ⁶⁷Cu, ⁹⁰Y, ⁹⁹Tc, ¹¹¹In, ¹¹⁷Lu, ¹²¹I, ¹²⁴I, ¹³¹I, ¹⁹⁸Au, ²¹¹At, ²¹³Bi, ²²⁵Ac and ¹⁸⁶Re. When radioactive labels are used, currently available counting procedures known in the art can be utilized to identify and quantitate the specific binding of anti-CD94 antibody or antigen-binding fragment to CD94 (e.g., human CD94). In the instance where the label is an enzyme, detection can be accomplished by any of the presently utilized colorimetric, spectrophotometric, fluorospectrophotometric, amperometric, or gasometric techniques as known in the art. This can be achieved by contacting a sample or a control sample with an anti-CD94 antibody or antigen-binding fragment thereof under conditions that allow for the formation of a complex between the antibody or antigen-binding fragment thereof and CD94. Any complexes formed between the antibody or antigen-binding fragment thereof and CD94 are detected and compared in the sample and the control.

In some aspects, methods for in vitro detecting CD94 in a sample, comprising contacting said sample with an antibody or antigen-binding fragment thereof, are provided herein, and optionally detecting binding of the antibody or antigen-binding fragment thereof to CD94. In some aspects, provided herein is the use of an antibody or antigen-binding fragment thereof provided herein, for in vitro detecting CD94 in a sample. In one aspect, provided herein is an antibody or antigen-binding fragment thereof or pharmaceutical composition provided herein for use in the detection of CD94 (e.g., soluble CD94) in a subject or a sample obtained from a subject.

In some aspects, the subject is a mammal. In some aspects, the mammal is selected from the group consisting of a human, a mouse, a hamster, a rabbit, a nonhuman primate, a guinea pig, a rat, a zebrafish, a pig, a sheep, a cat, or a dog. In some aspects, the subject is a human.

### VII. Kits

Provided herein are kits comprising one or more antibodies or antigen-binding fragments thereof disclosed herein or conjugates (e.g., detection conjugates) thereof.

In some aspects, the kit comprises one or more of the chimeric antigen receptors provided herein.

In some aspects, the kit comprises one or more of any of the engineered T cells disclosed herein.

In some aspects, the kit comprises one or more of any of the vectors disclosed herein.

In some aspects, the kit comprises one or more of any of the pharmaceutical compositions provided herein.

In some aspects, the kit further comprises instructions for use.

In some aspects, the kit is used for treating a subject in need thereof.

In some aspects, the kits will thus include, in suitable container means, cells, proteins or nucleic acid constructs or related reagents of the present invention. In some aspects, means of taking a sample from an individual and/or of assaying the sample may be provided in the kit. In some aspects, the kit includes cells, buffers, cell media, vectors, primers, restriction enzymes, salts, and so forth, for example.

The components of the kits may be packaged either in aqueous media or in lyophilized form. The container means of the kits will generally include at least one vial, test tube, flask, bottle, syringe or other container means, into which a component may be placed, and preferably, suitably aliquoted. Where there is more than one component in the kit, the kit also will generally contain a second, third or other additional container into which the additional components may be separately placed. However, various combinations of components may be comprised in a vial. Such containers may include injection or blow-molded plastic containers into which the desired vials are retained.

When the components of the kit are provided in one and/or more liquid solutions, the liquid solution is an aqueous solution, with a sterile aqueous solution being particularly preferred. The compositions may also be formulated into a syringeable composition. In which case, the container means may itself be a syringe, pipette, and/or other such like apparatus, from which the formulation may be applied to an infected area of the body, injected into an animal, and/or even applied to and/or mixed with the other components of the kit. However, the components of the kit may be provided as dried powder(s). When reagents and/or components are provided as a dry powder, the powder can be reconstituted by the addition of a suitable solvent. It is envisioned that the solvent may also be provided in another container means.

### EXAMPLES

### Example 1. Analysis of Anti-CD94 Monoclonal Antibody Specificity

Anti-CD94 monoclonal antibodies were designed to for treating NK cell or T cell lymphoproliferative disorders.

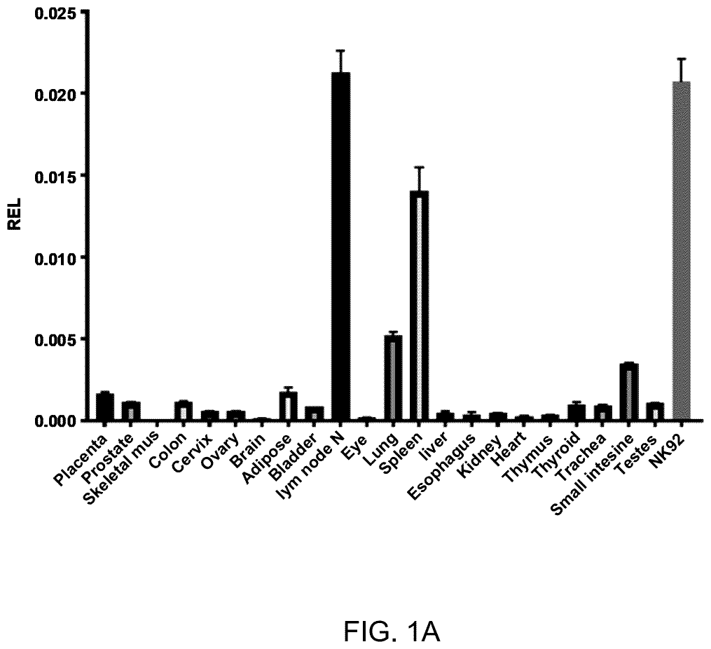

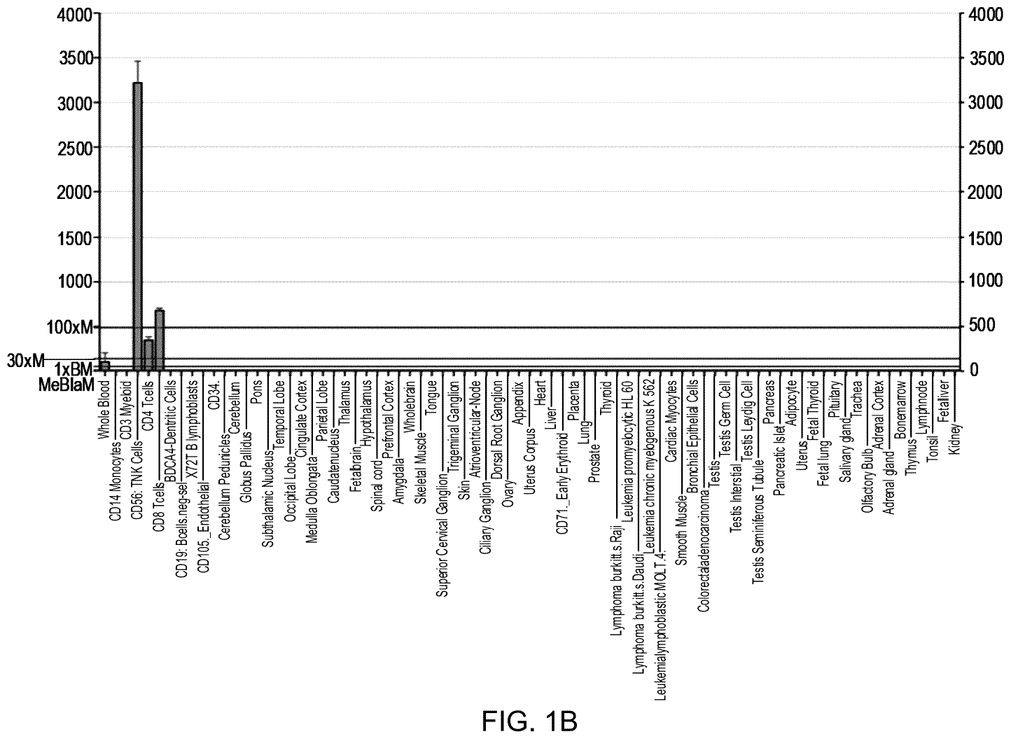

First, CD94 transcript expression was assessed in normal tissues. Human Total RNA Survey Panel containing RNA from 21 normal human tissues was obtained from Applied Biosystems, Foster City, CA. About 10 μg of total RNA from each source was reverse transcribed into cDNA with Superscript III kit (Invitrogen). Quantitative PCR was performed with CD94 and β-actin primers using the following conditions: 50° C. for 2 minutes, 94° C. for 10 minutes, followed by 94° C. for 15 seconds, and 60° C. for 60 seconds for 40 cycles on Applied Biosystems StepOne™/StepOnePlus™ Real-Time PCR System. The expression of CD94 mRNA relative to the β-actin mRNA was calculated in each sample (FIG. 1A).

High levels of CD94 mRNA were detected in lymphoid organs, lymph node, spleen, and NK cells (FIG. 1A). Lower levels of CD94 mRNA were detected in lung and small intestine, which are known to contain small amounts of lymphoid tissue.

A publicly available gene expression profiling database, BioGPS (http://biogps.org), was used to analyze CD94 mRNA expression in 79 normal human tissues and lymphoid tissue subtypes by high-density oligonucleotide arrays. The analysis determined that CD94 transcript was highly expressed in peripheral blood NK cells, while low levels were observed in CD8 T cells. CD94 mRNA was not expressed in other normal tissues (FIG. 1B).

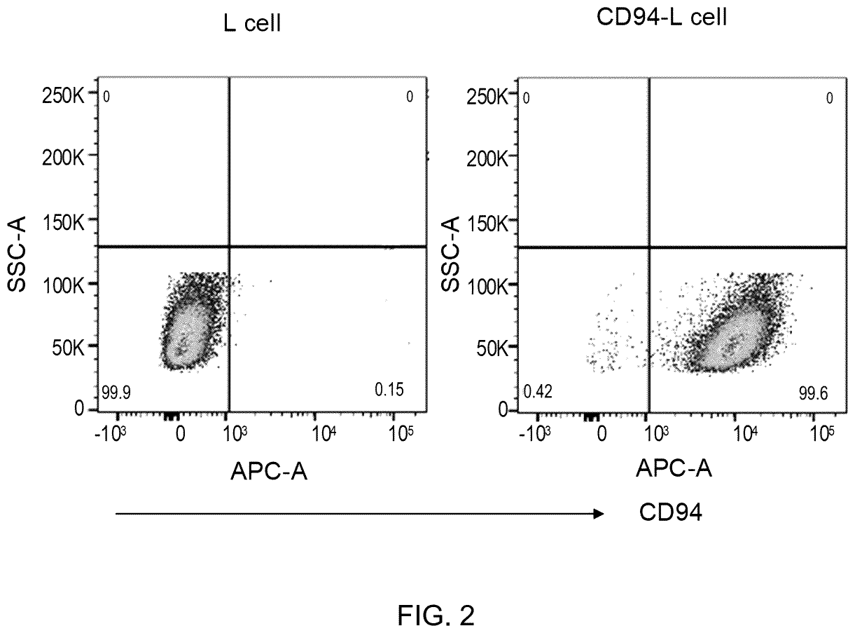

Next, an anti-human CD94 monoclonal antibody was generated (“UT-CD94”). Human CD94 was stably transfected into L cells (a mouse fibroblast cell line) and the CD94-expressing L cells were used to immunize BALB/c mice. Splenocytes from immunized mice were isolated and hybridized with mouse myeloma cells to derive a monoclonal antibody targeting human CD94 by hybridoma technology (FIG. 2).

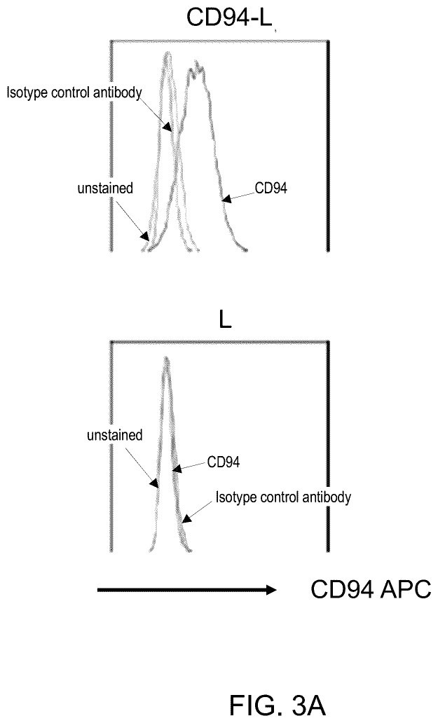

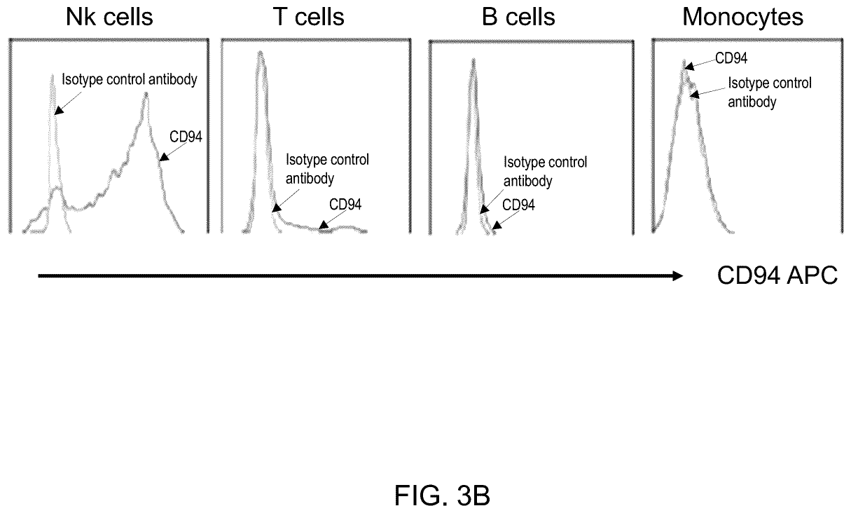

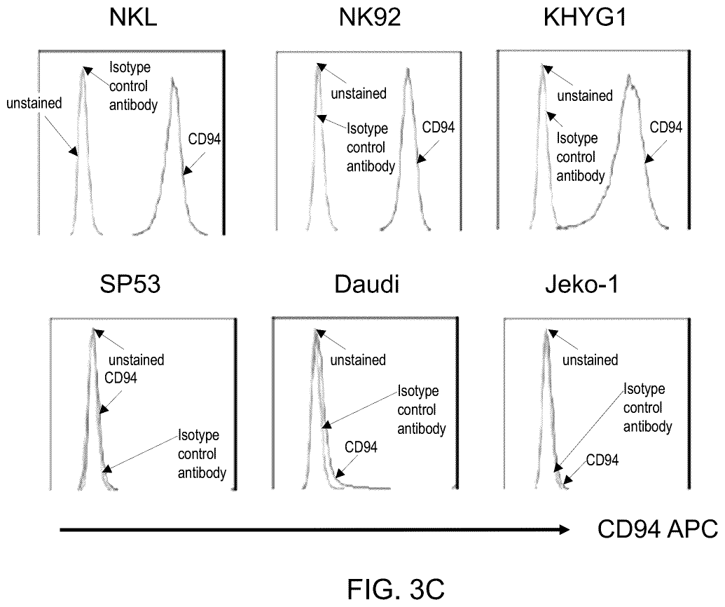

Specificity of the UT-CD94 monoclonal antibody was then tested by flow cytometry. The UT-CD94 monoclonal antibody was fluorochrome conjugated with AF647 and used to stain NK-cell lymphoma and leukemia cell lines as well as normal donor peripheral blood mononuclear cells (PBMC). Antibodies against CD3, CD20, CD14, and CD56 were also used to stain the cells. The UT-CD94 monoclonal antibody specifically stained human CD94-expressing L cells and human peripheral blood NK cells (FIG. 3A), but did not stain parental L cells, human B cells, T cells, or monocytes (FIG. 3B). The UT-CD94 monoclonal antibody also bound to NK lymphoma and leukemia cell lines (NK-92, NKL, KHYG-1), but did not bind to the B cell lymphoma cell lines (SP53, Daudi, and Jeko-1) (FIG. 3C). The flow cytometry analysis demonstrated that the UT-CD94 monoclonal antibody had specificity for CD94 expressing cells.

### Example 2. Analysis of CD94-Specific CAR-T Cell Cytotoxicity

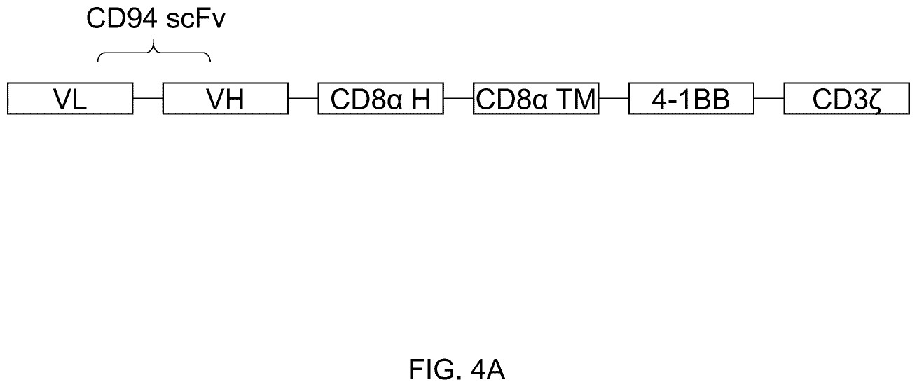

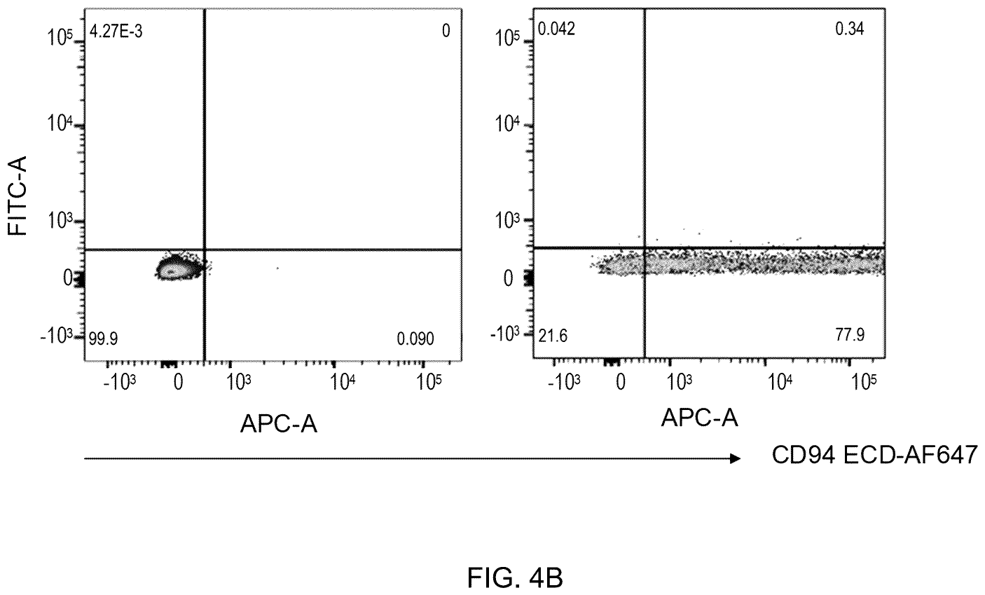

The VH and VL sequence of the UT-CD94 monoclonal antibody of Example 1 were used to generate an anti-CD94 chimeric antigen receptor (CAR). The VH and VL sequence of the UT-CD94 monoclonal antibody were converted to a single chain variable fragment (scFv) and an anti-CD94 CAR (“UT-CD94-CAR”) was generated using a CD8α hinge, a CD8α transmembrane domain, a 4-1BB signaling domain, and a CD3zeta signaling domain (FIG. 4A). The entire CAR sequence was incorporated into a lentivirus vector (pLVMG). The CDRs of the CAR amino acid and nucleotide sequences are shown above in Tables 1-3 (amino acid sequences) and Tables 4-6 (nucleotide sequences).

Normal donor T cells were purified from buffy coat, activated for 2-3 days with anti-CD3, anti-CD28, and anti-CD2 antibodies in the presence of IL-2, and then transduced with a lentivirus containing the UT-CD94-CAR. The transduction efficiency was assessed by staining with His-tagged CD94 extra cellular domain protein (ACROBiosystems, USA) followed by flow cytometry analysis with the anti-His AF647-conjugated antibody. The flow cytometry analysis for surface expression of the UT-CD94-CAR using CD94 extracellular domain protein staining revealed high transduction efficiency after 72 hours (FIG. 4B).

Next, the cytotoxicity of UT-CD94-CAR T cells were analyzed against NK-92 (NK-cell lymphoma cell line) and NKL (NK-cell leukemia cell line). NK-92 and NKL were labeled with CellTrace™ Far Red cell staining kit (Thermofisher), while the B cell lymphoma cell line SP53 (mantle cell lymphoma cell line) was stained with CFSE (Thermofisher). The NK-92, NKL, and SP53 cell lines were then co-cultured with UT-CD94-CAR-transduced T cells at 1:1:1 Effector:Tumor 1:Tumor 2 (E:T1:T2) ratio. The percentage of dead cells were determined by flow cytometry at days 1, 2, 3 and 4 after staining for dead cells (Thermofisher). The cytotoxicity of UT-CD94-CAR T cells was also analyzed against L cells (a mouse fibroblast cell line) and the human CD94-expressing L cells. Untransduced T cells or tumor alone were used as negative controls.

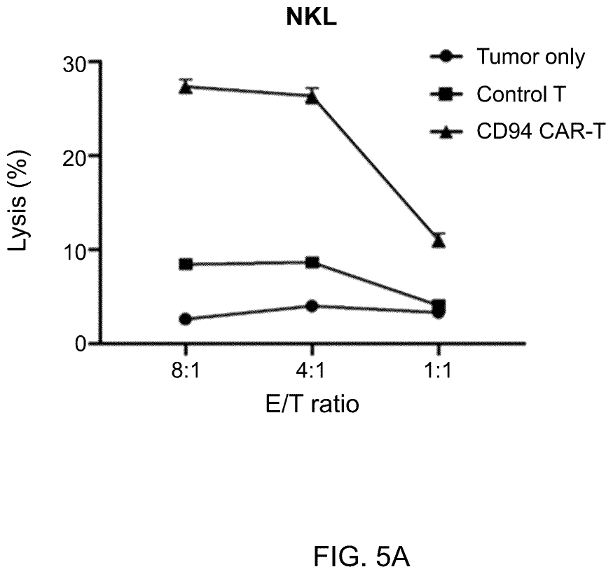

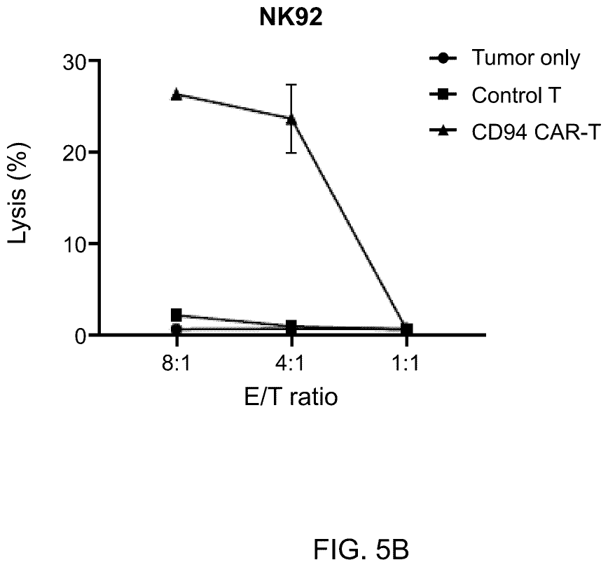

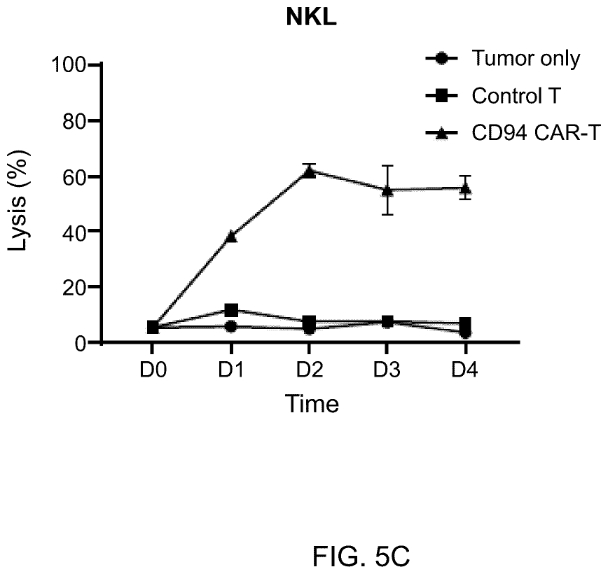

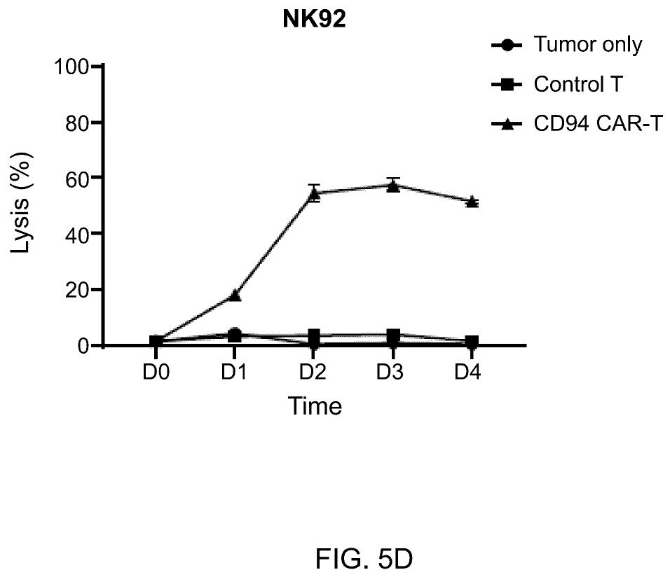

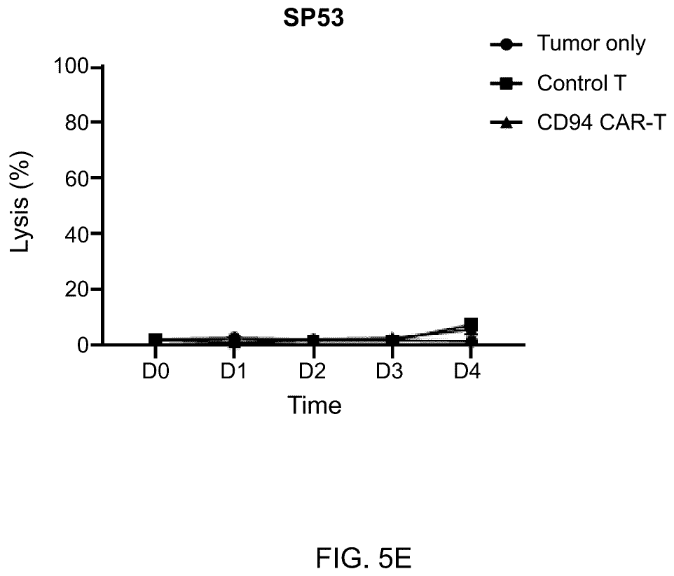

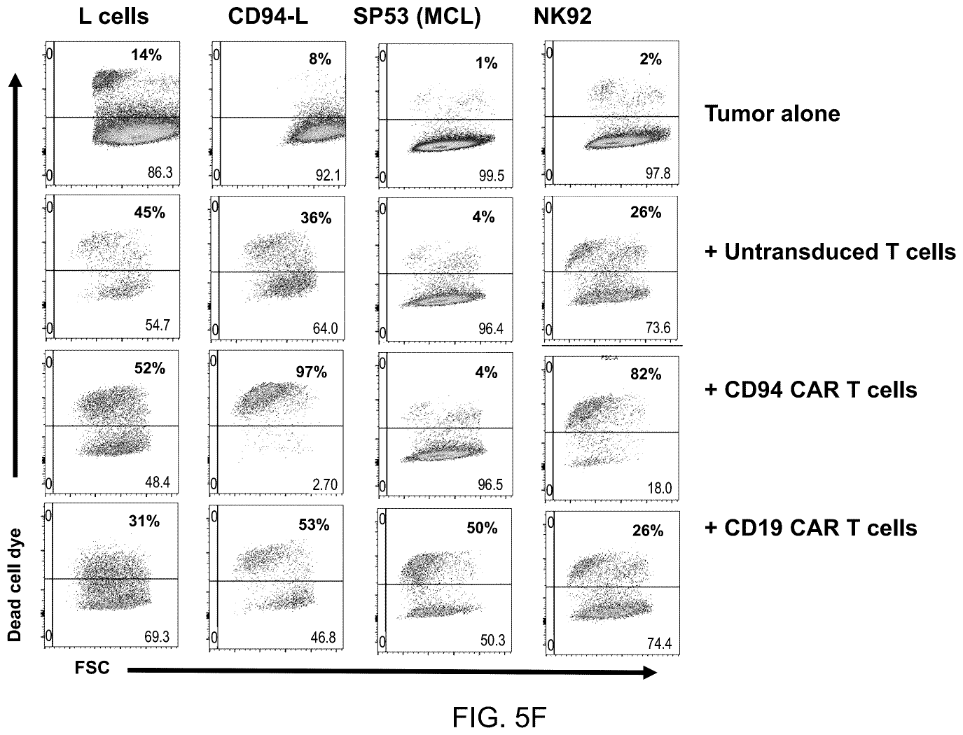

UT-CD94-CAR T cells induced significant lysis of CD94-positive NK lymphoma and leukemia cell lines (NK-92 and NKL) and L cells transfected with human CD94 but not CD94-negative B-cell lymphoma cell line (SP-53) or parental L cells (FIG. 5A-5F). There was no significant lysis observed with untransduced T cells.

This example demonstrates that the UT-CD94-CAR T cell disclosed herein shows high specificity and high cytotoxicity activity towards CD94 expressing cells.

### Example 3. Chimeric Anti-CD94 Antibody Blocks HLA-E Interactions

A chimeric anti-human CD94 antibody (“cUT-CD94”) was synthesized by replacing the mouse Fc with human IgG1 Fc in the murine UT-CD94 monoclonal antibody that was generated in example 1 using hybridoma technology. The murine and chimeric anti-human CD94 antibodies were then used in various in vitro assays.

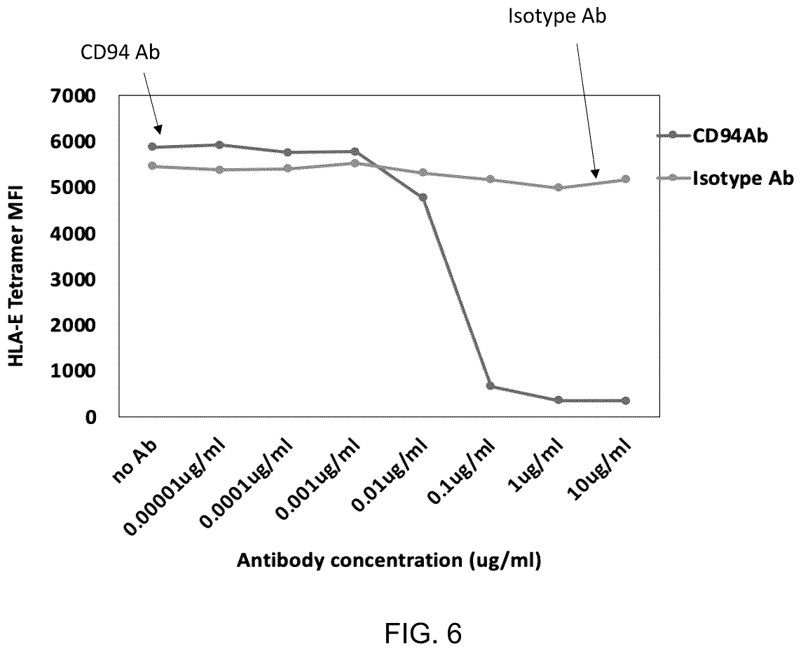

Since CD94 can function as a stimulatory or inhibitory receptor when it heterodimerizes with NKG2C or NKG2A, respectively, it was determined whether the cUT-CD94 antibody can block interaction with its ligand, HLA-E. The NK leukemia cell line, KHYG-1, was incubated with different concentrations of the cUT-CD94 antibody or isotype control antibody for 1 hour and then the cells were stained with HLA-E tetramer and analyzed by flow cytometry. The results showed that the cUT-CD94 antibody blocked interaction with HLA-E (FIG. 6).

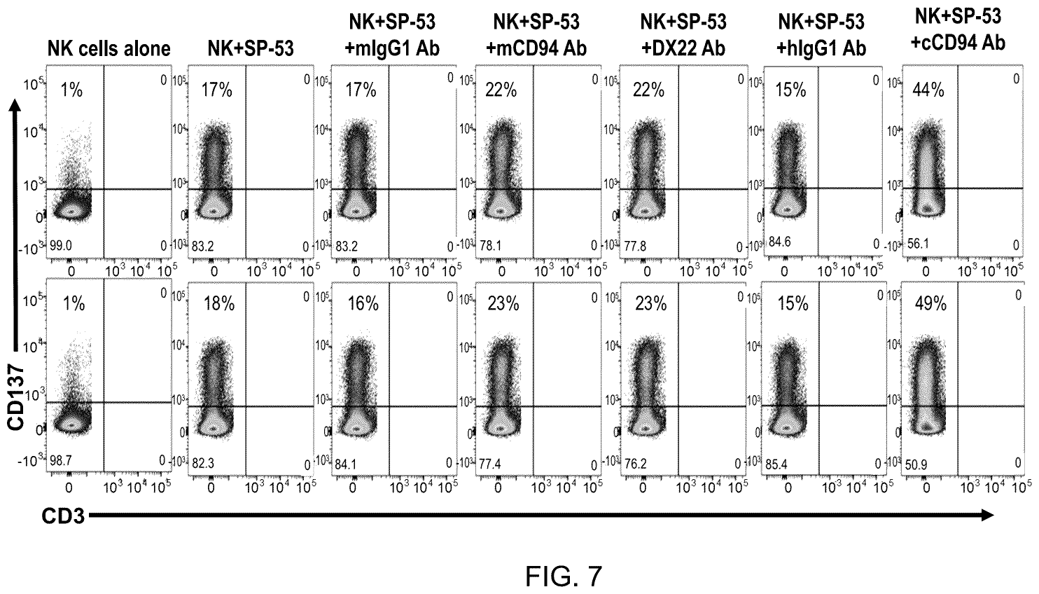

Since interaction of NKG2A/CD94 heterodimer with its ligand, HLA-E can lead to inhibition of the function of NK cells; it was determined whether blocking CD94 could lead to enhanced activation of NK cells. NK cells were isolated from normal donor peripheral blood mononuclear cells by magnetic cell separation and incubated with HLA-E-expressing SP-53 mantle cell lymphoma cell line at Effector:Target (E:T) ratio of 2:1 in duplicate wells in the presence or absence of the UT-CD94 antibody (mCD94 Ab) and CUT-CD94 antibody (cCD94 Ab) or commercial murine anti-CD94 antibody (clone DX22 from Biolegend Catalog #305502) and their respective isotype controls (mouse IgG1 isotype control antibody from Biolegend Catalog #401402 and human IgG1 isotype control antibody from Biocell Catalog #BE0297). All antibodies were used at a concentration of 10 μg/mL. CD137 expression was assessed by flow cytometry after overnight incubation (FIG. 7).

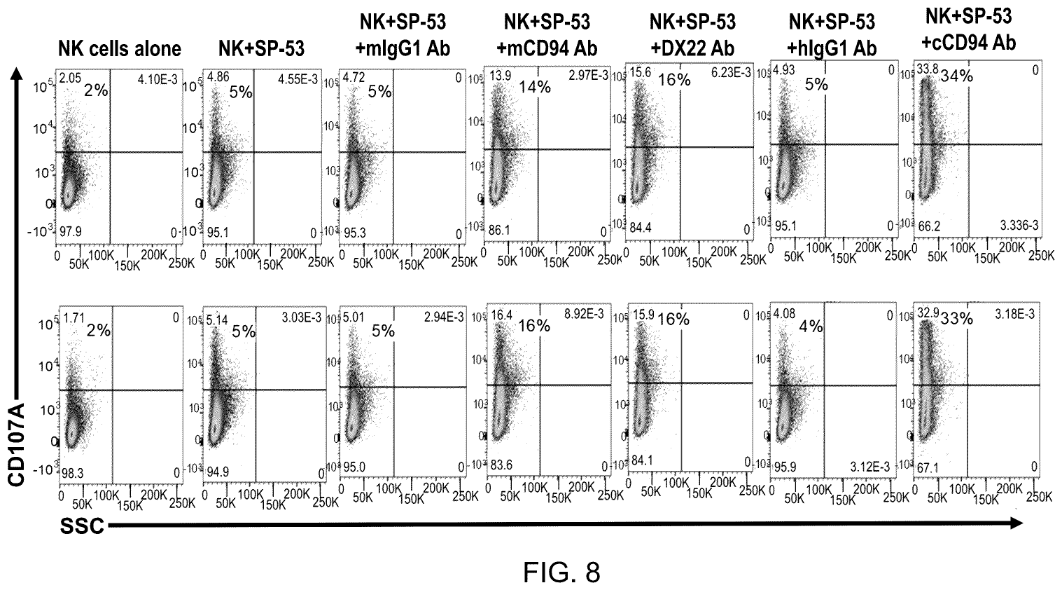

For CD107a expression analysis, NK cells were isolated from normal donor peripheral blood mononuclear cells by magnetic cell separation, activated with IL-2 (1200 IU/mL) overnight, and incubated with HLA-E-expressing SP-53 mantle cell lymphoma cell line at Effector:Target (E:T) ratio of 2:1 in duplicate wells in the presence or absence of the UT-CD94 antibody (mCD94 Ab) and cUT-CD94 antibody (cCD94 Ab) or commercial murine anti-CD94 antibody (clone DX22 from Biolegend Catalog #305502) and their respective isotype controls (mouse IgG1 isotype control antibody from Biolegend Catalog #401402 and human IgG1 isotype control antibody from Biocell Catalog #BE0297). All antibodies were used at a concentration of 10 μg/mL. CD107a expression was assessed by flow cytometry after 16 hours of incubation (FIG. 8).

It was observed that blocking CD94 with both UT-CD94 and CUT-CD94 antibodies resulted in increased activation of NK cells as determined by CD137 expression (FIG. 7) and CD 107a degranulation (FIG. 8). This increased expression of CD137 and CD107a was significantly higher with cUT-CD94 antibody compared to a commercial murine anti-CD94 antibody (clone DX22 from Biolegend). Specifically, CD137 expression was found in 44-49% of cells treated with the cUT-CD94 antibody, which was significantly higher than the 22-23% of cells with CD137 expression after treatment with either the UT-CD94 antibody (mCD94 Ab) or the commercial murine anti-CD94 antibody (DX22 Ab). Furthermore, CD107a expression was found in 33-34% of cells treated with the cUT-CD94 antibody, which was significantly higher than the 16% of cells with CD107a expression after treatment with the commercial murine anti-CD94 antibody (DX22) and the 14-16% of cells with CD107a expression after treatment with the UT-CD94 antibody generated in example 1 (mCD94 Ab). Taken together, these results show that the cUT-CD94 antibody generated herein is more effective at driving CD107a and CD137 expression than current commercial embodiments as demonstrated by the DX22 murine anti-CD94 antibody.

### Example 4. Anti-CD94 Antibody Drug Conjugate (Prophetic)

Using the UT-CD94 antibody of Example 1 or the chimeric cUT-CD94 antibody of Example 3, an anti-CD94 antibody drug conjugate (AdC) is generated. The anti-CD94 antibody is conjugated to a cytotoxic compound via a linker known in the art, such as those disclosed in Kostoba et al., Pharmaceuticals, 2021; 14(5):442; Tong et al., Molecules, 2021 Sep. 27; 26(19):5847; and Drago et al., Nat Rev Clin Oncol, 2021 June; 18(6):327-344. doi: 10.1038/s41571-021-00470-8. Epub 2021 Feb. 8. The cytotoxicity of the anti-CD94 AdC is analyzed against NK-cell lymphoma or leukemia lines NK-92 and NKL. NK-92 and NKL is labeled with CellTrace™ Far Red cell staining kit (Thermofisher), and the B-cell lymphoma cell line SP53 (mantle cell lymphoma cell line) is stained with CFSE (Thermofisher). The NK-92, NKL and SP53 cell lines are co-cultured with the anti-CD94 AdC. The percentage of dead cells is determined by flow cytometry at days 1, 2, 3 and 4 after staining for dead cells (Thermofisher). Tumor cells alone are used as negative controls.

### Example 5. CD94 Expression in Normal and Tumor Tissues

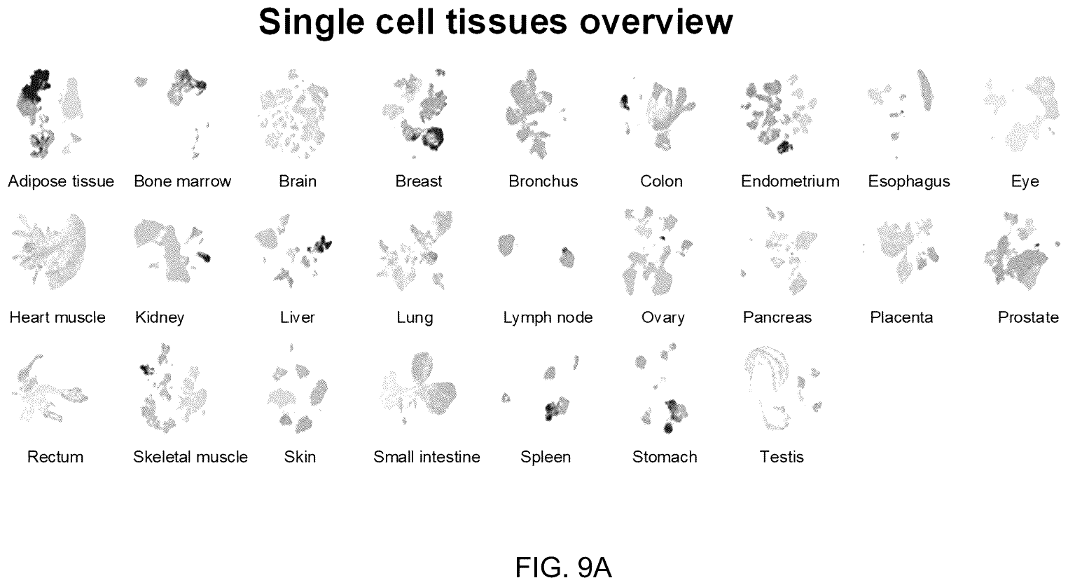

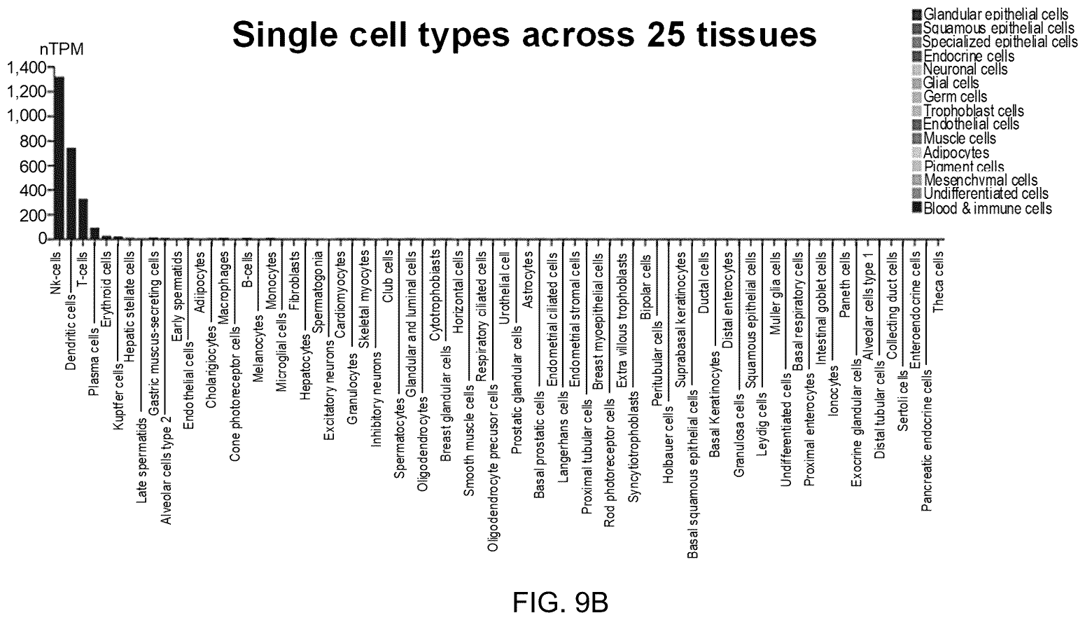

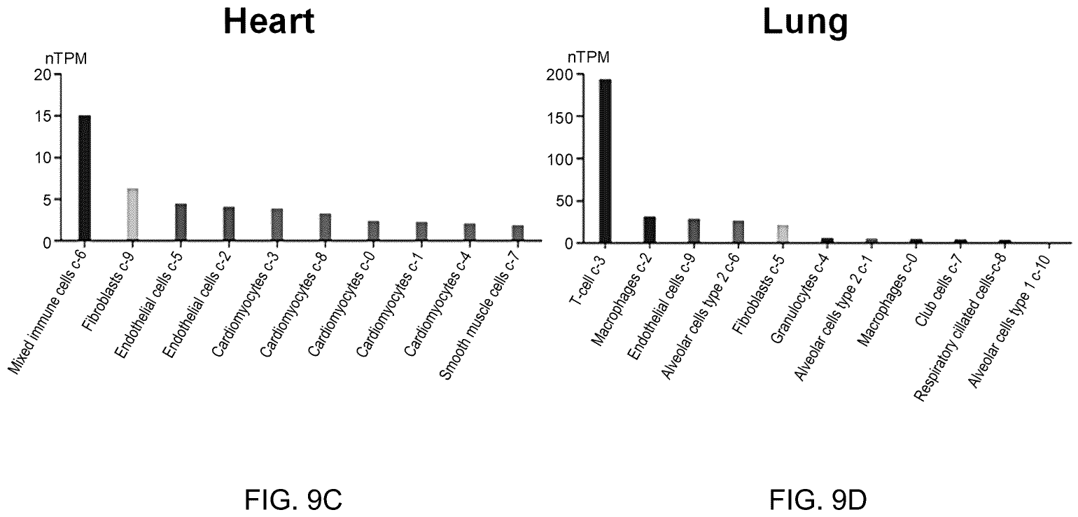

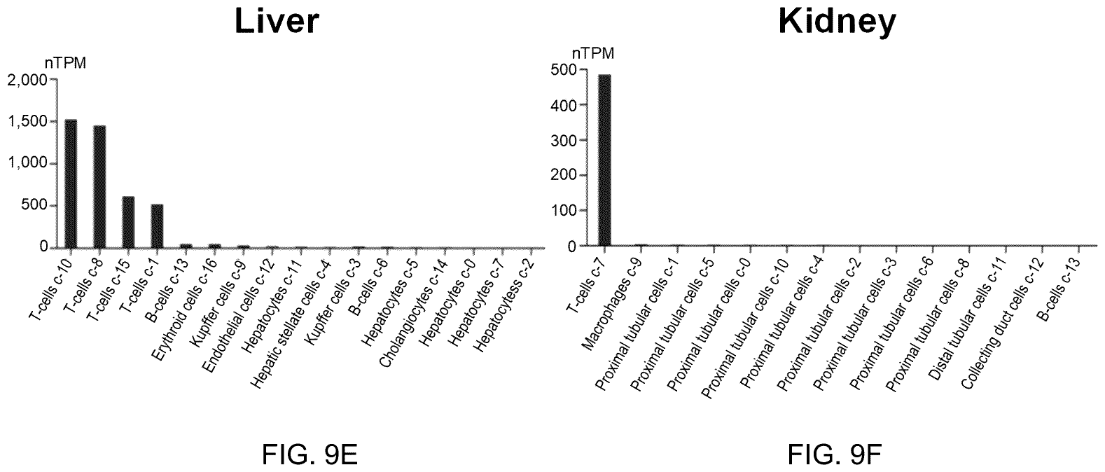

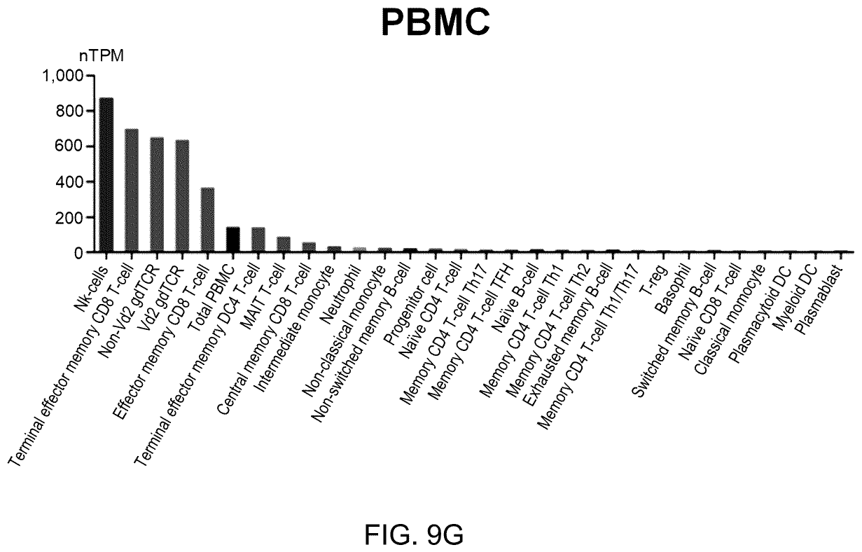

CD94 transcript expression is restricted in normal tissues. Analysis of single cell RNAseq (scRNASeq) data from the Human Protein Atlas project revealed that CD94 mRNA transcript was only present in infiltrating immune cells with highest expression in NK cells across 25 normal tissues including vital organs such as heart, lung, liver, and kidney. Importantly, CD94 transcripts were not detected in epithelial cells, endothelial cells, muscle, adipocytes, cardiomyocytes, hepatocytes, neuronal cells, renal tubular cells, etc. (FIGS. 9A-9F). Analysis of 29 immune cell types from normal donor peripheral blood mononuclear cells (PBMC) showed that CD94 transcript was predominantly present in NK cells, CD8+ terminal effector T cells, and gd T cells, and at lower levels in CD8+ effector T cells but was absent in dendritic cells, B cells, and monocytes (FIG. 9G).

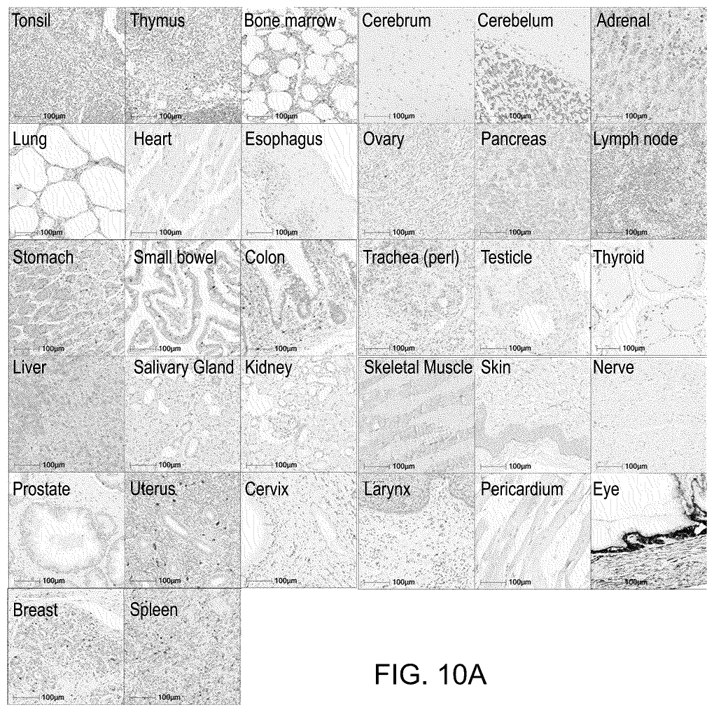

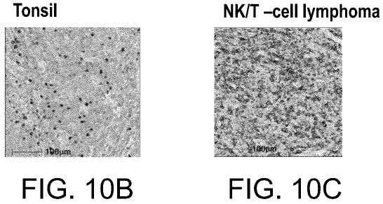

A tissue array containing 32 different organs derived from 3 different healthy donors was tested with commercial anti-CD94 monoclonal antibody (clone EPR21003, Abcam) at 1:400 titration. Standard immunohistochemistry protocol was followed and the signals were detected with Leica refine polymer detection kit. Positive signals (brown) were detected in scattered manner in reactive lymphocytes in different organs (lymph node, spleen, thymus, tonsil, breast, small bowel, colon, stomach, uterus, ovary, larynx, and esophagus). Cerebrum, cerebellum, adrenal, liver, thyroid, lung, pancreas, bone marrow, trachea, testicle, heart, skeletal muscle, skin, nerve, salivary gland, kidney, pericardium, eye, prostate, and cervix organs did not show positive signals (FIG. 10A). Tonsil derived from a normal donor (FIG. 10B) and NK/T-cell lymphoma tumor tissue derived from a patient (FIG. 10C) were used as positive controls and strong signals of CD94 were detected in both tissue slides.

CD94 protein expression was restricted in normal tissues. Consistent with scRNAseq data, immunohistochemical analysis of normal donor tissue array showed CD94 protein expression only in scattered in reactive lymphocytes in different organs (FIG. 10A). Strong staining for CD94 was detected in normal tonsil (FIG. 10B) and NK/T-cell lymphoma tissue (FIG. 10C) as expected.

### Example 6. Anti-CD94 Antibody Binding in NK-Cell Leukemia Cell Lines

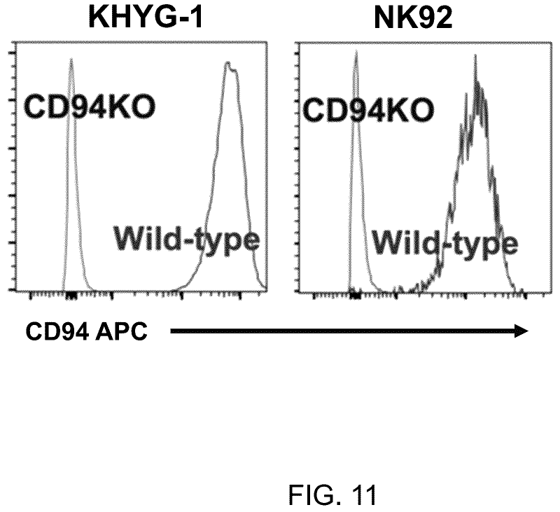

To assess the specificity of the CD94 monoclonal antibody, CRISPR-Cas9 technology was used to stably knock-out CD94 in two NK-cell leukemia cell lines (KHYG-1 and NK92). The binding specificity of anti-CD94 antibody was assessed by flow cytometry against wild-type KHYG-1 and NK92 cell lines and their isogenic cell lines with CD94 knock-out (KO) (FIG. 11). The anti-CD94 monoclonal antibody bound to the wild-type KHYG-1 and NK92 but not their isogeneic cell lines with CD94 knock-out confirming its specificity.

### Example 7. Anti-CD94 Targeting CARs Cytotoxic Activity In Vitro

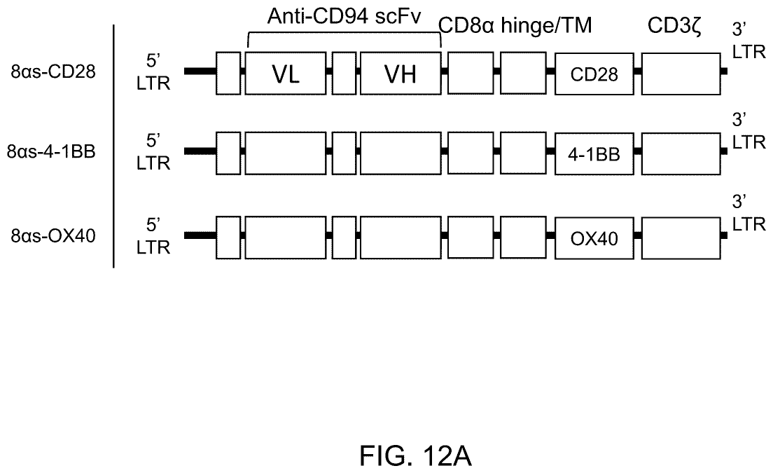

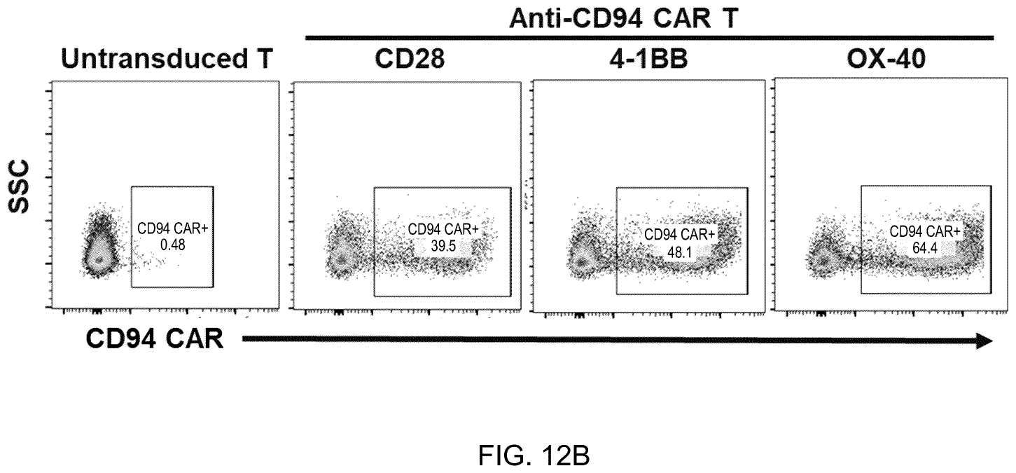

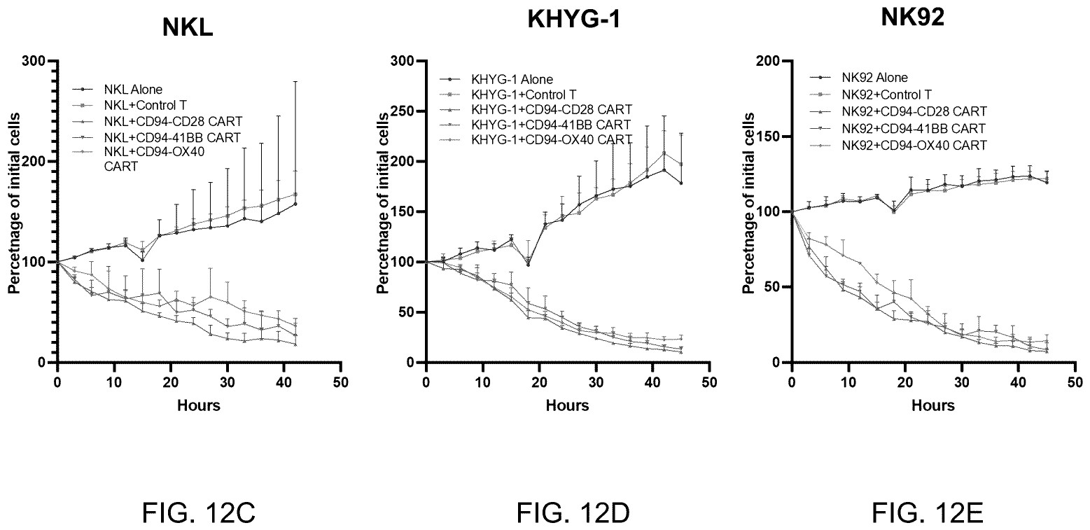

Three different CAR molecules were synthesized using the VH and VL from the anti-CD94 monoclonal antibody (FIG. 12A). Anti-CD94 scFv is linked to hinge/transmembrane domains of CD8α and costimulatory domains derived from CD28, 4-1BB, or OX-40. CD3z signaling domain is present in all three constructs. Anti-CD94 CAR molecules were lentivirally transduced into normal donor T cells and CAR transduction efficiency was assessed by flow cytometry after 72 hours after staining cells with recombinant human CD94 protein (FIG. 12B). Good transduction efficiency was noted with all three constructs. NK-cell leukemia cell lines (NKL-FIG. 12C, KHYG-1-FIG. 12D, and NK92-FIG. 12E) were stably transduced with red fluorescent protein (RFP). The tumor cells were then cultured in the presence or absence of CD94-targeting CAR T cells or untransduced T cells at an Effector:Target ratio of 0.5:1. Lysis or growth of tumor cells was monitored by serial imaging using the Incucyte Live-Cell Analysis System. Robust cytotoxic activity was observed against three different NK-cell leukemia cell lines (NKL, KHYG-1, and NK92) with anti-CD94 CAR T cells containing CD28, 4-1BB, or OX-40 costimulatory domains at a very low Effector:Target ratio of 0.5:1 (P<0.05) (FIGS. 12C-12E). In contrast, no significant lysis was observed with untransduced T cells.

### Example 8. Anti-CD94 Targeting CARs Cytotoxic Activity In Vivo

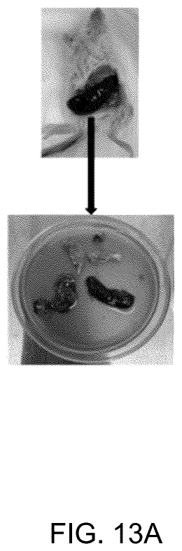

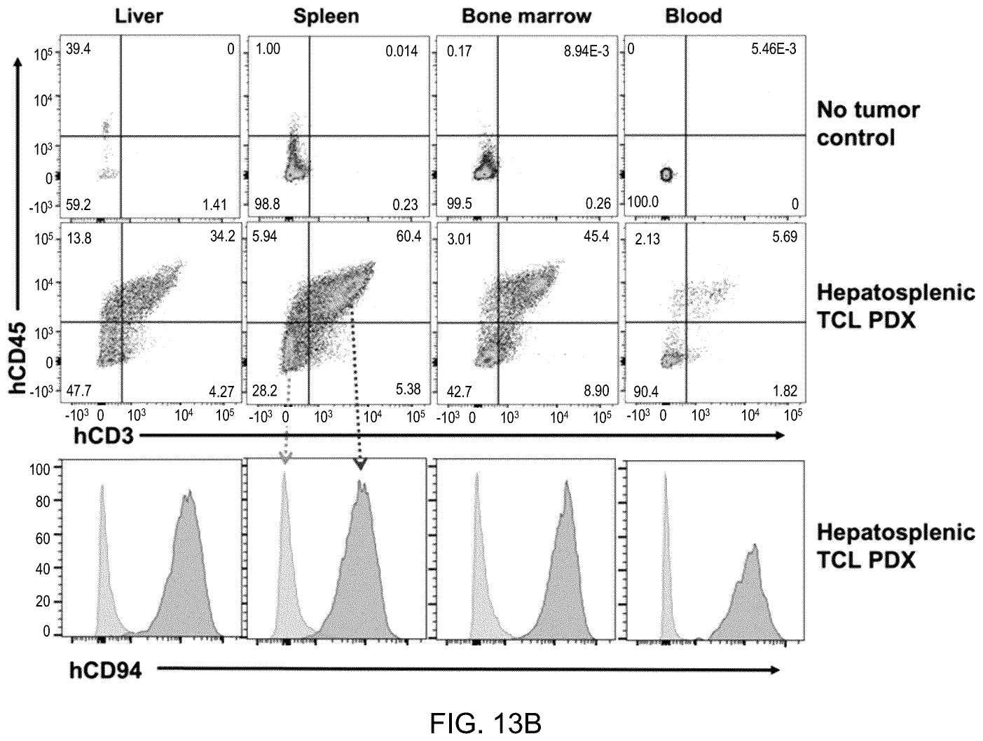

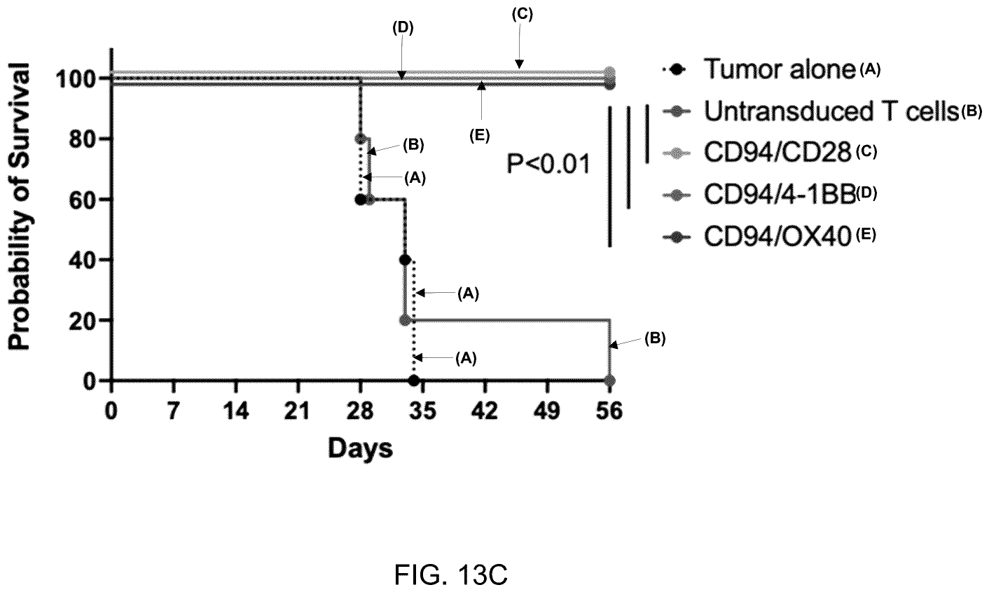

To assess the antitumor activity of CD94-targeting CAR T cells in vivo, a patient-derived xenograft model of hepatosplenic T cell lymphoma was established that was CD94 positive. Tumor cells (1×10⁶ tumor cells/mouse) from hepatosplenic T-cell lymphoma (TCL) PDX were injected intravenously via tail vein into NSG mice and mice were sacrificed on day 49 when they became moribund. Picture of liver and spleen at necropsy is shown in FIG. 13A. Intravenous injection of these tumor cells into NSG mice resulted in massive hepatosplenomegaly in mice and growth of tumor predominantly in liver, spleen, and bone marrow with minimal involvement of peripheral blood very similar to the clinical presentation in humans. These mice died of progressive tumor growth by about 50 days (data not shown). Single cell suspension from liver, spleen, bone marrow, and blood obtained at necropsy were analyzed by flow cytometry with human CD3 (hCD3), human CD45 (hCD45), and human CD94 (hCD94) antibodies (FIG. 13B). CD94 expression was compared between hCD3+hCD45+ cells vs. hCD3-hCD45-cells as shown. NSG mice that were not injected with tumor were used as controls. Tumor cells (1.5×10⁶ tumor cells/mouse) from hepatosplenic T-cell lymphoma (TCL) PDX were injected intravenously via tail vein into NSG mice (5 mice/group) and two weeks later, 10×10⁶ anti-CD94-targeting CAR+ T cells or untransduced T cells were administrated by intravenous injection. Antitumor effects of CAR T cells was assessed by monitoring the mice for survival. Log-rank test was used to evaluate the difference in survival between treatment groups. Treatment of NSG mice with CD94-targeting CAR T cells resulted in significant prolongation of survival (P<0.05) compared to mice that were untreated or mice treated with untransduced T cells. The survival at 56 days was 100% for mice treated with each of the three different CAR constructs with CD28, 4-1BB, or OX-40 costimulatory domains.

The practice of the present disclosure will employ, unless otherwise indicated, conventional techniques of cell biology, cell culture, molecular biology, transgenic biology, microbiology, recombinant DNA, and immunology, which are within the skill of the art. Such techniques are explained fully in the literature.

All of the references cited above, as well as all references cited herein, are incorporated herein by reference in their entireties.

Any examples provided herein are offered by way of illustration and not by way of limitation.

What is claimed is:

1. An antibody or antigen-binding fragment thereof capable of binding to CD94, wherein the antibody or antigen-binding fragment thereof comprises a complementary determining region (CDR) H1 comprising the amino acid sequence set forth in SEQ ID NO: 3 or SEQ ID NO: 19, a CDR H2 comprising the amino acid sequence set forth in SEQ ID NO: 4 or SEQ ID NO: 20, a CDR H3 comprising the amino acid sequence set forth in SEQ ID NO: 5 or SEQ ID NO: 21, a CDR L1 comprising the amino acid sequence set forth in SEQ ID NO: 6 or SEQ ID NO: 22, a CDR L2 comprising the amino acid sequence YTS or the amino acid sequence set forth in SEQ ID NO: 23 or SEQ ID NO: 24, and a CDR L3 comprising the amino acid sequence set forth in SEQ ID NO: 8.

2. The antibody or antigen-binding fragment thereof of claim 1, comprising a heavy chain variable region (VH) and a light chain variable region (VL), wherein the VH comprises an amino acid sequence having at least 85%, at least 90%, at least 91% at least 92%, at least 93%, at least 94%, at least 95%, at least 96%, at least 97%, at least 98%, at least 99%, or 100% sequence identity to SEQ ID NO: 1 and the VL comprises an amino acid sequence having at least 85%, at least 90%, at least 91%, at least 92%, at least 93%, at least 94%, at least 95%, at least 96%, at least 97%, at least 98%, at least 99%, or 100% sequence identity to SEQ ID NO: 2.

3. An antibody or antigen-binding fragment thereof capable of binding to CD94, wherein the antibody or antigen-binding fragment thereof comprises:
i) a CDR H1, CDR H2, and CDR H3 comprising the CDR H1, CDR H2, and CDR H3 amino acid sequences of SEQ ID NO: 1; and
ii) a CDR L1, CDR L2, and CDR L3 comprising the CDR L1, CDR L2, and CDR L3 amino acid sequences of SEQ ID NO: 2.

4. The antibody or antigen-binding fragment thereof of claim 3, wherein the CDRs are the Kabat-defined CDRs, the Chothia-defined CDRs, the AbM-defined CDRs, or the IMGT-defined CDRs.

5. The antibody or antigen-binding fragment of any of the previous claims, wherein the antibody or antigen-binding fragment comprises a VH comprising an amino acid sequence at least 85% identical to the amino acid sequence SEQ ID NO: 1.

6. The antibody or antigen-binding fragment of any of the previous claims, wherein the antibody or antigen-binding fragment comprises a VH comprising an amino acid sequence at least 90% identical to the amino acid sequence SEQ ID NO: 1.

7. The antibody or antigen-binding fragment thereof of any of the previous claims, wherein the antibody or antigen-binding fragment comprises a VH comprising an amino acid sequence at least 95% identical to the amino acid sequence SEQ ID NO: 1.

8. The antibody or antigen-binding fragment thereof of any of the previous claims, wherein the antibody or antigen-binding fragment comprises a VH comprising an amino acid sequence at least 98% identical to the amino acid sequence SEQ ID NO: 1.

9. The antibody or antigen-binding fragment thereof of any of the previous claims, wherein the antibody or antigen-binding fragment comprises a VH comprising an amino acid sequence at least 99% identical to the amino acid sequence SEQ ID NO: 1.

10. The antibody or antigen-binding fragment thereof of any of the previous claims, wherein the antibody or antigen-binding fragment comprises a VL comprising an amino acid sequence at least 85% identical to the amino acid sequence of SEQ ID NO: 2.

11. The antibody or antigen-binding fragment thereof of any of the previous claims, wherein the antibody or antigen-binding fragment comprises a VL comprising an amino acid sequence at least 90% identical to the amino acid sequence of SEQ ID NO: 2.

12. The antibody or antigen-binding fragment thereof of any of the previous claims, wherein the antibody or antigen-binding fragment comprises a VL comprising an amino acid sequence at least 95% identical to the amino acid sequence of SEQ ID NO: 2.

13. The antibody or antigen-binding fragment thereof of any of the previous claims, wherein the antibody or antigen-binding fragment comprises a VL comprising an amino acid sequence at least 98% identical to the amino acid sequence of SEQ ID NO: 2.

14. The antibody or antigen-binding fragment thereof of any of the previous claims, wherein the antibody or antigen-binding fragment comprises a VL comprising an amino acid sequence at least 99% identical to the amino acid sequence of SEQ ID NO: 2.

15. The antibody or antigen-binding fragment thereof of any one of claims 1-14, wherein the antibody or antigen-binding fragment thereof is human, humanized, or chimeric.

16. The antibody or antigen-binding fragment thereof of any one of claims 1-15, wherein the antibody or antigen-binding fragment thereof is an IgG antibody.

17. The antibody or antigen-binding fragment thereof of claim 16, wherein the IgG antibody is an IgG1 antibody or an IgG4 antibody.

18. The antibody or antigen-binding fragment thereof of any one of claims 1-15, wherein said antibody is an antigen-binding fragment of an antibody.

19. The antigen-binding fragment of an antibody of claim 18, wherein said fragment is selected from the group consisting of Fab, F(ab′)2, Fv, scFv, scFv-Fc, dsFv and a single domain molecule.

20. The antigen-binding fragment of an antibody of claim 19, wherein said fragment is a scFv.

21. The antigen-binding fragment of an antibody of claim 19, wherein said fragment is a Fab.

22. The antigen-binding fragment of an antibody of claim 18, wherein said fragment is an intrabody.

23. The antibody or antigen-binding fragment thereof of any one of claim 1-15 or 18-22, wherein said antigen-binding fragment is devoid of an Fc region.

24. The antibody or antigen-binding fragment thereof of any one of claim 1-15 or 18-23, comprising a VH and a VL on the same polypeptide chain.

25. The antibody or antigen-binding fragment thereof of claim 24, wherein the VH and VL are connected by a linker.

26. The antibody or antigen-binding fragment thereof of any one of claims 1-25, wherein the antibody or antigen-binding fragment thereof is conjugated to an agent selected from the group consisting of a therapeutic agent, a prodrug, a peptide, a protein, an enzyme, a virus, a lipid, a biological response modifier, a pharmaceutical agent, and PEG.

27. The antibody or antigen-binding fragment thereof of any one of claims 1-26, wherein the antibody or antigen-binding fragment thereof is a bispecific antibody.

28. A chimeric antigen receptor which comprises, from N-terminus to C-terminus: (a) an extracellular ligand-binding domain comprising an antigen-binding domain capable of binding to CD94; (b) a hinge; (c) a transmembrane domain; and (d) a cytoplasmic domain comprising a costimulatory domain and a signaling domain, wherein the antigen-binding domain comprises a CDR H1 comprising the amino acid sequence set forth in SEQ ID NO: 3 or SEQ ID NO: 19, a CDR H2 comprising the amino acid sequence set forth in SEQ ID NO: 4 or SEQ ID NO: 20, a CDR H3 comprising the amino acid sequence set forth in SEQ ID NO: 5 or SEQ ID NO: 21, a CDR L1 comprising the amino acid sequence set forth in SEQ ID NO: 6 or SEQ ID NO: 22, a CDR L2 comprising the amino acid sequence YTS, SEQ ID NO: 23, or SEQ ID NO: 24, and a CDR L3 comprising the amino acid sequence set forth in SEQ ID NO: 8.

29. The chimeric antigen receptor of claim 28, comprising a heavy chain variable region (VH) comprising the amino acid sequence of SEQ ID NO: 1 and a light chain variable region (VL) comprising the amino acid sequence of SEQ ID NO: 2.

30. The chimeric antigen receptor of any one of claim 28 or 29, wherein the antigen-binding domain is a scFv.

31. The chimeric antigen receptor of any one of claims 28-30, wherein the costimulatory domain is selected from the group consisting of a 4-1BB costimulatory domain, a CD28 costimulatory domain, or an OX40 costimulatory domain.

32. The chimeric antigen receptor of any one of claims 28-31, wherein the hinge, the transmembrane domain, or both, are from a CD8α polypeptide.

33. The chimeric antigen receptor of any one of claims 28-32, wherein the signaling domain comprises a CD3zeta signaling domain.

34. An engineered human T cell comprising a chimeric antigen receptor comprising, from N-terminus to C-terminus: (a) an extracellular ligand-binding domain comprising a scFv domain capable of binding to CD94, wherein the scFv domain comprises a VL and a VH;
(b) a hinge; (c) a transmembrane domain; and (d) a cytoplasmic domain comprising a costimulatory domain and a signaling domain, wherein the VH comprises a CDR H1 comprising the amino acid sequence set forth in SEQ ID NO: 3 or SEQ ID NO: 19, a CDR H2 comprising the amino acid sequence set forth in SEQ ID NO: 4 or SEQ ID NO: 20, a CDR H3 comprising the amino acid sequence set forth in SEQ ID NO: 5 or SEQ ID NO: 21, and wherein the VL comprises a CDR L1 comprising the amino acid sequence set forth in SEQ ID NO: 6 or SEQ ID NO: 22, a CDR L2 comprising the amino acid sequence YTS or the amino acid sequence set forth in SEQ ID NO: 23 or SEQ ID NO: 24, and a CDR L3 comprising the amino acid sequence set forth in SEQ ID NO: 8.

35. The engineered human T cell of claim 34, comprising a chimeric antigen receptor comprising an amino acid sequence having at least 85%, at least 90%, at least 91% at least 92%, at least 93%, at least 94%, at least 95%, at least 96%, at least 97%, at least 98%, at least 99%, or 100% sequence identity to SEQ ID NOs: 9, 56, or 57.

36. An isolated polynucleotide comprising a nucleic acid molecule encoding the VH or heavy chain of the antibody or antigen-binding fragment thereof of any one of claims 1-26.

37. The isolated polynucleotide of claim 36 further comprising a nucleic acid molecule encoding the VL or light chain of the antibody or antigen-binding fragment thereof of any one of claims 1-26.

38. An isolated polynucleotide comprising a nucleic acid molecule encoding the VL or light chain of the antibody or antigen-binding fragment thereof of any one of claims 1-26.

39. An isolated polynucleotide comprising a nucleic acid molecule encoding the chimeric antigen receptor of any one of claims 28-33.

40. An isolated vector comprising the polynucleotide of any one of claims 36-39.

41. A host cell comprising the polynucleotide of any one of claims 33-39, the vector of claim 40.

42. The host cell of claim 41, which is selected form the group consisting of CHO, HEK-293T, HeLa and BHK cells, optionally wherein the CHO cell is a CHO-K1SP cell.

43. A method of producing an antibody or antigen-binding fragment thereof capable of binding to CD94, the method comprising:
(a) culturing the cell of claim 41 or 42 in a cell culture under conditions which allow expression of the antibody or antigen-binding fragment thereof; and
(b) recovering the antibody or antigen-binding fragment thereof from said cell culture.

44. An antibody or antigen-binding fragment thereof obtainable by the method of claim 43.

45. A pharmaceutical composition comprising the antibody or antigen-binding fragment thereof of any one of claim 1-26 or 44, the chimeric antigen receptor of any one of claims 28-33, the engineered T cell of any one of claims 34-35, or the vector of claim 40 and a pharmaceutically acceptable excipient.

46. A method of treating cancer in an individual, the method comprising administering to the individual a therapeutically effective amount of the antibody or antigen-binding fragment thereof of any one of claim 1-26 or 44, the chimeric antigen receptor of any one of claims 28-33, the engineered T cell of any one of claims 34-35, or the pharmaceutical composition of claim 45.

47. The method of claim 46, wherein the cancer is a leukemia.

48. The method of claim 47, wherein the leukemia is T cell leukemia, T cell large granular leukemia, Natural Killer cell large granular leukemia, or Natural Killer cell leukemia.

49. The method of claim 46, wherein the cancer is a lymphoma.

50. The method of claim 49, wherein the lymphoma is T cell lymphoma, extranodal Natural Killer/T cell lymphoma, hepatosplenic T cell lymphoma, angioimmunoblastic T cell lymphoma, or anaplastic large cell lymphoma.

51. The method of claim 46, wherein the cancer is a CD94 expressing cancer.

52. A method of treating or preventing graft-rejection of a transplant in a patient, the method comprising administering to the individual therapeutically effective amount of the antibody or antigen-binding fragment thereof of any one of claim 1-26 or 44, the chimeric antigen receptor of any one of claims 28-33, the engineered T cell of any one of claims 34-35, or the pharmaceutical composition of claim 45.

53. The method of claim 52, wherein the transplant is an allogenic transplant.

54. The method of claim 52, wherein the transplant is an organ transplant.

55. The method of claim 52, wherein the transplant is a hematopoietic cell transplant.

56. The method of claim 52, wherein the transplant is an induced pluripotent cell therapy.

57. A method of modulating an immune response in a subject, the method comprising administering to the individual therapeutically effective amount of the antibody or antigen-binding fragment thereof of any one of claim 1-26 or 44, the chimeric antigen receptor of any one of claims 28-33, the engineered T cell of any one of claims 34-35, or the pharmaceutical composition of claim 45.

58. The method of claim 57, wherein the immune response is enhanced.

59. The method of claim 57, wherein the immune response is mediated by Natural Killer cells and/or T cells.

60. The method of claim 59, wherein the Natural Killer cells and/or T cells mediate the immune response of an autoimmune disease.

61. The method of claim 60, wherein the autoimmune disease is a Systemic Autoimmune Disease.

62. The method of claim 61, wherein the systemic autoimmune disease is systemic lupus erythematosus (SLE), Sjögren's Syndrome, Systemic Sclerosis, Rheumatoid Arthritis (RA), Multiple Sclerosis, type 1 diabetes mellitus (T1DM), or autoimmune liver disease (ALD).

63. A method of treating cancer in an individual, the method comprising administering to the individual a therapeutically effective amount of an immune cell engineered to express the chimeric antigen receptor of any one of claims 28-33.

64. The method of claim 63, wherein the immune cell is a Natural Killer cell, a Natural Killer T cell, a macrophage, or a monocyte.
